(19) United States
(12) Patent Application Publication
(10) Pub. No.: US 2016/0317341 A1
(43) Pub. Date: Nov. 3, 2016

(54) Multi-functional wearable-implemented method to provide fetus's life advancement and enhancements for pregnant mothers

(71) Applicant: Adan Galvan, Cupertino, CA (US)

(72) Inventor: Adan Galvan, Cupertino, CA (US)

(21) Appl. No.: 14/702,742

(22) Filed: May 3, 2015

(51) Int. Cl. A61F 5/02

(52) U.S. Cl. CPC A61F 5/028; A61F 5/02

(57) ABSTRACT

The invention works using various items such as a scalable controller board, Ai processing chip, Ai software, firmware, programming language, web spider bots, pCell wireless chip, health sensors, lateral belt support and a casing to house the items creating an extension of the human mind and body interconnecting the human physiology with artificial intelligence and Neuro-synaptic and cognitive computing furthering our health, education and advancement of our civilization.

### FIELD OF THE INVENTION

The present invention is in the field of prenatal back supports.

### BACKGROUND OF THE INVENTION

The present invention is in the field of prenatal care. More particularly, the present invention is in the technical field of prenatal back support with audio to play to the unborn child.

### SUMMARY

The present invention is used to support women's back during three trimesters of pregnancy to prevent pregnancy complications and back pain.

### BRIEF DESCRIPTION OF DRAWINGS

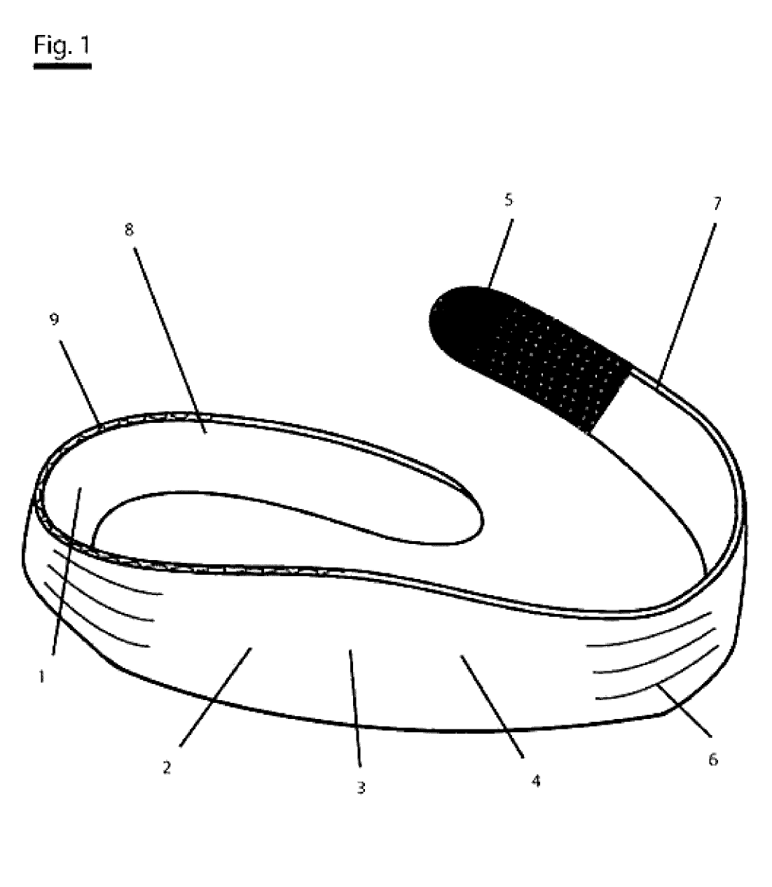

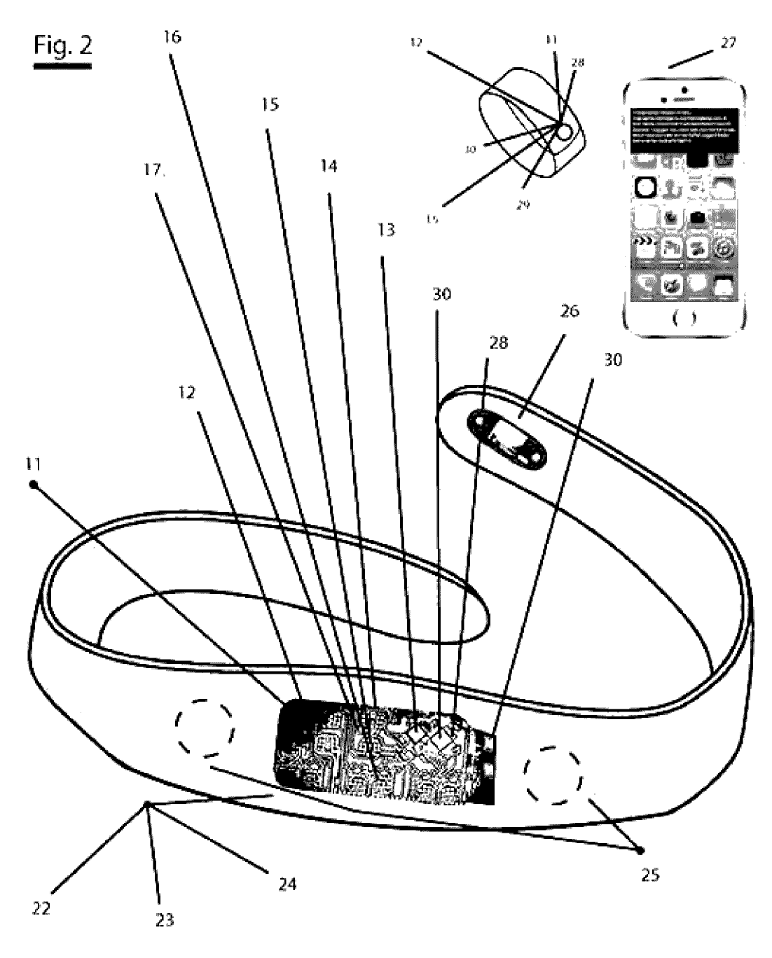

The relation between the invention FIG. 1 and FIG. 2 and the Cloth item 1 is that the Cloth item 1 holds part of the invention together.

The relation between the invention FIG. 1 and FIG. 2 and e-Textiles item 2 is that e-Textiles item 2 use micro-controlled sensors and actuators which transmit data to and from each component within the ecosystem, and when necessary outside of the ecosystem.

The relation between the Copper Oxide item 3 and the cloth item 1 is that the cloth item 1 uses the properties of copper through an infusion process, which then becomes part of the fibers in the cloth item 1, killing 99.9% of germs throughout the invention FIG. 1 and FIG. 2.

The relation between the Shape-Memory and Polymers item 4 and the invention FIG. 1 and FIG. 2 is that cloth item 1 which is used on the invention FIG. 1 and FIG. 2, creates a softer, more flexible material and shapes to the contours of the wearer when the invention FIG. 1 and FIG. 2, is worn.

The relation between the Velcro item 5 and the invention FIG. 1 and FIG. 2, is that it provides a secure locking mechanism when the invention FIG. 1 and FIG. 2 has been adjusted for comfort by the wearer.

The relation between the Elastic Material item 6 and the invention FIG. 1 and FIG. 2, is that the Elastic Material item 6 allows for the invention FIG. 1 and FIG. 2 fitting many shapes, sizes and figures of the wearer.

The relation between the Peruvian Pima Cotton Liner item 7, and the invention FIG. 1 and FIG. 2, is that it provides a luxurious look and feel while having the highest quality to breathe properly so that it stays cool in the summer and warms in the winter.

The relation between the Threat 9 and the invention FIG. 1 and FIG. 2, is that it holds the entire invention together while all components are working harmoniously in the ecosystem.

The relation between the Microcontroller with bios and firmware item 11 and the invention FIG. 1 and FIG. 2, is that it provides a scalable platform, in which unlimited technologies can work in the ecosystem of the invention FIG. 1 and FIG. 2 and broaden the capabilities of the invention FIG. 1 and FIG. 2. In further detail, the bios and firmware is essentially a ROM chip. Where software instructions can reside in non-volatile memory which will make all hardware and software interact both harmoniously as well as symbiotically.

The relation between the Qualcomm Neuromorphic Chip item 12 and the invention FIG. 1 and FIG. 2, is that it assists the companion mode within the ecosystem of the invention FIG. 1 and FIG. 2, in which the wearer will be given real time suggestions on ways to improve the wearers health through heuristics and direction.

The relation between the Memory Chip item 13 and the Microcontroller with bios and firmware item 11 and the invention FIG. 1 and FIG. 2, is that is stores users data locally to add an extra layer of security as the wearers personal information is stored within the invention FIG. 1 and FIG. 2 for user experience improvements.

The relation between the Class II Bluetooth Chip item 14 and the invention FIG. 1 and FIG. 2, is that it gives interconnectivity to the ecosystem so the invention can offer endless possibilities to the wearer that focuses on the health and well-being of the wearer.

The relation between the Microphone MIC, Audio Decoder, Audio Codec and Amplifier Chip item 15 and the Microcontroller with bios and firmware item 11 and the invention FIG. 1 and FIG. 2, is so the wearer can speak commands using Siri which is enabled on iPhone or S-Voice which is enabled on Android devices, while being able to use the invention FIG. 1 and FIG. 2 hands free and provide a richer experience to the wearer. As an example, the wearer can say, Siri, dinner recipe, and Siri will give options and when the wearer selects an option verbally, Siri will walk the wearer through the recipe that suppresses the wearers diseases such as acid reducing recipe to prevent the wearers hereditary disease GERD from progressing. The wearer can also speak type food journals, recorded voices to play to the fetus and other endless possibilities.

The relation between the Auto Length Accelerometer-Based Board item 16 and the invention FIG. 1 and FIG. 2, is that it gives the wearer a full scope of their progress real time, such as gauging total and interval distance; laps; calories burned; session average, interval and length pace; and stroke count, including number of strokes per length and interval and session averages.

The relation between the Altimeter item 17 and the invention FIG. 1 and FIG. 2, is that it gives the wearer peace of mind know they will never get lost as it can be used along with a topographic map, which can help to verify one's location. It is more reliable, and often more accurate, than a GPS receiver for measuring altitude. It's perfect for hiking or climbing.

The relation between the 6-Axis Accelerometer and the Gyroscope Boards item 19 and the invention FIG. 1 and FIG. 2, is that it will provide a motion interface as technology matures to deliver a richer and more interactive experience for the wearer. It is also designed for the low power, low cost, and high performance requirements of consumer electronics products. This means as newer technology emerges the invention FIG. 1 and FIG. 2 can organically grow with the demands of the consumer.

The relation between the Rechargeable Aluminum-Ion Battery with Integrated Power-Saving Accelerometer Boards and the invention FIG. 1 and FIG. 2, is so the wearer can use the invention FIG. 1 and FIG. 2 for weeks at a time without charging.

The relation between the Health Sensors item 21 and the invention FIG. 1 and FIG. 2 is that the wearer will be monitored at all times through the invention FIG. 1 and FIG. 2 in case health vitals get into severe readings, the invention FIG. 1 and FIG. 2 can then act and call 911 or contact a family or relative if those options are chosen. It can additional be used for the wearer to keep track of heart rates and respiratory information for improvement on health.

The relation between the Calorie Counter item 22 and the invention FIG. 1 and FIG. 2, is that the user can count calories to work towards losing weight and lower their risk of being obese. There are other methods for using the calorie counter as it would be up to the user on how to implement the tool.

The relation between the Bluetooth® Smart Technology 24 and the Microcontroller with bios and firmware item 11 and Class II Bluetooth Chip item 14 and the invention FIG. 1 and FIG. 2, is so that the wearer is able to keep up with technology and the invention FIG. 1 and FIG. 2 won't become obsolete. The technology is able to connect the wearer's life with technology.

The relation between the Sensor Patch item 26, the invention FIG. 1 and FIG. 2, the Class II Bluetooth Chip item 14 and the invention item 27, is so that the invention FIG. 1 and FIG. 2 can read and transmit blood chemistry data to the wearers Smartphone through the invention item 27. This is revolutionary as the invention FIG. 1 and FIG. 2 can continuously monitor metabolic panel data and transmit it to providers.

The relation between the detachable Bluetooth Speakers item 25 and the invention item 27 and the invention FIG. 1 and FIG. 2, is so that the wearer can play sound to her fetus and access an unlimited amount of classical music, educational audio, greetings in foreign languages for culture tolerance and other very important purposes.

The relation between the invention FIG. 1 and FIG. 2, Microcontroller with bios and firmware item 11, GPS chip item 28, Small GPS wrist band item 29 and the Smartphone App 27, is so that parents can keep track of their children at all times.

The relation between the invention FIG. 1 and FIG. 2, Microcontroller with bios and firmware item 11, GPS chip item 28, pCell chip and board and item 27 is so that the wearer will be connected through the World Wide Web interconnecting the two.

The relation between the invention FIG. 1 and FIG. 2 and customizable utility compartments item 31, is so item 31 can attach and detach from FIG. 1. and FIG. 2 using item 5 as part of the invention to offer a broader scope of applications in different industries.

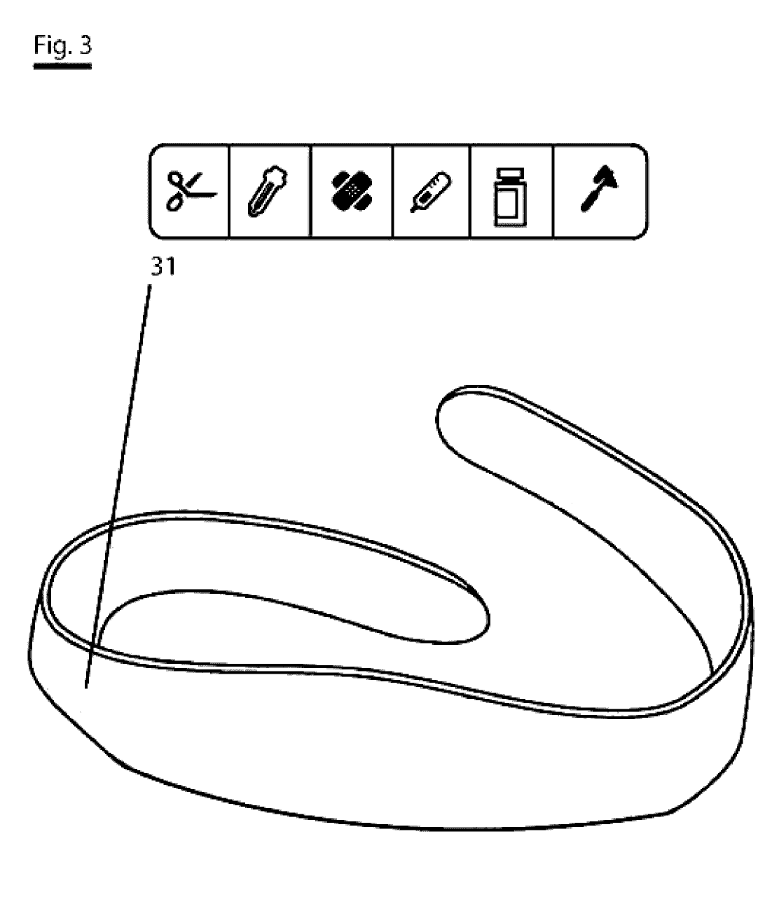

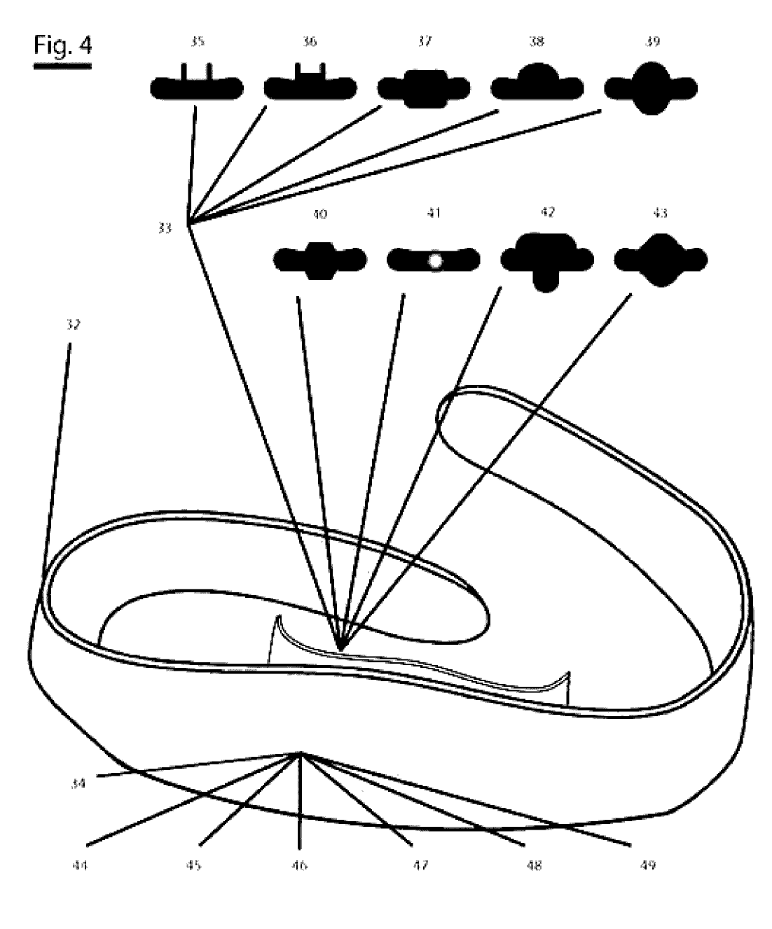

The relation between the invention FIG. 1, FIG. 2, FIG. 3, FIG. 4, stationary layer item 32, detachable layer item 33, various material options item 34, is so that the invention can be adaptable to each wearer who prefers different angled material or shaped form of the invention as seen in FIG. 4.

The relation between the invention FIG. 1 FIG. 2, FIG. 3, FIG. 4, is so that the stationary layer item 32, detachable layer item 33, various material options item 34 provides a more custom fit and comfortable experience.

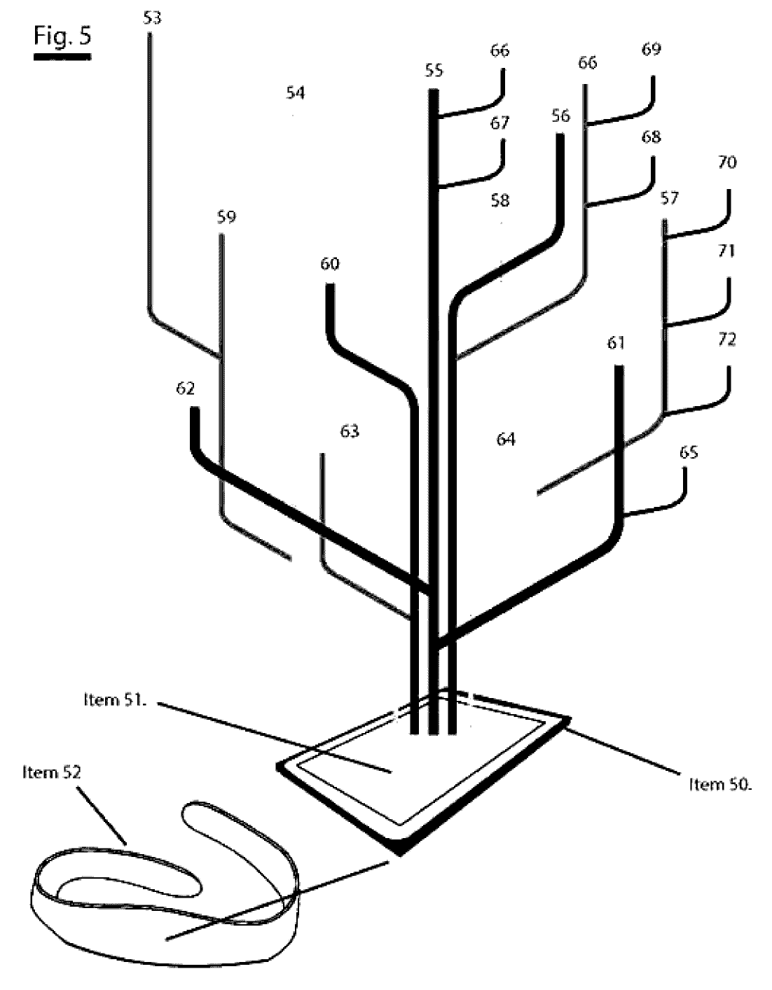

The relation between the invention FIG. 1, FIG. 2 and FIG. 5, is so that the invention can communicate through pCell Chip and Board item 30 with any internet connecting device to inner connect the wearer and provide pertinent data to educate and encourage a healthy lifestyle.

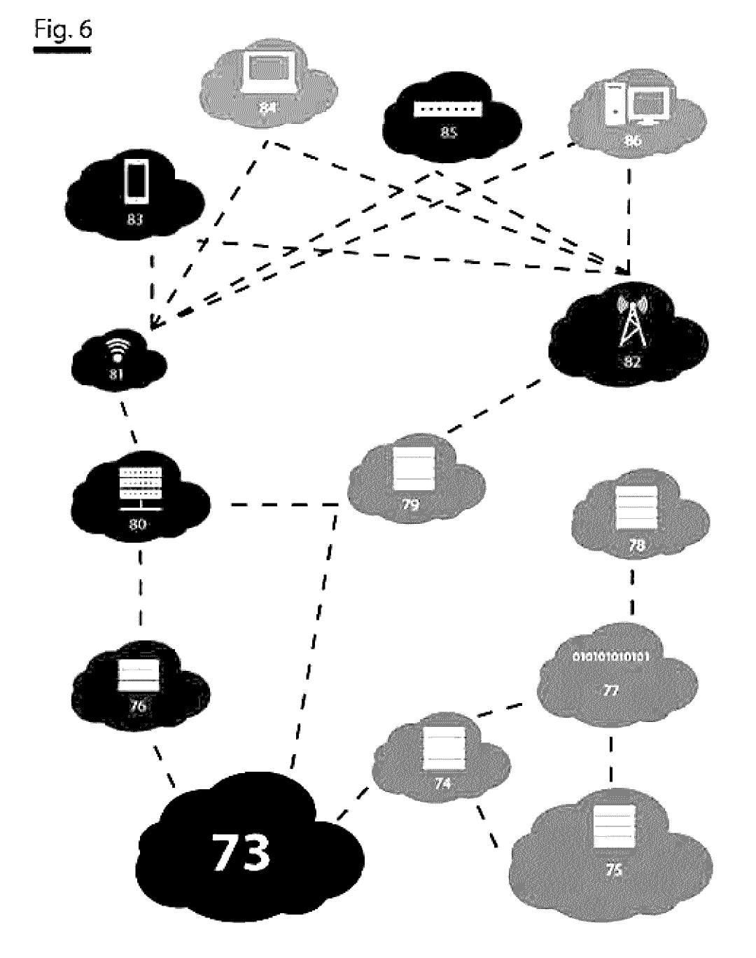

The relation between the invention FIG. 1, FIG. 2, FIG. 5 and FIG. 6 is that the cloud network provides an interconnect of information to the prenatal support belt and the Smartphone app FIG. 2 item 27.

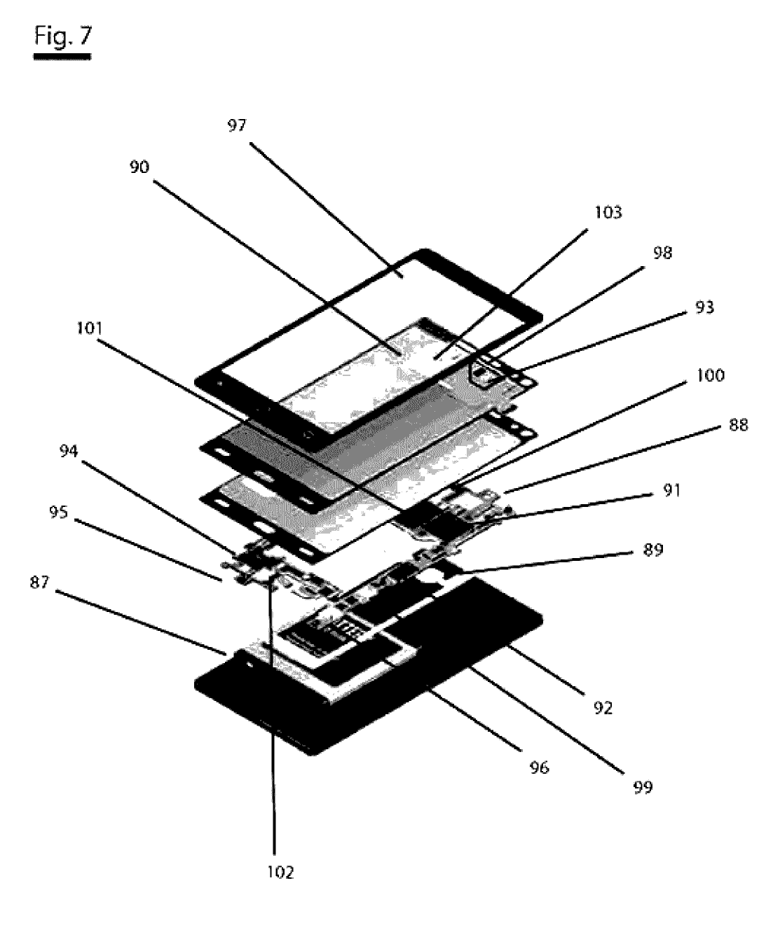

The relation between the invention FIG. 1, FIG. 2, FIG. 5, FIG. 6 and FIG. 7 gives the wearer the ability to use their Smartphone FIG. 7 to connect through their cellular carrier item 65, Item 52—WWW, Item 54—Cloud, Item 59—Mobile, Item 60—Notebook, Item 61—Invention, Item 62—PC, Item 63—ISP, Item 64—Router or Modem, FIG. 2 to provide access to a world of information about health and other features using a structured Smartphone app FIG. 2 item 27.

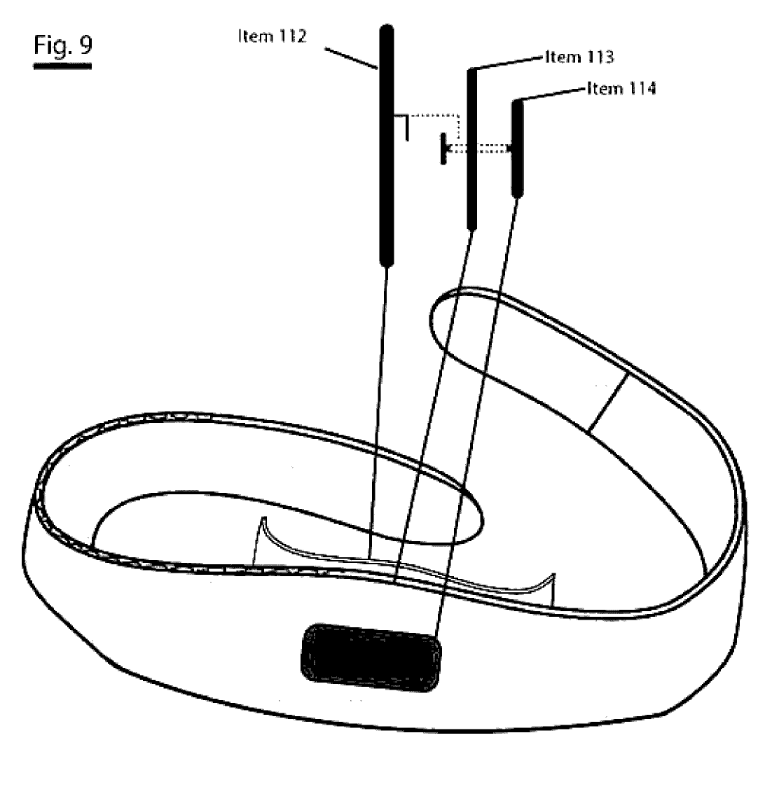

The relation between FIG. 9, it gives the wearer the option to detach back, which can consist of various chiropractic preferred material and therapeutic back relief options from item 91 and item 92. Where item 92 has a casing that has a male rod that slips through item 91 and connects item 90 connecting all items, item 90, item 91 and item 92 to form part of the invention.

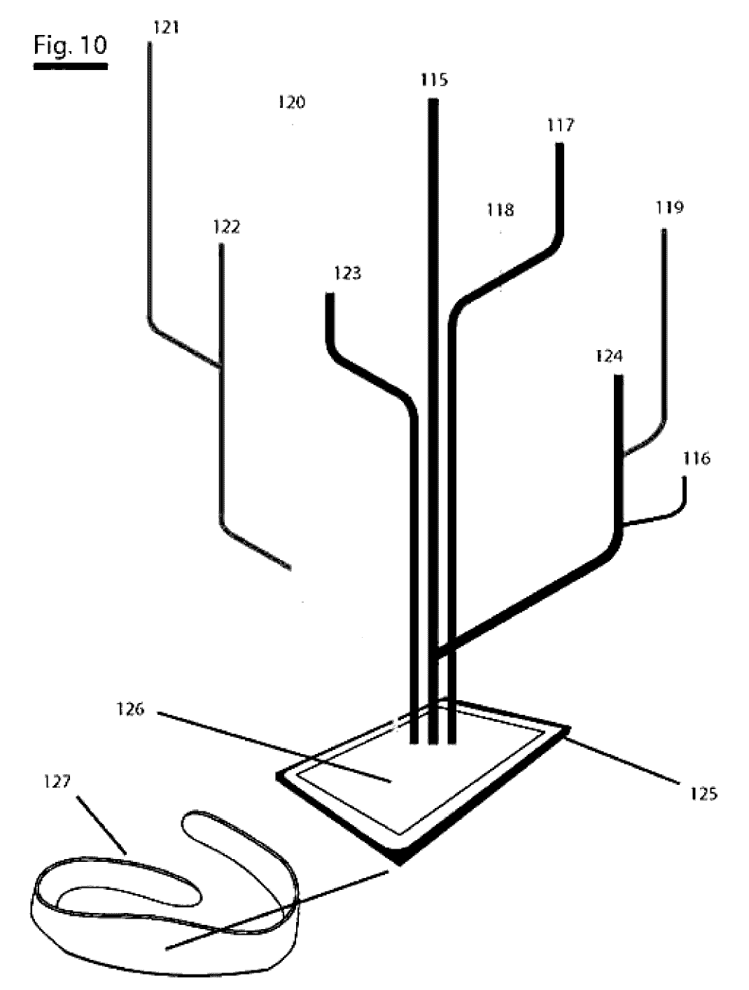

The relation between FIG. 10, is that they work in conjunction with other assembled boards to offer more medical features as part of the invention. The relation between item 15-24 is that they're all assembled inside the item 125, making item 127 which can then be embedded into item 125 to form part of the invention.

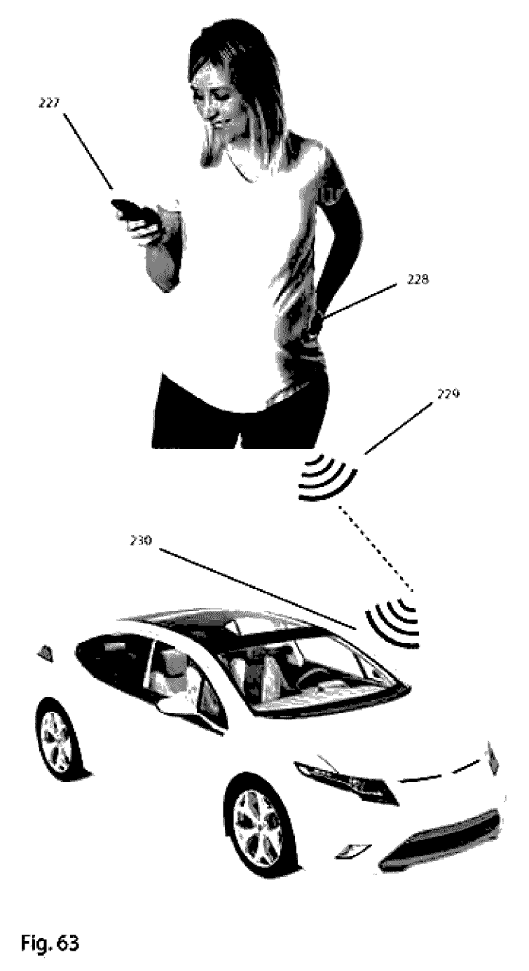

The relation between FIG. 63-FIG. 71 the invention and the various items can connect to any Bluetooth/Cellular enabled device to give the wearer more control over various items.

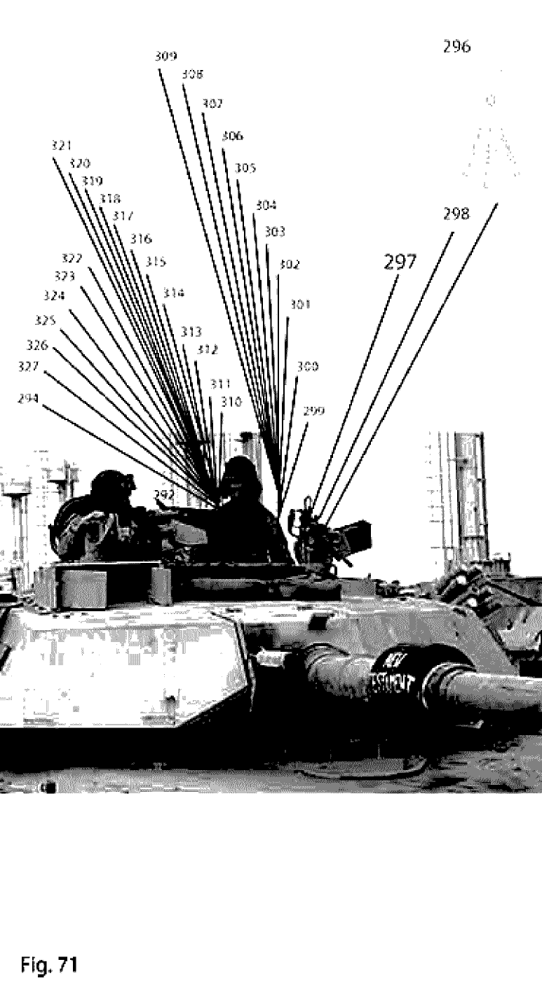

The relation between all parts, functions, technology and programming brings to life the invention FIG. 1-FIG. 71 and all items 1-327.

### DETAILED DESCRIPTION OF EMBODIMENTS

The invention gives life to an ecosystem where software, hardware technologies, information, programming language and human interaction work harmoniously to provide physiological heuristic solutions. Furthermore, as a result of the ecosystem, the invention can easily be scaled and provide new functions as health information and research develops, human interaction and understand and technology in both programming and hardware.

In one example of the invention, it incorporates e-textile technology. This allows the invention to monitor health vitals such as heart rate, respiration, skin temperature and even measure posture using micro-controlled sensors and actuators. Being in tune with our bodies is one aspect of the invention that helps the wearer be more cognizant and health conscious.

In another example of the invention, the material incorporates Peruvian pima cotton. Part of the invention is designed to provide comfort for the wearer. Peruvian pima cotton is the world's finest Pima, prized for its exceptional softness and brilliant luster, as well as its durability. It also has the benefit of being able to breathe. This allows it to stay cool in the summer and warm in the winter.

In another example of the invention, health sensors are embedded into the material. These health sensors work in conjunction with other miniature circuits, microcontroller functions, front-end amplification and wireless data transmission technologies. This gives the wearer a great advantage over their health to control it and empowers the wearer to achieve a range of health outcomes through heuristics health solutions.

In another example of the invention, embedded into it are small patch sensors. These sensors can read and transmit blood chemistry data to an external device like a Smartphone using Bluetooth technology. This is extremely vital as it can provide blood biomarkers such as glucose and potassium. Having real time data of blood levels can benefit a range of people from diabetics to athletes to those who just want to ensure their body is optimized appropriately. This helps the wearer continue to work towards a healthier body and healthier living.

In yet another example of the invention, the material that makes up my invention is infused with copper oxide. Through this process, copper oxide is combined with fibers of the material. There is then a chemical reaction and the copper elements solidify as part of the materials properties. The benefit of using copper in the invention is to create an anti-bacterial effect. This is beneficial as the invention kills 99.9% bacteria and other germs that can potentially harm the wearer and weaken the wearer's immune system during pregnancy.

In another example of the invention, the inner detachable support liner can be made of any material including SEE FIG. 4, item 34, but not limited to, clear gel composite, copper fibers, air pocket material, polyester, crepe, denim, satin, cotton, even micro-foam. This allows the invention to offer every possible combination of fabric to accommodate any preference.

In yet another example of the invention, the inner detachable support liner can be made into any shape or angle to accommodate any body shape, SEE FIG. 4, item 33. The shapes include, but not limited to, squares, octagons, pentagons, rectangles, triangles, circles, hexagons, heptagons, Isosceles Triangle, Scalene Triangle, Right Triangle, Obtuse Triangle, Acute Triangle, Rhombus, Parallelogram, Trapezoid, Irregular Triangle, Irregular Quadrilateral, Irregular Pentagon, Irregular Hexagon, Nonagon, Triangular Prism, Hexagonal Prism, Tetrahedron, Octahedron and the endless other shapes.

In yet another example of the invention, the inner detachable support liner can be made into unlimited grades of density, SEE FIG. 4, item 34, these densities include, but not limited to, gel, micro-foam, memory foam, latex, cellulose, synthetic and even micro-balls, back relief gel-beads and other composites and therapeutic material. The invention provides a broad scope of supporting enhancements, making it nearly impossible to duplicate without copying aspects of it in part or in whole.

In one example of the invention, an embedded microcontroller is used. The microcontroller is a mixed-signal controller. The microcontroller can be built around any CPU, to provide a design that's low cost and, specifically, low power consumption.

The microcontroller comes in a variety of configurations featuring the usual peripherals, internal oscillator, timers including PWM, watchdog, etc., and brownout reset circuitry. This allows for the invention to scale as new technologies emerge while providing an ecosystem in which all hardware, software, advanced technologies, programming language and human advancement can thrive.

In another example of the invention, integrated is a detachable smart compartment that can house the following, but not limited to:

Medical Kits, Breathing and Respiratory Supplies, Infection Control Supplies, Medical Diagnostic Equipment, Medical Needles and Syringes, Surgery Center & Urgent Care, Surgical Procedure Instruments, Surgical Procedure Supplies, Wound Care Healing Management, Wound Care Healing Supplies, Miniaturized Oxygen Kits, Inflatable flints, Airway Management Sets, Spinal Collars, Mini-mag lights, Mini-Defibrillators, Surgical gloves, Mini-scissors, Mini-fingertip reader, Blood sugar glucose test strips, Virological blood test strips, Bacteriological blood test strips, Serological blood test strips, Stool blood test strips, Urine test blood, test strips.

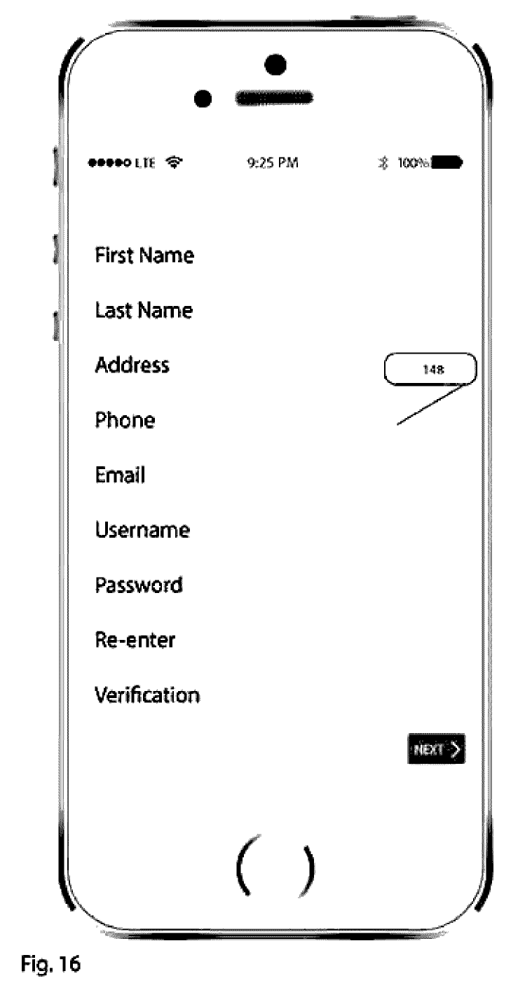

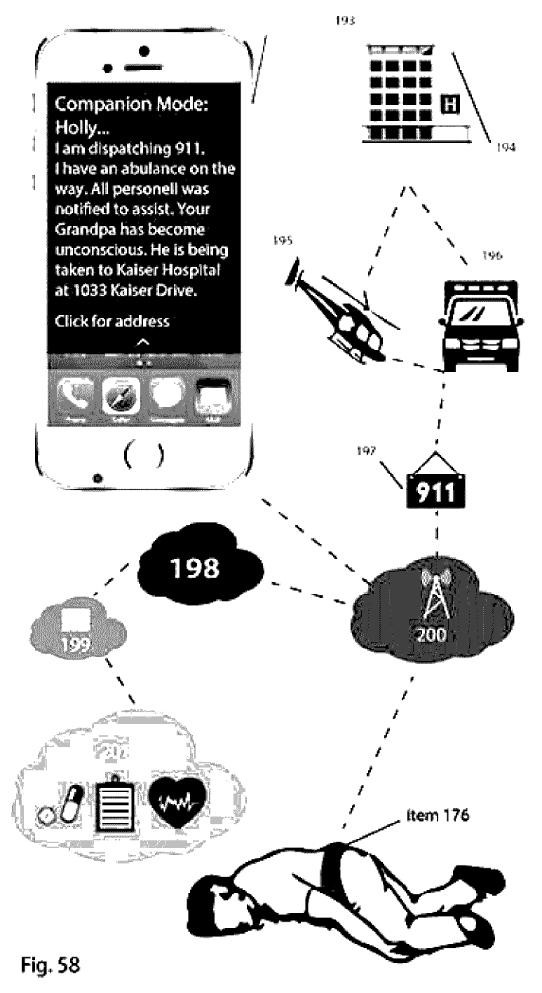

In another example of the invention, embedded into the microcontroller is a pCell board and chip. pCell technology is effectively mobile fiber. pCell signals can achieve 35× the LTE spectral efficiency and outperforms any existing 4G technology while still being backward compatible with 1×, 3G, 4G, 5G, GSM/EDGE; UMTS/HSPA+; DC-HSDPA; CDMA EV-DO Rev. A and Rev. B. This allows the invention to connect to any wireless carrier's cell tower FIG. 6 item 65, giving life to a wide spectrum of features and possibilities. As an example, if the invention FIG. 6 item 61 detects that the wearer's heart rate drops below a level that renders the person unconscious; this can be detected through the inventions FIG. 6 item 61 sensors FIG. 2 item 26. It will then automatically connect to the surrounding cell towers FIG. 6 item 65. It will then connect to the cloud item 54, accesses the health information the wearer inputted into the Smartphone app FIG. 2 item 27, i.e. diseases they have, blood readings, recent appointments, medications etc., the Smartphone app FIG. 2 item 27 will then transmit that vital information to the ambulance and hospital so the medical professionals will be prepared and proactively be ready to act immediately to give the wearer a better chance at recovering and/or surviving. It can also be programmed to send a message to the wearers spouse, relative etc. that is designated by the wearer at registration see FIG. 16, to receive the updates in case of an emergency, SEE FIG. 58. With the inventions wireless capability and functionality, the possibilities are endless.

In another example of the invention, integrated into the microcontroller is pure aluminum-ion battery technology FIG. 2 item 20. This allows the battery to last through all activities and has GPS Nano-Spider technology and accelerometer boards that have advanced power-saving features for ultra-low-power applications. These features include low-power mode, auto wake-up function and FIFO buffer that can be used to store data, thus reducing the host processor loading and system power consumption. These accelerometer boards are engineered to withstand stresses of modern mobile applications.

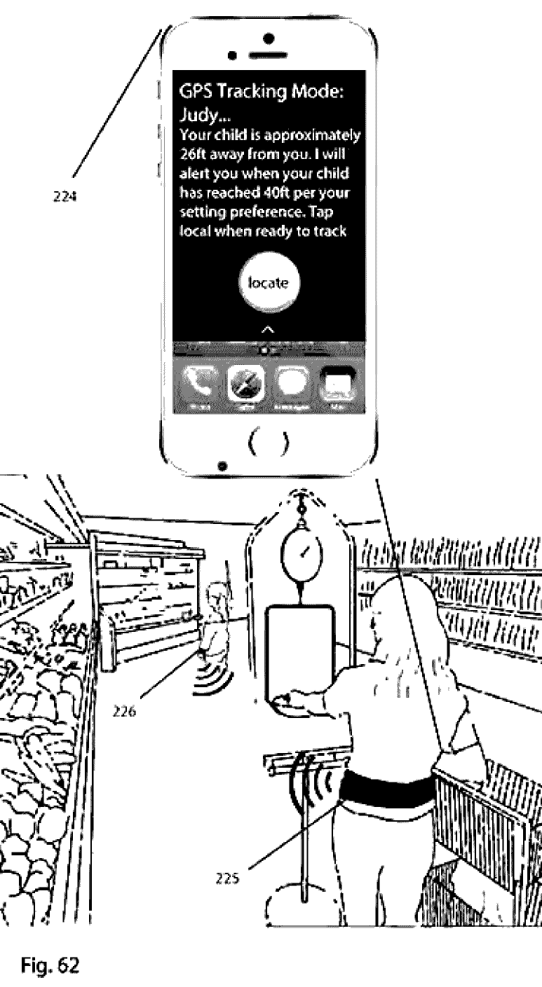

In yet another example of the invention, there are small GPS wrist band FIG. 2 item 29 that can fit around any wrist to track that person SEE FIG. 62.

In another example of the invention, integrated into the microcontroller is a 6-axis accelerometer board which includes a Gyroscope. It's designed for the low power, low cost, and high performance requirements of consumer electronics products.

The unit combines a 3-axis gyroscope and a 3-axis accelerometer board on the same silicon die together with an onboard Digital Motion Processor DMP capable of processing complex 9-axis Motion Fusion algorithms. The parts' integrated 9-axis Motion Fusion algorithms access external magnetometers allowing the devices to gather a full set of sensor data.

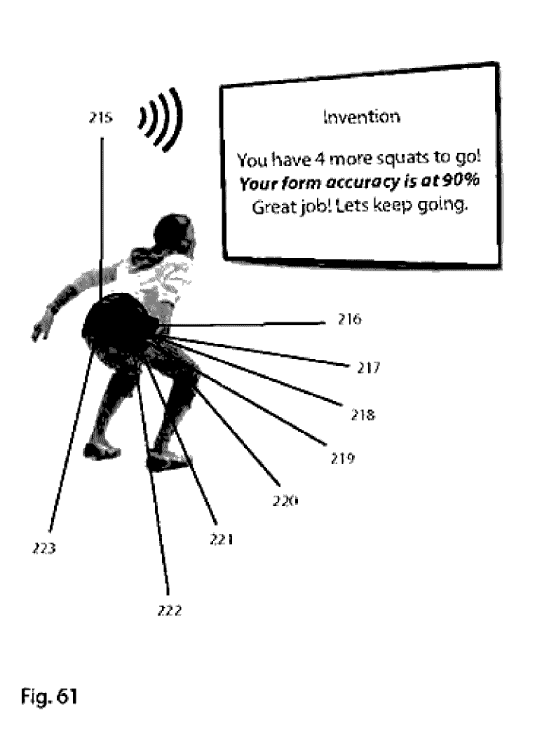

The combination of these boards offer various integration of new sensor technology. It allows the wearer to track their exercise routines, movement and other activities to have a better grasp of their efficiency during workouts. This ensures the best results for the wearer's health and body, SEE FIG. 61.

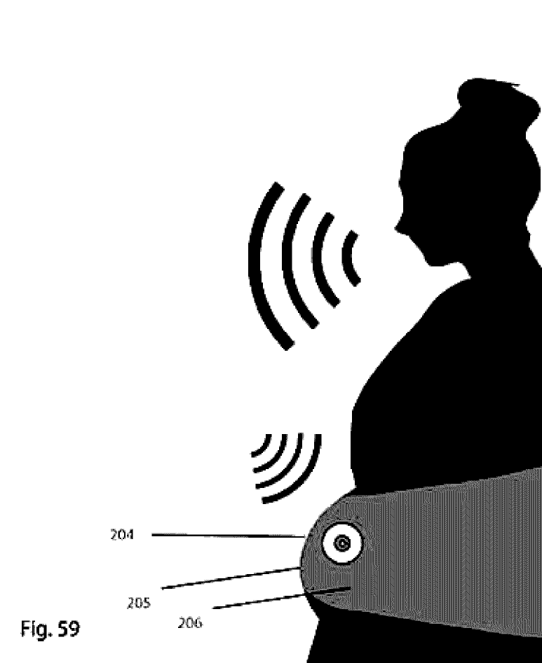

In another example of the invention, it integrates a microphone MIC, audio decoder, audio codec and amplifier chip which enable the wearer to give voice commands. When the microphone MIC, audio decoder, audio codec and amplifier chip works in conjunction with the microcontroller, it allows for the invention to be voice command capable through software engineering and syntax instruction using various programming languages. This gives the wearer the ability of using the invention hands-free on various electronics to display all data and interact using just the wearer's voice, SEE FIG. 59.

In yet another example of the invention, integrated into the microcontroller is an auto length accelerometer-based board. This technology automatically gauges total and interval distance; laps; calories burned; session average, interval and length pace; and stroke count, including number of strokes per length and interval and session averages.

In one example of the invention, integrated into the microcontroller is class II Bluetooth chip. This gives the ability to use wireless Bluetooth technology and connect to any Bluetooth enabled device, SEE FIG. 61.

In another example of the invention, integrated into the microcontroller is Bluetooth® Smart technology. This new technology enables exciting new possibilities for developers in a wide range of markets including sports and fitness, health, consumer electronics, and more. It is opening up appcessory development in a way that is endless in possibilities, SEE FIG. 61.

In another example of the invention, using a microcontroller, class II Bluetooth chip and program engineering allows for voice command with Siri, an application for Apple's iOS which works as a personal assistant and knowledge navigator. The application uses a natural language user interface to answer questions, make recommendations, and perform actions by delegating requests to a set of Web services. This gives the wearer a richer experience as well as giving the invention the ability to act as a companion, which research has shown to effectively encourage action making the invention patentable subject matter.

In another example of the invention, using a microcontroller, class II Bluetooth chip and program engineering allows for voice command with s-voice. S Voice allows you to improve the speed of your communications as well as increasing productivity with quick shortcuts to vital settings, all while leaving your hands free to deal with other tasks. This gives the wearer a richer experience as well as giving the invention the ability to act as a companion, which research has shown to effectively encourage action, SEE FIG. 61.

In yet another example of the invention, integrated is an altimeter. This is a technology that measures the altitude of an object above a fixed level. The measurement of altitude is called altimetry, which is related to the term bathymetry, the measurement of depth underwater. When this technology is used in the inventions ecosystem, the invention is able to continuously track all GPS satellites in view and provides real-time positioning data in the standard industry format defined by the U.S. National Marine Electronics Association NMEA. Some other features are as followed, but not limited to: Low noise amplifier LNA, surface acoustic wave SAW filter, temperature controlled crystal oscillator TCXO and real time clock RTC crystal, a power management unit, and radio frequency RF shielding. Superior sensitivity and performance—Offers accuracy of approximately one meter, achieves a rapid time to first fix TTFF of less than one second and tracking sensitivity of −163 dBm.

Continuous connectivity with minimal power consumption—It achieves a state of near continuous availability by detecting changes in context, temperature and satellite signals by maintaining and opportunistically updating its internal fine time, frequency, and satellite data, while consuming microwatts of battery power.

Improving marginal signal conditions—It includes a system to increase noise immunity even under marginal signal conditions, such as urban canyons, under dense foliage or a rapidly-changing receiver position.

In another example of the invention, there is a memory chip embedded into the microcontroller to save preferences, data and usage information. As an example, the data and preferences that are saved by the wearer can then automatically connect through Bluetooth to a Tesla electric car. The invention then accesses the wearer's preferences and automatically sets seat position, temperature, radio stations and other wearer saved preferences. This gives the wearer full control over technology and provides a richer experience, SEE FIG. 63.

In another example of the invention, there is a feature called, companion mode. When enabled, the invention uses a processes and the Smartphone app FIG. 2 item 27. While in companion mode, the chip uses a suggestive reasoning hierarchy map based on permissions, instructions, selected options and information about the wearer through Ai software, and will act as a companion.

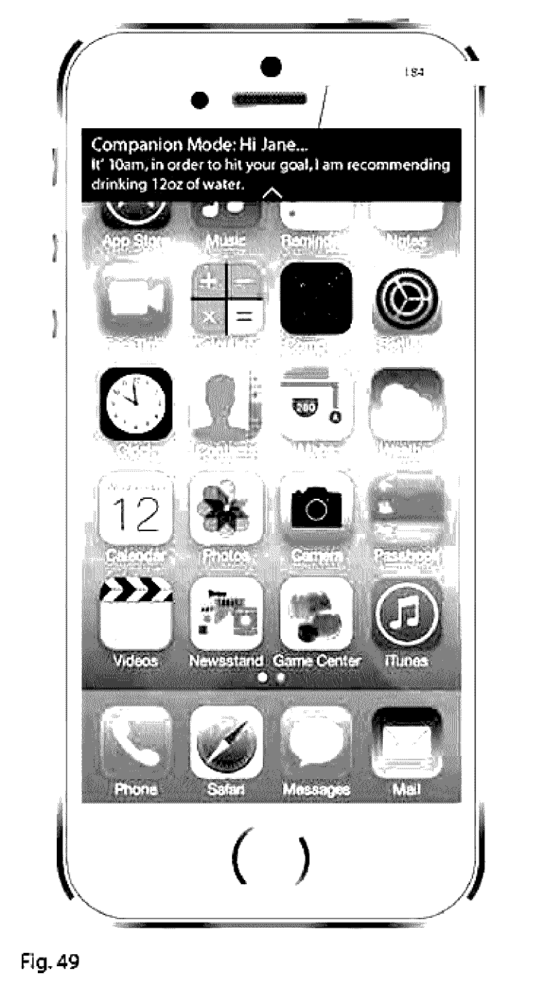

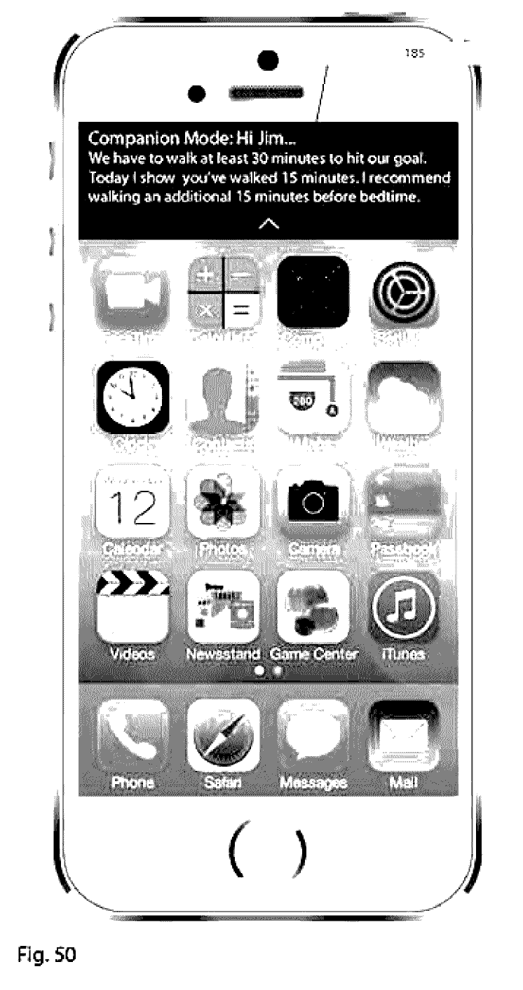

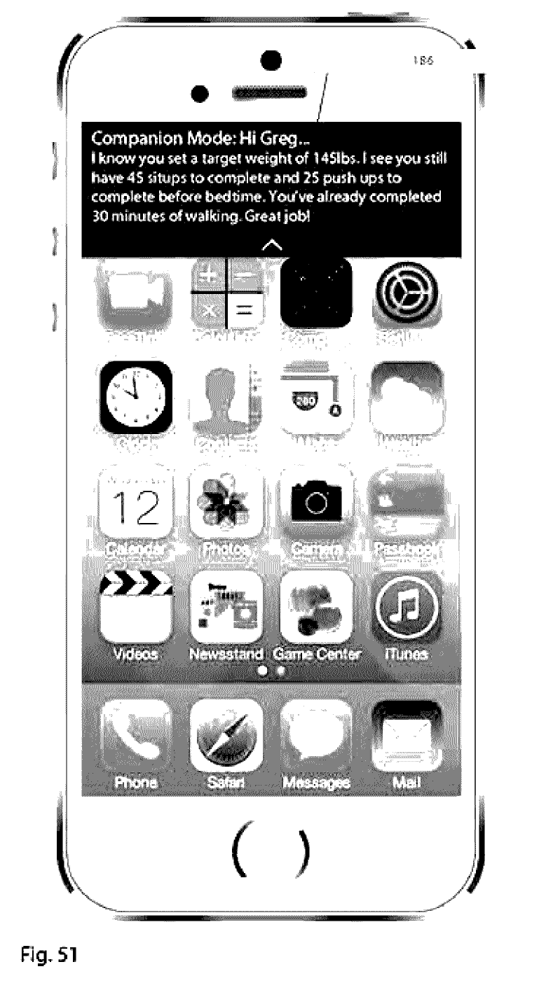

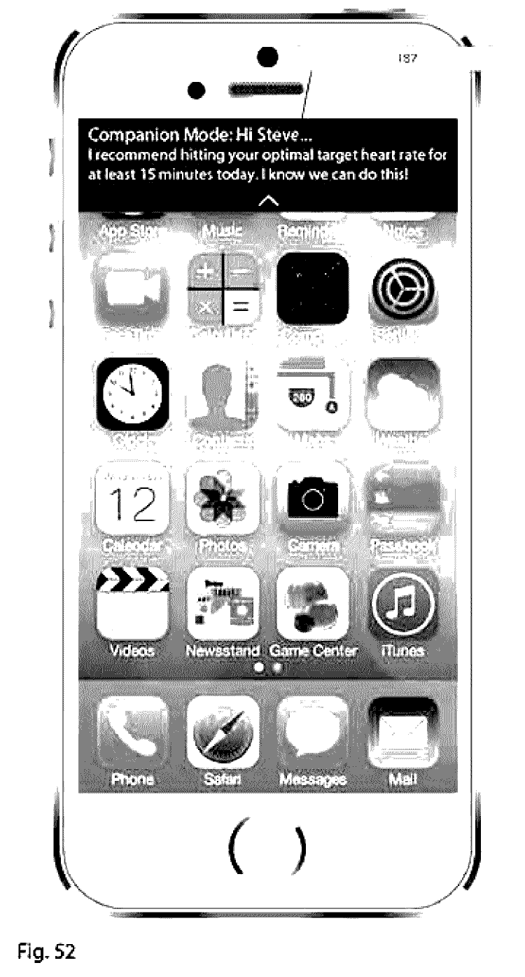

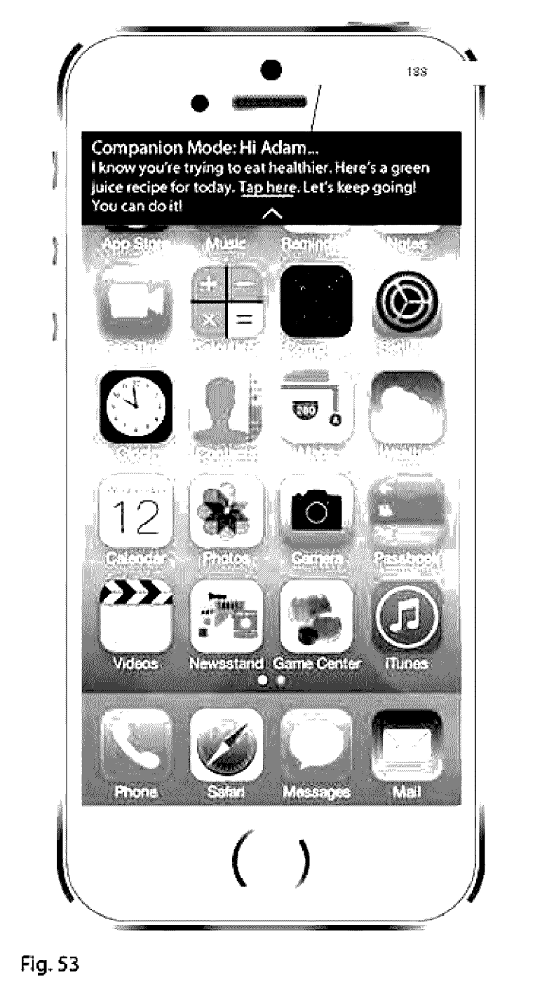

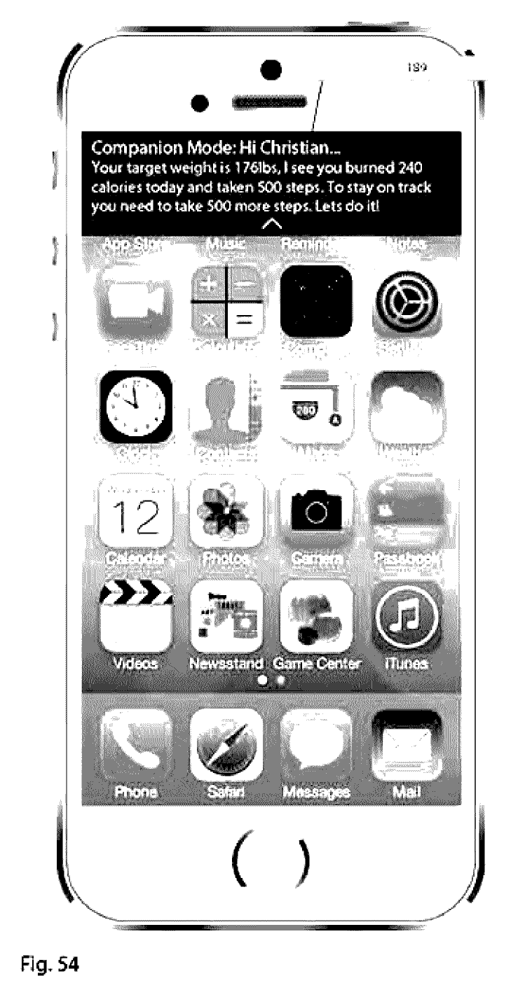

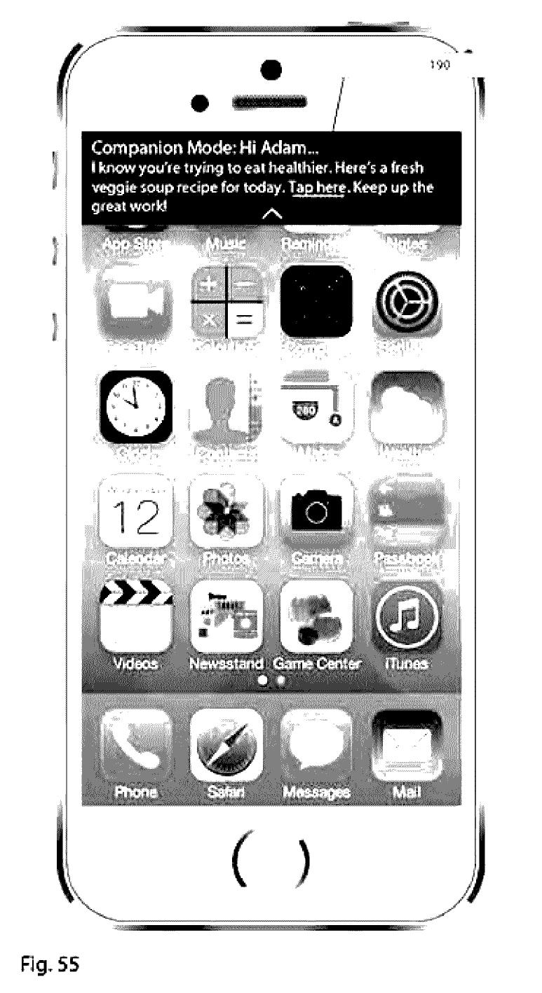

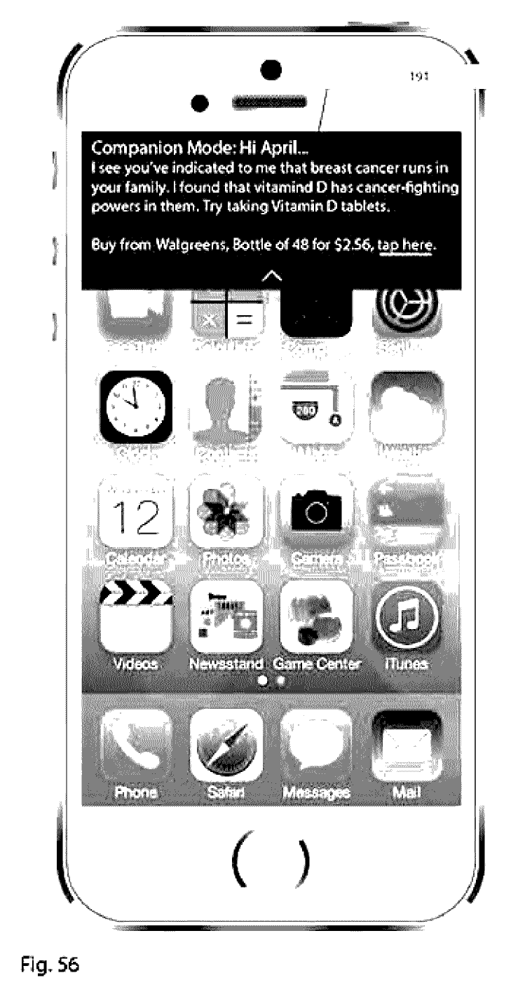

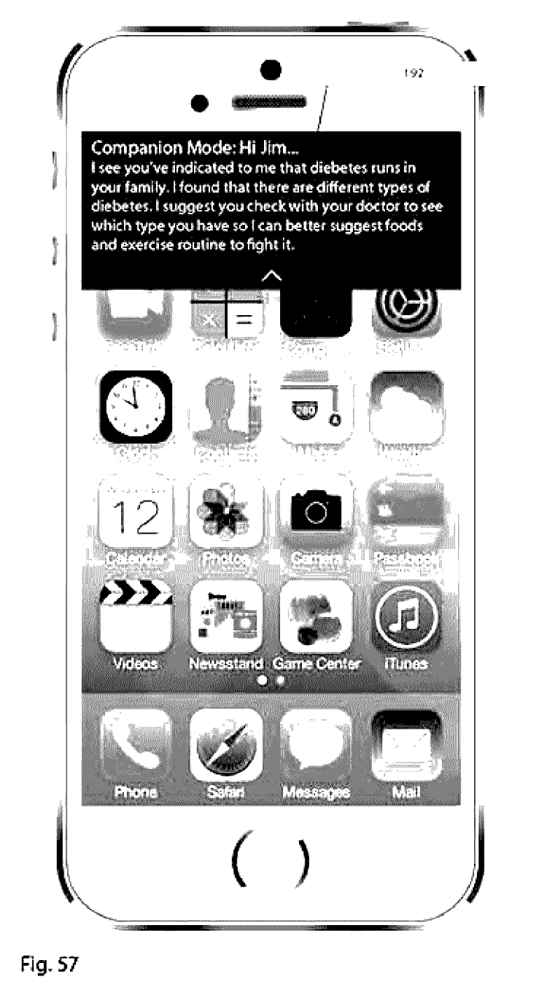

In companion mode, if the wearer was diagnosed with diabetes or any health condition by a medical professional, that information is then selected by the wearer and automatically programmed into the invention. The Qualcomm chip FIG. 2 item 12 will then use suggestive reasoning hierarchy in conjunction with Ai software and provide suggestive alerts to the wearer, SEE FIG. 49, SEE FIG. 50, SEE FIG. 51, SEE FIG. 52, SEE FIG. 53, SEE FIG. 54, SEE FIG. 55, SEE FIG. 56 and SEE FIG. 57.

When the invention detects a specific unhealthy heart rate through the sensor patch FIG. 2 item 26, that's embedded into the invention, it may suggest the wearer to walk at certain times, walk certain distances, and at certain speeds etc. to bring the heart rate up to healthy readings. The invention will then alert the wearer they're back to normal heart rate and working towards strengthening their heart muscle.

The invention may further alert the wearer to eat certain fruits and vegetables in between meals at certain times to maintain sugar levels and keep wearer's metabolism up. Companion mode will also suggest food recipes that are healthy and work to suppress diabetes and other health conditions in an effort to eliminate diabetes or other health conditions depending on the level or type of the disease.

It will provide suggestive exercise routines, foods diets and other information and activity recommendations that work to move the wearer to a healthier lifestyle. Companion mode also encourages the wearer to remain active through alerts. It alerts the wearer to eat small healthy snacks in between meals to speed up the wearer's metabolism and drink more water. This feature aims to target health conditions and disease by alleviating and/or preventing any health complications through heuristic solutions and user controlled interaction.

It will also offer brief educational sentences about the diabetes disease to the wearer over time. It will continue educating the wearer as the wearer continues to interact with the invention. Companion mode will enrich the lives of many people through heuristics, permissions, instructions, suggestions, health information, education, encouragement and features using Qualcomm Neural Processing Unit and Ai software.

The Qualcomm Neuromorphic Chip is a breakthrough in processing chips. It allows for devices to run smoothly and maximize battery life driven experiences. As mobile computing becomes increasingly pervasive, so do expectations of the devices we use and interact with in our everyday lives. Using this chip, devices will be smarter, they will anticipate our needs, and share our perception of the world so we can interact with them more naturally.

As the user interacts and communicates with the invention through selected options, the Qualcomm Neuromorphic Chip will learn about the wearer and continue to work and encourage healthy steps to fighting the diseases, either hereditary or not.

In another example of the invention, a Pedometer is integrated into the microcontroller. The pedometer counts each step a person takes by detecting the motion of the person's hips. Because the distance of each person's step varies, an informal calibration, performed by the user, is required if presentation of the distance covered in a unit of length such as in kilometers or miles is desired, SEE FIG. 61.

Pedometers can be a motivation tool for people wanting to increase their physical activity. The inventions ecosystem allows the pedometer to encourage the wearer be more active, in turn strengthening the wearers health and lowering their risk of serious health problems.

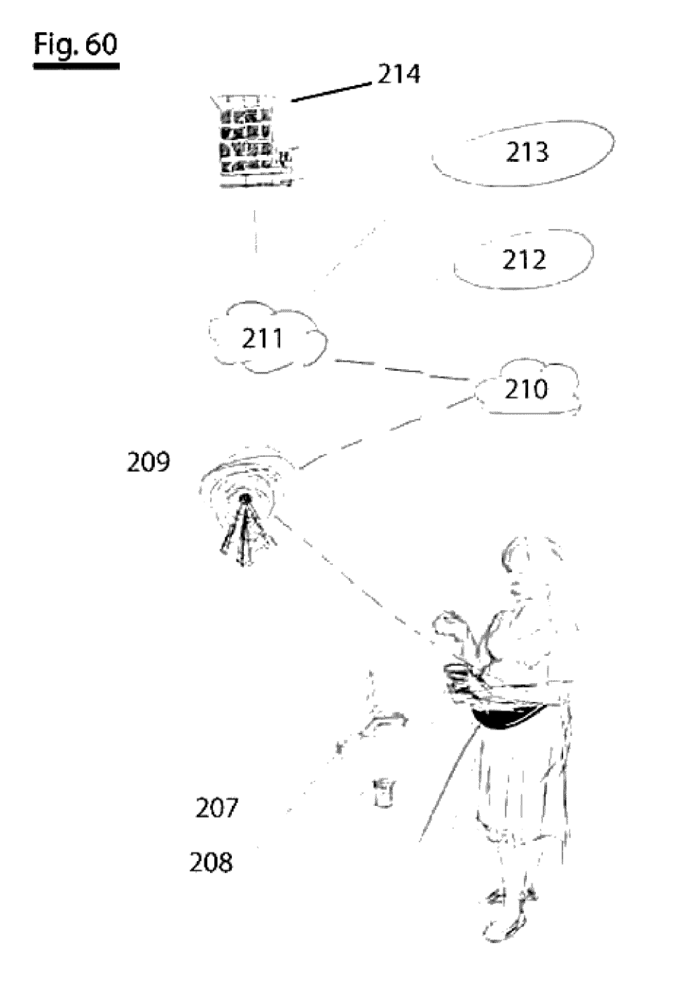

In yet another example of the invention, it's able to store miniaturized finger pricking devices in the detachable medical compartment SEE FIG. 3, ITEM 31. The finger pricking device can be used to draw blood, analyze it and send the results and blood data through the inventions pCell technology SEE FIG. 2, ITEM 30 to local med stations in Walgreens, Rite Aid and Wal-Mart from anywhere making the process easy for the wearer. The invention can also send the same data to hospitals, ER rooms, medical doctors and other medical facilities for quick diagnoses and health action plans, FIG. 60.

In another example of the invention, a food scanner feature is available through the Smartphone app FIG. 2 item 27. By using this feature, the wearer can scan foods at local food markets. The invention will then cross-reference those foods with the wearer's current health condition based on what the wearer input and give the wearer a green light or red if it's healthy or not to eat. This feature makes eating healthy simple for the wearer.

In another example of the invention, a sleep tracker is integrated. The sleep tracker technology measures biomechanics of natural human motion and sleep optimization to bring the best professional medical-class tool to the wearer. The wearer can measure and correlate resting heart rate with sleep quality. This is the most advanced and accurate sleep-cycle alarm solution. It can also use smart alarms for optimal length power-naps and advanced automatic get active alerts.

In yet another example of the invention, the fetus tracking features gives the wearer information regarding the progressive states of each week and month. From inception to week 41, it will allow the wearer to upload files, ultra sound information and provide educational information to ensure the wearer is on track to a healthy delivery.

In yet another example of the invention, the invention can be setup where the wearer is reminded to take a walk at certain times and dates that are selected based on diet and exercise of the wearer. Through these alerts, there are also suggestive ways to get more out of the wearers walk, such as small weights in each hand, speed walking every 5 minutes and other encouraging and health conscious actions.

In another example of the invention, through the Smartphone app FIG. 2 item 27, it will education, recommend, encourage and remind the wearer how many meals to eat per day, what types of snacks to eat, portions, types of fruit and vegetables and recommended daily intake. The purpose of the inventions suggestive reminders is that it will train the brain of the wearer to be conscious of healthy habits, slowly encouraging and moving the wearer to a healthier lifestyle routine.

In another example of the invention, through the Smartphone app FIG. 2 item 27, it will educate, recommend, encourage and remind the wearer to drink water. It will display how many ounces of water per day are recommended and track how many the wearer has drank through interaction with the invention. This will encourage the wearer to drink more water.

In one example of the invention, through the Smartphone app FIG. 2 item 27, there is information about alcohol and tobacco detox classes, education and tips the wearer can do to step away from alcohol and tobacco use. During pregnancy, smoking will contaminate the body including the fetus with over 4,000 chemicals, including cyanide, lead, and at least 60 cancer-causing compounds. The Smartphone app FIG. 2 item 27 educates, promotes and encourages the wearer to quit drinking and smoking.

In one example of the invention, the Smartphone app FIG. 2 item 27 provides a recommended green juice list that targets the wearer's hereditary disease or health conditions. The Smartphone app FIG. 2 item 27 will allow the user to tap on different types of ingredients and the health benefits will display, showing the wearer how it will impact their life and vitals. This further broadens the invention, and solidifies that it's not replicable without copying the invention in some form or fashion.

In another example of the invention, the Smartphone app FIG. 2 item 27 provides specific or general diet plans and/or recipes that help encourage a healthy eating lifestyle. This feature can be changed based on the wearers input as to their current health condition and the invention will cater to the wearer. The invention will specifically calculate what foods would target the health condition.

In another example of the invention, the Smartphone app FIG. 2 item 27 will provide hundreds of types of stretches, workout routines and both pre/post workout suggestions. The invention is designed to place the wearer in a routine of workouts that help the wearer become healthier.

In another example of the invention, the Smartphone app FIG. 2 item 27 allows the wearer to use the calendar and baby movement feature. This feature provides the wearer to record movement, track progress and count the kicks and other movements to ensure a healthy development of the fetus. Most pregnant women start to feel their baby move around 18 to 20 weeks of pregnancy.

Initially, these movements vary a lot, but they settle into a familiar pattern enabling you to monitor your baby's movements. A reduction in movements can mean that the baby is not well in the womb and a very small proportion of these babies need to be delivered urgently. The Smartphone app FIG. 2 item 27 helps the wearer become fully aware of what to look for to ensure both her and her fetuses health.

In yet another example of the invention, the Smartphone app FIG. 2 item 27 allows the wearer to calculate the wearer baby's due date. Usually wearers are not sure when the wearers baby is due, when the conception date was, how many weeks pregnant, when the end of the first trimester is, the Smartphone app FIG. 2 item 27 solves this problem, giving the wearer peace of mind relieving stress throughout pregnancy.

In another example of the invention, through the Smartphone app FIG. 2 item 27, the precious moments feature allows the wearer to upload progression photos, baby bump photos, first heartbeat, ultra-sound photos and other sentimental and milestones for the pregnancy. This can be uploaded and connected with Facebook and other social media's or sent to be printed out as a scrap book, photos or postcards to family and friends.

In another example of the invention, through the Smartphone app FIG. 2 item 27, it provides vital prenatal checkup reminders, appointment times and dates. This is a very crucial feature. In this feature, the wearer is instructed to track, note and educate about each doctor's appointment. Throughout the pregnancy, the wearer is also guided to conduct an extensive fact-finding mission to learn everything about the wearer's family medical history.

After the wearer inputs the findings of hereditary diseases, the Smartphone app FIG. 2 item 27 reconfigures to offer exercise routines like yoga and speed walking that targets obesity. It further offers foods like green juice which consists of fruits and vegetables that target cancers. It will continue to offer diets that help maintain a healthy weight such as recipes and portion control ideas, as well as a plan of action to suppress the specific diseases selected.

When the wearer begins to eat those disease targeting foods, through a physiological connection to the fetus, the fetus is memorizing those same flavor pallets through taste memory that's being transferred in the amniotic fluid from the mother. This gives the fetus a profound advantage over an inevitable hereditary disease by preferring foods that target the disease to regress it instead of progressing it.

In yet another example of the invention, the Smartphone app FIG. 2 item 27 provides a list of OB/GYN recommended to-do list. The feature educates the wearer about various things the wearer would need to do to ensure a healthy pregnancy. These include, but not limited to, prenatal testing, ultrasound, medications and home stretch classes.

The features included in the invention are boundless and will always evolve through time, the list of other features are the following, but not limited to, pre/post pregnancy item list, breastfeeding initiation, duration, and exclusivity information, menstruation and ovulation calendar to track menstrual history, body temp, avg. cycle length pre pregnancy, physician alerts and tips, prenatal vitamin recommendations and intake reminders, baby name list, pregnancy journals, weight gain tracker, awareness list of early detection and signs of pregnancy problems, check-in with pregnant friends feature, digital fitness coach, pregnancy safe activities list and belly photo tracking.

In yet another example of the invention, the Smartphone app FIG. 2 item 27 encourages a stress free pregnancy for the wearer. To achieve this, the Smartphone app FIG. 2 item 27 gives the wearer the opportunity to plan for the future during the sign up process by offering the following, but not limited to, baby budget manager, prenatal health insurance manager and Baby College fund manager through various financial institutions.

In one example of the invention, there is an option to download pre-birth audio. This is defined as audio that is played to a fetus at any stage in the pregnancy for purposes of education

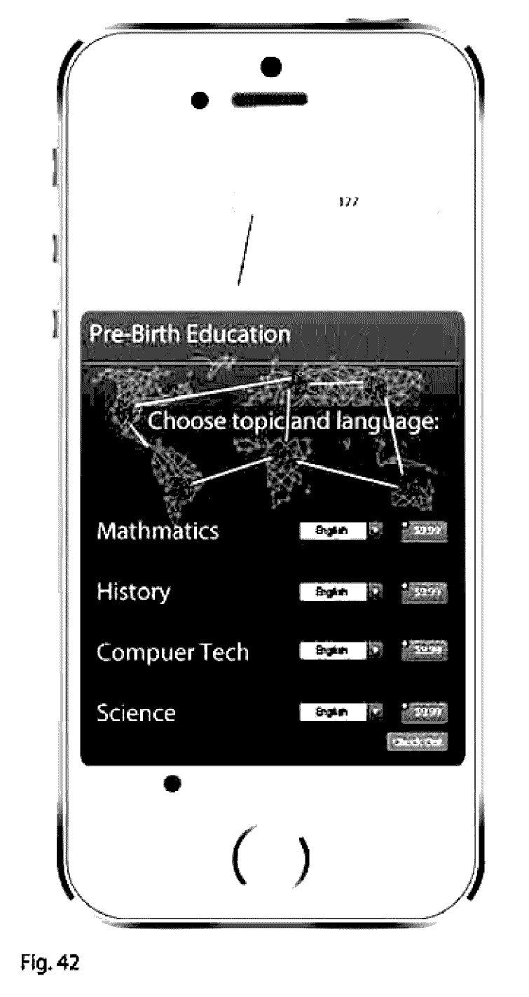

The audio can be multi-cultural and in multi-language and is able to be downloaded on iTunes and the Marketplace, SEE FIG. 42, for sole purpose of pre-birth education about life in the following, but not limited fields:

Aerospace Engineering, Africana Studies, American Indian Studies, American Studies, Animal Science, Agriculture and Life Science, Animal Science, Anthropology, Applied Economics and Management, Agriculture and Life Sciences, Applied Mathematics, Applied Physics, Archaeology, Architecture, Architectural Science, Architecture Design, History of Architecture and Urban Development, Art, Asian Literature, Religion and Culture, Asian Studies, Astronomy and Space Sciences, Astronomy, Atmospheric Science Biochemistry, Molecular and Cell Biology, Biological and Environmental Engineering,

Additionally the invention gives a platform to the wearer to play Cultural tolerance educational audio. This means the audio can be played in hundreds of languages and cultural understanding, but not limited to the following:

Afghans, Albanians, Algerians, Americans, Andorrans, Armenians, Australians, Brazilians, Bretons, Canadians, English, Spanish, Germans, French, Hong Kong, Vietnamese, Arabs, Koreans, Zimbabweans and others.

There are endless examples as to what the invention can do in terms of playing audio to a fetus. This holds true scientifically as the auditory system is one of the first systems to develop. Studies have proven that playing Mozart stimulates the same neurons in the brain that play a key role in mathematical ability. But that doesn't have to be the limit for an unborn baby which my invention serves that purpose.

As an example, if the expectant mother wants her unborn child to have a tolerance for other cultures and nationalities, she can download pre-birth educational audio from the invention, the invention item 27, and play available audio. It can be about the ABC's in form of a song, rhythms, carols, orchestrated and others in any particular language so the fetus can recognize the same form of sound when it's born and have an appreciation or connection to the language and culture making our human race even more interconnected.

To support my claim that fetuses can learn pre-birth, a reference is listed below. The reference shows live ultra-sound video of a 14-week unborn baby “clapping along” as the expectant mother and her physician sing “If You're Happy and You Know It,” as the husband films. This is an incredible part of the invention and further proves the invention is focused on health, education and world open-mindedness.

### SUMMARY OF INVENTION AND ADVANTAGES IN MORE DETAIL OF ALTERNATIVE FASHION

When using the invention in a different fashion, style of wear and other methods, below is a listed in various industries, but not limited to:

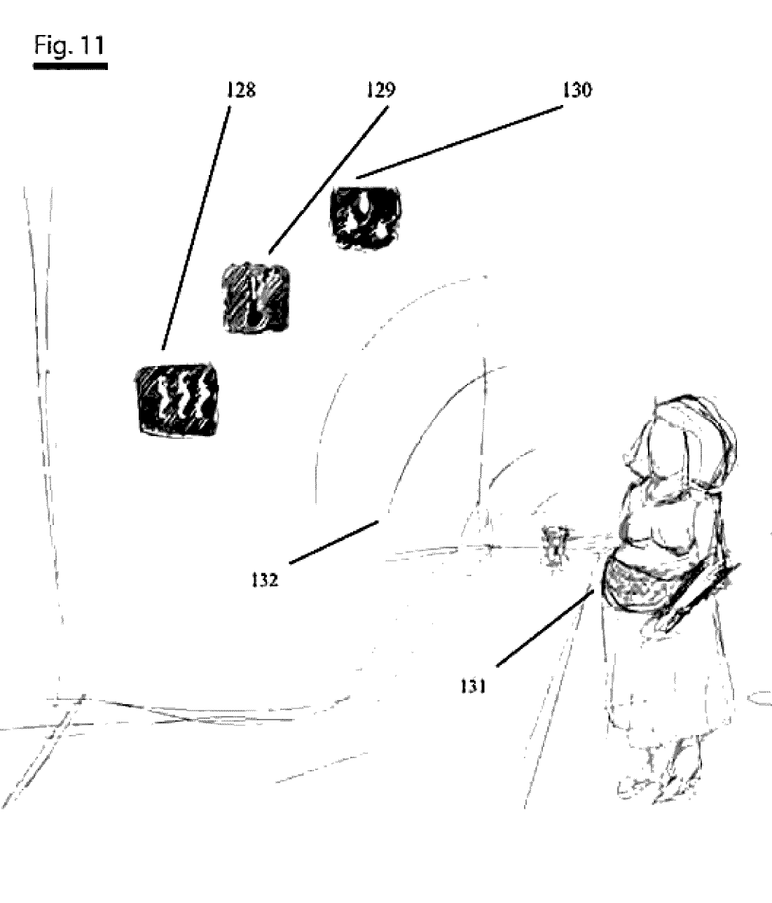

### Housing—See FIG. 11

The following safety detection and alerts can be used in the invention which will connect using Bluetooth enabled devices such as carbon monoxide, temperature, humidity hardware. The hardware installed into the house to provide the wearer with alerts to save their life.

### Contractors

Electricians can use the invention in which it can assist with data or answer questions as the contractor is working while offering back support.

### Fitness

Fitness enthusiasts can use the invention to monitor their fitness progress, connect to videos and provide back support all at the same time.

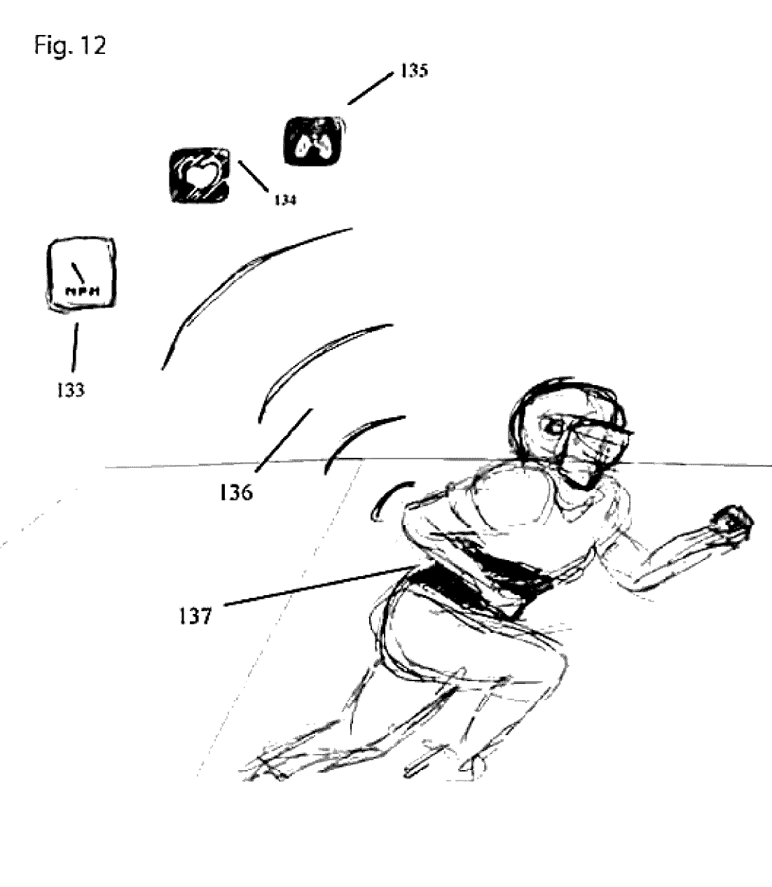

### Sports—See FIG. 12

The invention can monitor heart rates and rate variability, breathing rate, respiration volume for professional athletes; NFL track yards gained, speed of runner, monitor vitals for improvement of feedback in real time.

### Universities

Professors who lecture can transmit all of their course material to the projector using the invention while providing back support while the professor stands for hours at a time.

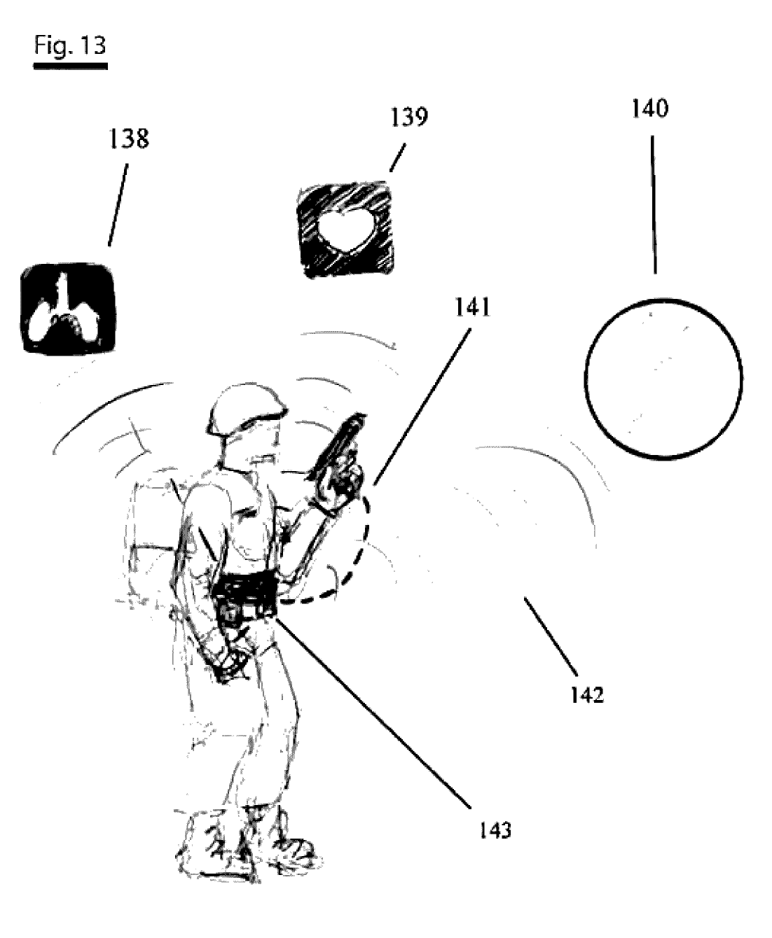

### Military—See FIG. 13

The invention can capture and store life data, GPS, live health monitor, sync with weapons for the U.S. military. SEE FIG. 71, the U.S. soldier wearing the invention connects to the cell tower, smart tank connection, smart tank, when heart rate, blood pressure goes up suddenly, the invention will signal the smart tank to go weapons active and reads the soldiers body so the soldier can react more efficiently in combat.

### Restaurant

Waitresses and waiters can use the invention to transmit orders that are sent to the chef, reservations and others features offering more efficiency to the business.

### Night Clubs

Bouncers can use the invention to track VIP rosters, payments through Apple Pay, ID verification, reservations and other features and functionalities.

### Auto Repair

Mechanics and technicians can use the invention for back support while downloading data from the vehicle to detect a diagnostic, send to the consumer the information through the inventions Smartphone app FIG. 2 item 27 so the consumer knows exactly what is wrong with their vehicle.

### Space/NASA

Astronauts can use the invention to provide an array of data, health monitoring and storage capacity.

### Nascar

The invention can track the health vitals of the driver while racing applications are endless.

### Auto

The invention will automatically open doors, start vehicles with preset settings that are programmed using the inventions Smartphone app FIG. 2 item 27. The invention can also detect if your pulse drops too low where you lose consciousness and instructs the vehicle to pull to the side of the road safely using the automatable technologies.

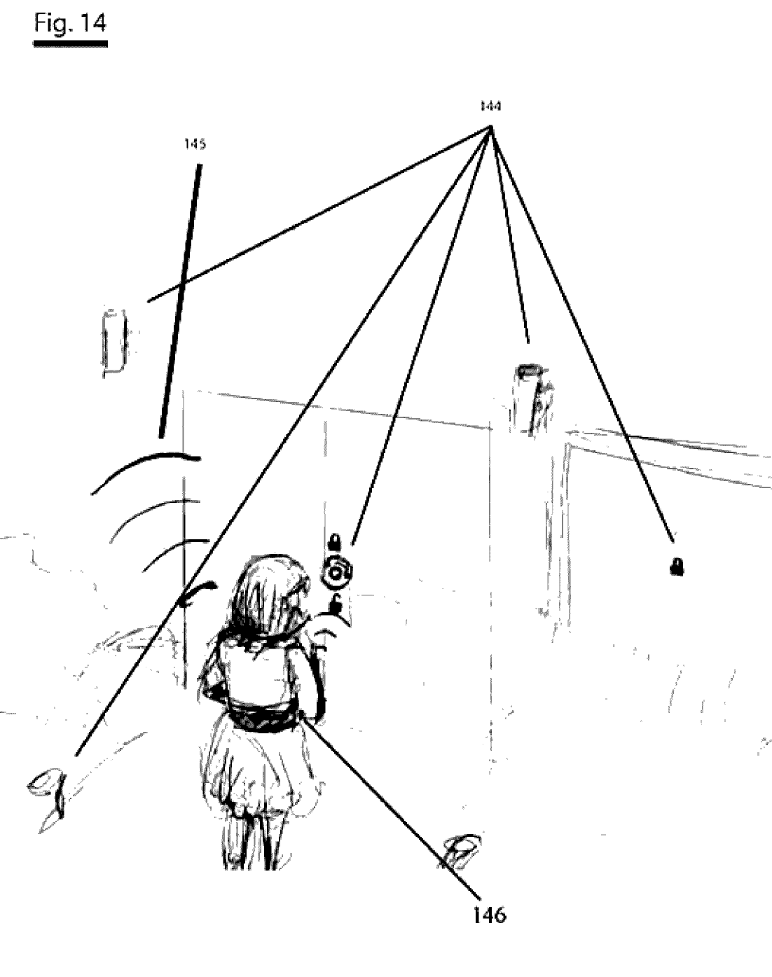

### Home Security—See FIG. 14

The invention can be altered to detect when you come home and open doors, set temperature through preset settings that are programmed using the inventions Smartphone app FIG. 2 item 27 and arm/disarm the alarm system, turn on/off lights as you walk in the room and other features using technology through the home security technologies in conjunction with my invention.

### Smart Car—See FIG. 63

The invention can be altered to connect to smart cars and automatically open doors, set A/C, seat adjustment and other preferences.

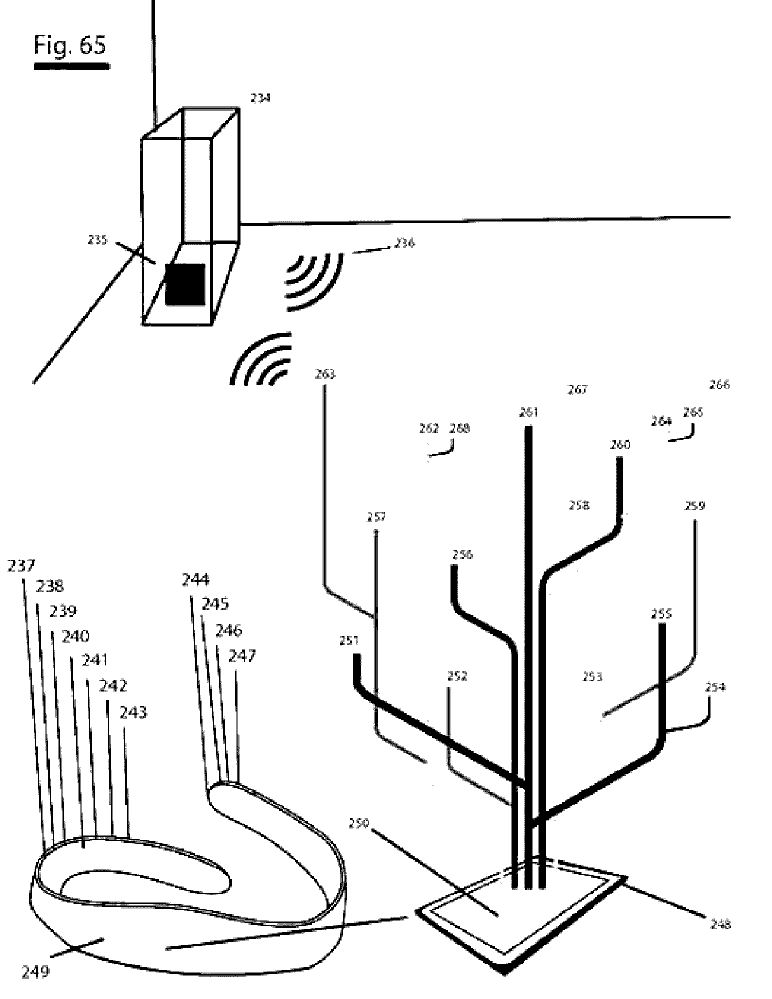

### Smart Clean Home Batteries Management—See FIG. 65

The invention can be altered to automatically charge using a smart battery home power system that is being sold by Tesla. This allows the invention to run as the brain of the smart home.

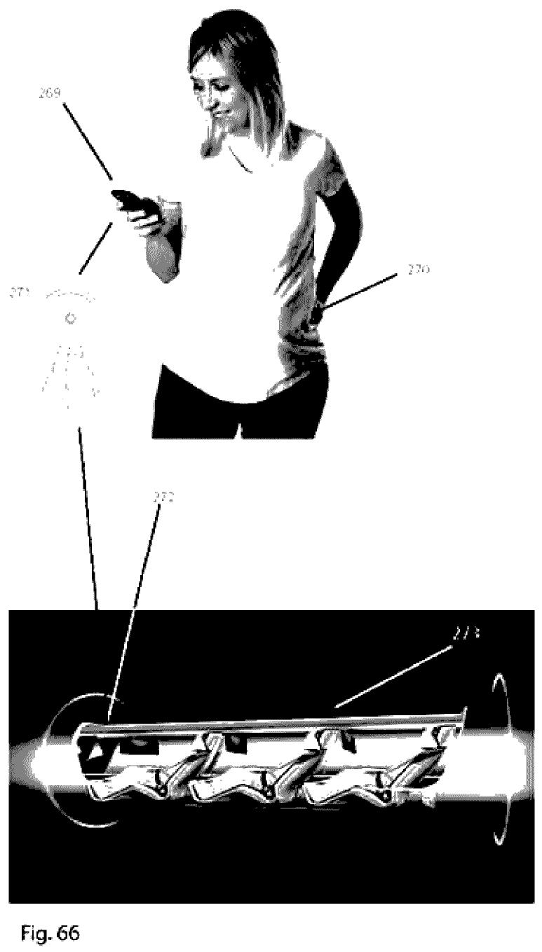

### Smart Train Travel—See FIG. 66

The invention can be altered to know where the wearer is traveling to, the cost and allow the wearer to charge it using the Smartphone App as part of the invention as well as other preferences that is set in the invention.

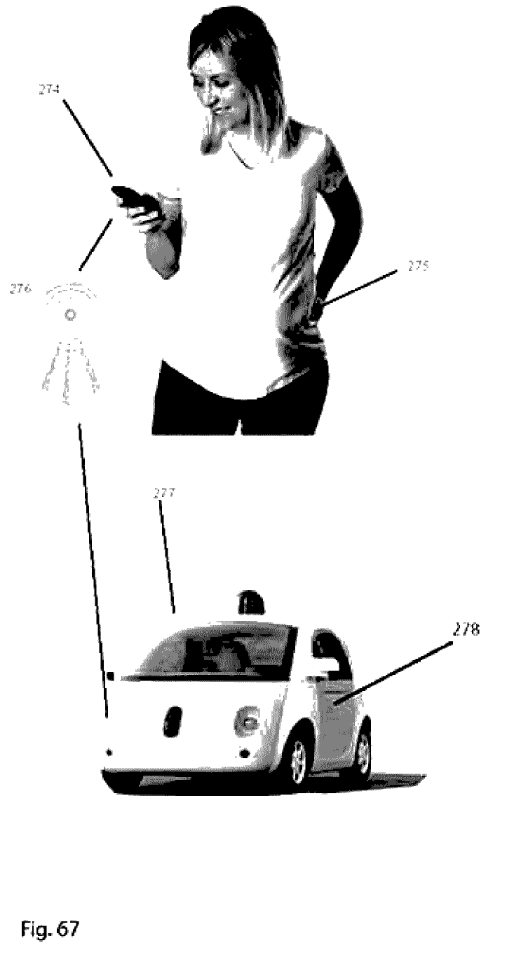

### Self Driving Cars—See FIG. 67

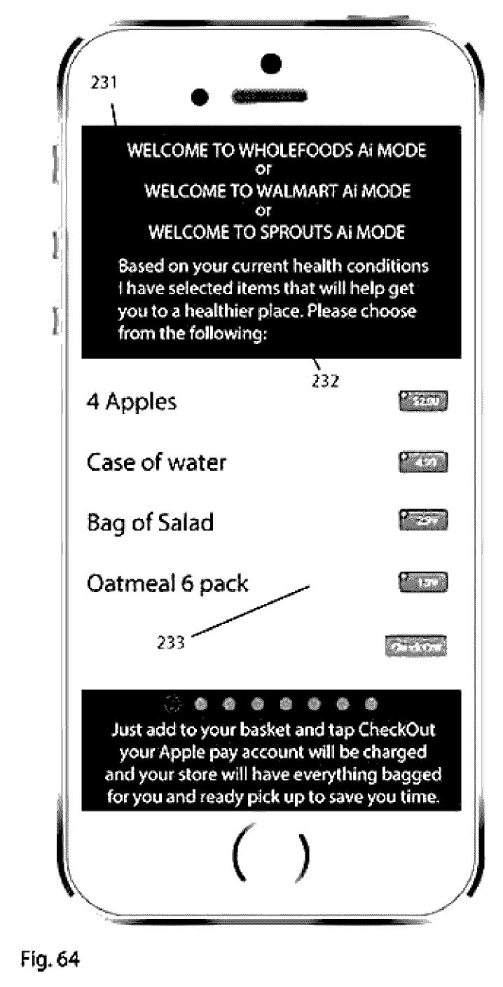

The invention can be altered to automatically drive where you made reservations using the invention, or grocery store where you purchased foods based on what's healthy for you see FIG. 64.

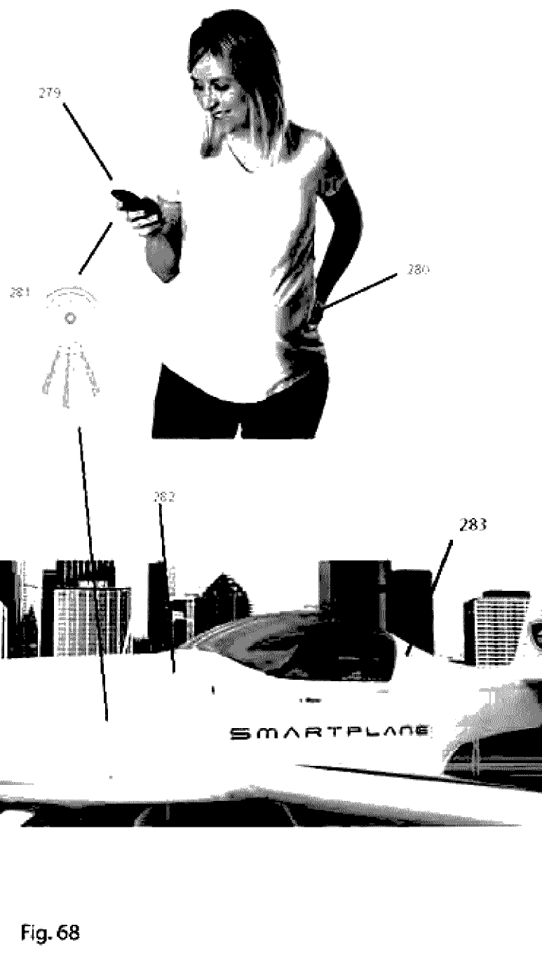

### Smart Planes—See FIG. 68

The invention can be altered to know where your hotel is in what country and charge you through the inventions Smartphone app and fly you there with your preferences.

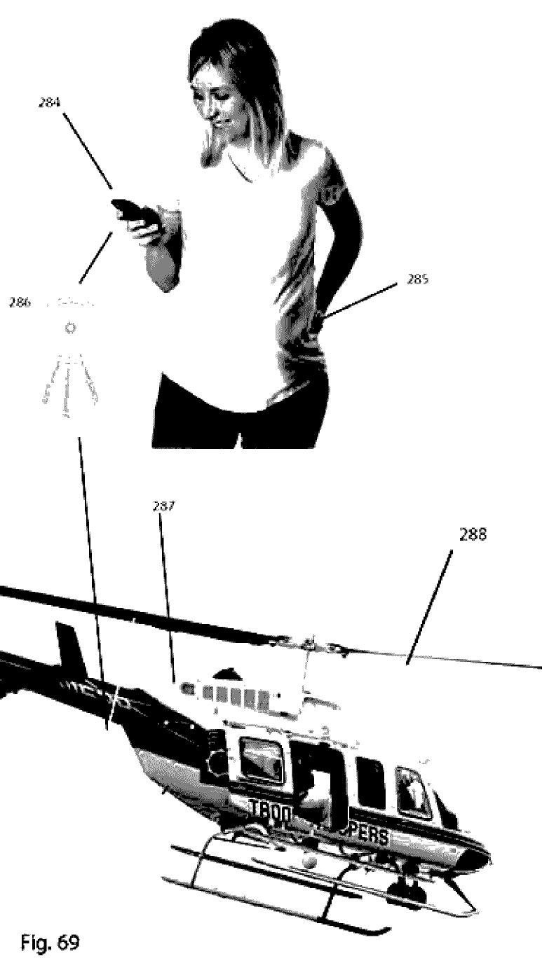

### Smart Helicopters—See FIG. 69

The invention can be altered to detect through the Smartphone app as part of the invention to fly you where you need to go and the total cost and preferences.

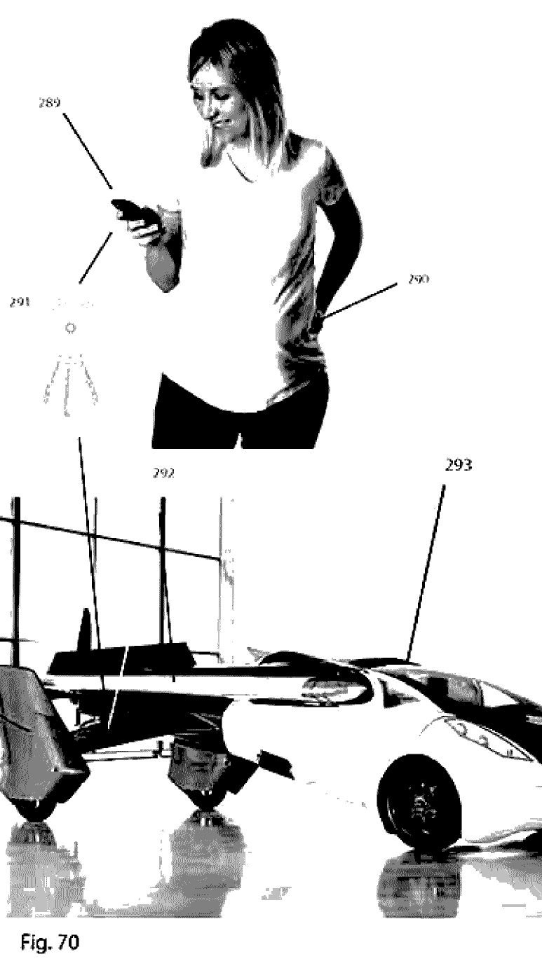

### Flying Cars—See FIG. 70

The invention can be altered to fly you to a family members home based on the Smartphone app which is part of the invention or a shopping mall, parts, recreation and others.

### BRIEF DESCRIPTION OF THE DRAWINGS

FIG. 1 is a perspective view of the back of the invention, displaying items 1-9;
Item—1—Cloth
Item—2—E-textile
Item—3—Copper Oxide Fibers
Item—4—Shape-Memory and Polymers
Item—5—Velcro
Item—6—Elastic Material
Item—7—Peruvian pima Cotton Liner
Item—8—Embedded sensors
Item—9—Elastic Thread or something strong to last longer

FIG. 2 is a perspective view of the back of the invention, displaying items 11-30;
Item—11—Microcontroller Circuit Board with bios and firmware
Item—12—Qualcomm Neuromorphic Chip
Item—13—Memory Chip
Item—14—Class II Bluetooth Chip
Item—15—Microphone, Sound Decoder and Codec and Amplifier Chip
Item—16—Auto Length Accelerometer-Based Board
Item—17—Altimeter Board
Item—18—6-Axis Accelerometer Board
Item—19—Gyroscope Board
Item—20—Rechargeable Pure Aluminum-Ion Battery with Integrated Power-Saving Accelerometers Board
Item—21—Health Sensors
Item—22—Calorie Counter Board
Item—23—Pedometer Board
Item—24—Bluetooth® Smart Technology Software
Item—25—Detachable Bluetooth Speakers
Item—26—Sensor Patch
Item—27—Smartphone App
Item—28—GPS Nano-Spider technology and accelerometer boards
Item—29—Small GPS Wrist Band
Item—30—pCell Chip and Board

FIG. 3 is a perspective view of the back of the invention, displaying item 31;
Item—31—Detachable and Adjustable Medical Compartments

FIG. 4 is a perspective view of the back of the invention, displaying items 32-49;
Item—32—Stationary Layer
Item—33—Detachable Layer with female connecting rod loop
Item—34—Types of Material
Item—35-43—Detachable Layer Shape Options with various types of material with female connecting rod loop
Item—44-49—Types of material Options

FIG. 5 is a perspective view of the back of the invention, displaying items 50-72;
Item—50—Casing with male connecting rod loop
Item—51—Microcontroller completely assembled
Item—52—Stationary Layer
Item—53—Microcontroller Circuit Board with bios and firmware
Item—54—Qualcomm Neuromorphic Chip
Item—55—Memory Chip
Item—56—Class II Bluetooth Chip
Item—57—Microphone, Sound Decoder and Codec and Amplifier Chip
Item—58—Auto Length Accelerometer-Based Board
Item—59—Altimeter Board
Item—60—6-Axis Accelerometer Board
Item—61—Gyroscope Board
Item—62—Rechargeable Pure Aluminum-Ion Battery with Integrated Power-Saving Accelerometers Board
Item—63—Health Sensors
Item—64—Calorie Counter Board
Item—65—Pedometer Board
Item—66—Bluetooth® Smart Technology Software
Item—67—Detachable Bluetooth Speakers
Item—68—Sensor Patch
Item—69—Smartphone App
Item—70—GPS Nano-Spider technology and accelerometer boards
Item—71—Small GPS Wrist Band
Item—72—pCell Chip and Board

FIG. 6 is a perspective view of the Cloud server setup, displaying items 73-86;
Item—73—Cloud
Item—74—Cloud Service Queue
Item—75—Cloud Infrastructure Database and App Connection
Item—76—Cloud Platform Web Frontend and Backend
Item—77—Proprietary Algorithm
Item—78—Cloud Storage
Item—79—WWW
Item—80—ISP
Item—81—Router or Modem
Item—82—Cell Towers
Item—83—Mobile
Item—84—Notebook
Item—85—Invention
Item—86—PC

FIG. 7 is a perspective view of user provided Smartphone and all hardware components, displaying items 87-103;
Item—87—Power System
Item—88—Communication Module
Item—89—External Port
Item—90—Peripherals Interface
Item—91—Processor (s)
Item—92—RAM
Item—93—Display Controller
Item—94—Optical Sensor (s) Controller
Item—95—I/O Subsystem
Item—96—Other Input Controller (s)
Item—97—Touch Sensitive Display System
Item—98—Optical Sensor (s)
Item—99—Other Input Control Devices
Item—100—Camera Module
Item—101—Telephone Module
Item—102—Graphic Module
Item—103—Contact/Motion Module

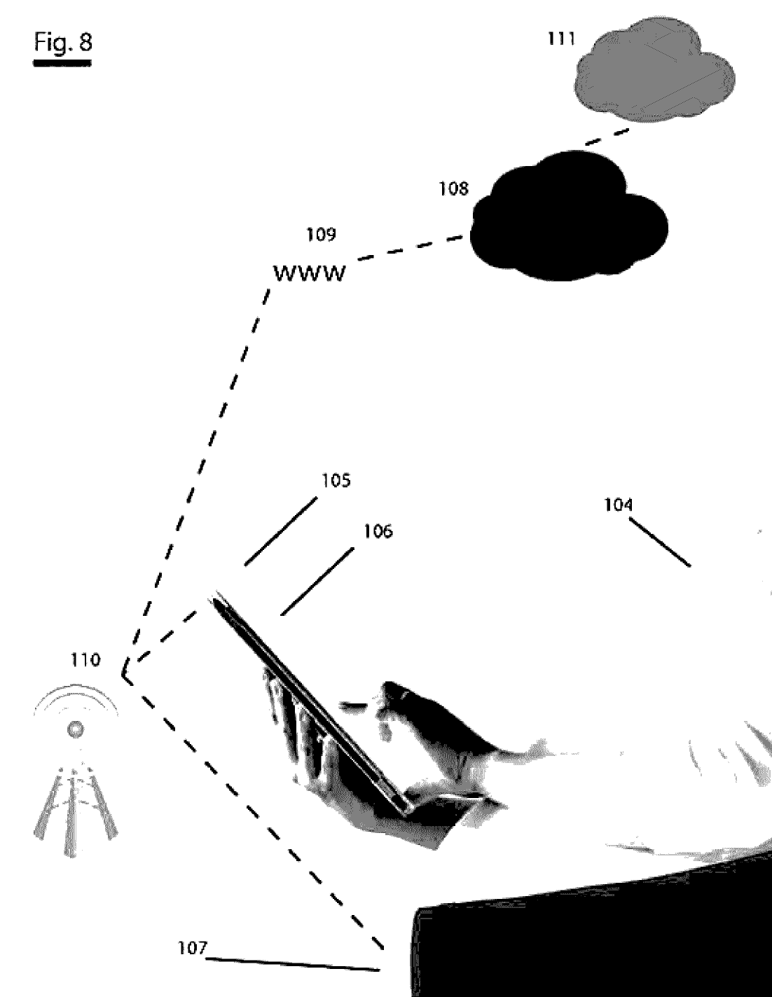

FIG. 8 perspective view of how the entire invention works in the ecosystem, all items, figs displaying items 104-111;
Item—104—Wearer
Item—105—Smartphone
Item—106—Invention Smartphone App
Item—107—Invention Prenatal Belt
Item—108—Invention Cloud
Item—109—Internet
Item—110—Cell Tower
Item—111—Cloud Server and Proprietary Algorithm

FIG. 9 perspective view of detachable shaped material connecting the invention through a hole and loop, displaying items 112-114;
Item—112—Detachable Layer Shape Options with various types of material with female connecting rod loop
Item—113—Stationary Layer
Item—114—Casing with male connecting rod loop

FIG. 10 perspective view of the seconds board with additional chips for more medical device features, displaying items 115-127;
Item—115—Patient Position Sensor
Item—116—Glucometer Sensor
Item—117—Body Temperature Sensor
Item—118—Blood Pressure Sensor (Sphygmomanometer) V2.0
Item—119—Pulse and Oxygen in Blood Sensor (SPO2)
Item—120—Airflow Sensor (Breathing)
Item—121—Galvanic Skin Response Sensor (GSR-Sweating)
Item—122—Electrocardiogram Sensor (ECG)
Item—123—Electromyography Sensor (EMG)
Item—124—Muscle Sensor v3
Item—125—Casing with male connecting rod loop
Item—126—Microcontroller completely assembled
Item—127—Stationary Layer

FIG. 11 perspective view of invention working in conjunction with home alarm system to detect danger displaying items 128-132;
Item—128—Carbon monoxide
Item—129—Temperature
Item—130—Humidity hardware
item—131—Invention Prenatal Belt
Item—132—Bluetooth

FIG. 12 perspective view of sports being able to monitor improvements on players displaying items 133-137;
Item—133—Speed in MPH
Item—134—Respiration
Item—135—Breathing rate
Item—136—Bluetooth
Item—137—Invention Prenatal belt

FIG. 13 perspective view of the military using it for GPS location, vitals and weapon sync based on conditions working in conjunction with the invention displaying items 138-143;
Item—138—Breathing Rate
Item—139—Respiration
Item—140—GPS
Item—141—Weapon sync
Item—142—pCell or 5G Wireless
Item—143—Invention Prenatal Belt

FIG. 14 perspective view of home alarm system automatically locking and unlocking working conjunction with the invention displaying items 144-146;
Item—144—Home Security System
Item—145—Bluetooth
Item—146—Invention Prenatal Belt

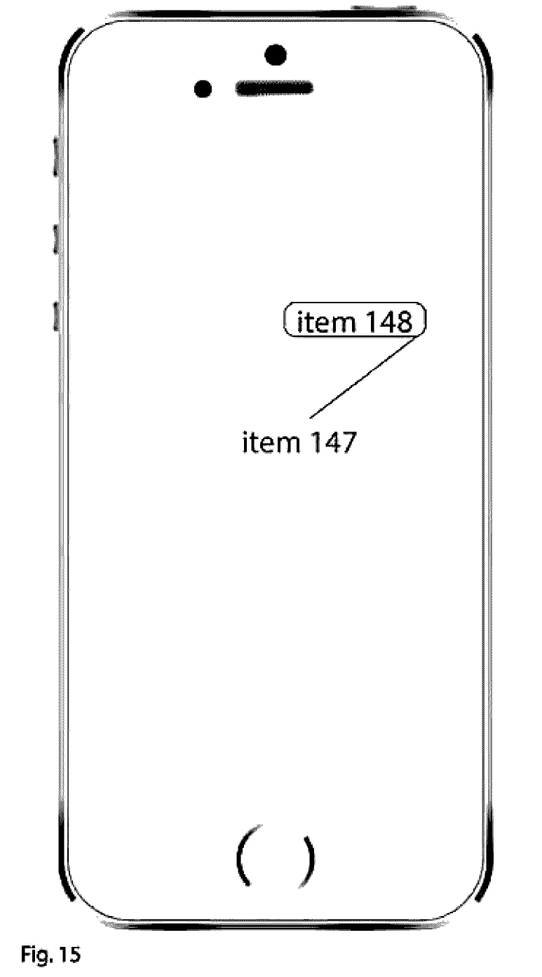

FIG. 15 perspective view of App launch screen displaying items 147-148, this is the main screen you launch when the invention is on and ready to use;
Item—147—Company Logo
Item—148—Start Up Screen

FIG. 16 perspective view of App Registration screen of the app displaying item 148, this is the registration screen where you can place your name, age, DOB, address and other information so the Smartphone app can learn the wearer and provide a more comprehensive and seamless experience;
Item—148—Registration of detailed information of the wearer

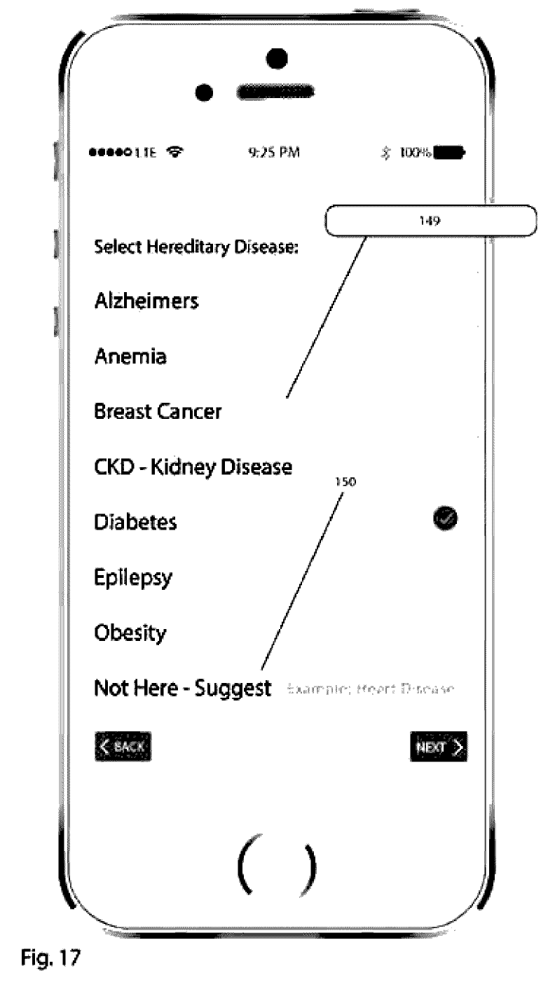

FIG. 17 perspective view of App option to select hereditary disease screen displaying items 149-150, this screen is vital and determines what disease the wearer has and offers suggestive habits, actions and foods to eat to prevent, suppress or cure such as diabetes, heart disease or cancer;
Item—149—Choosing hereditary disease
Item—150—Enter disease

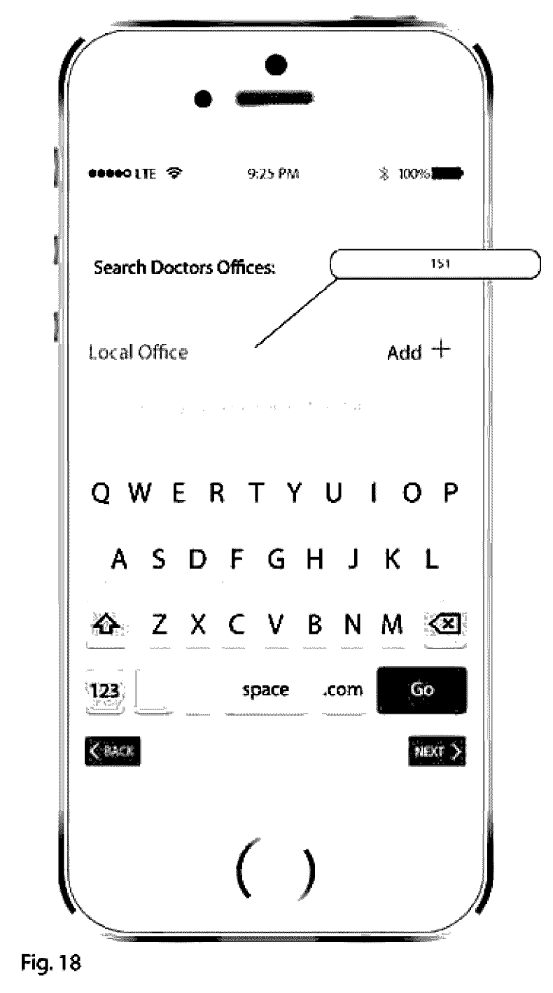

FIG. 18 perspective view of App selecting a doctor's office screen displaying item 151, this allows medical offices and the wearer to connect as well as being able to share their health information so they don't have to fill out long forms or asked repeated questions;
Item—151—Choosing a local medical doctors office

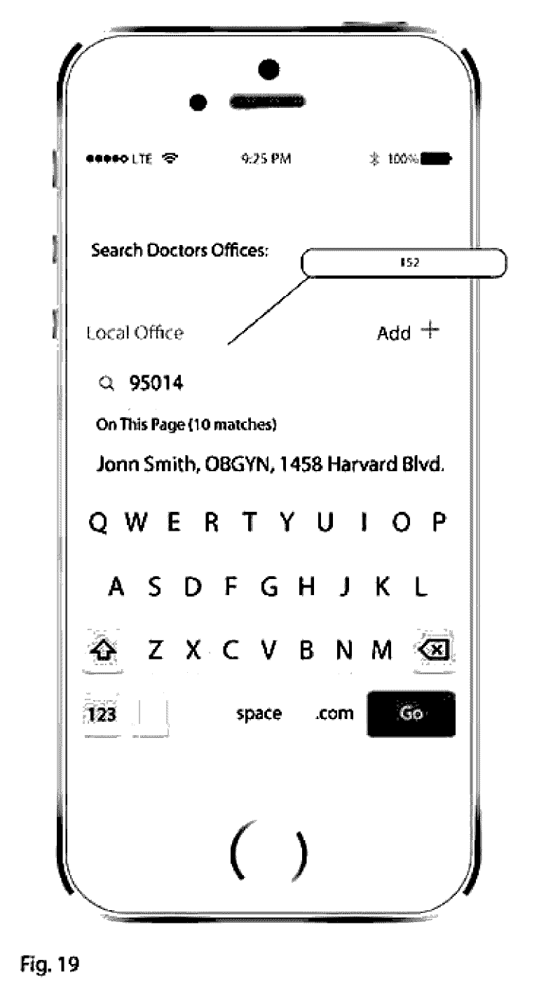

FIG. 19 perspective view of App selecting a doctor's office screen continued displaying item 152;
Item—152—Choosing a local medical doctors office continued

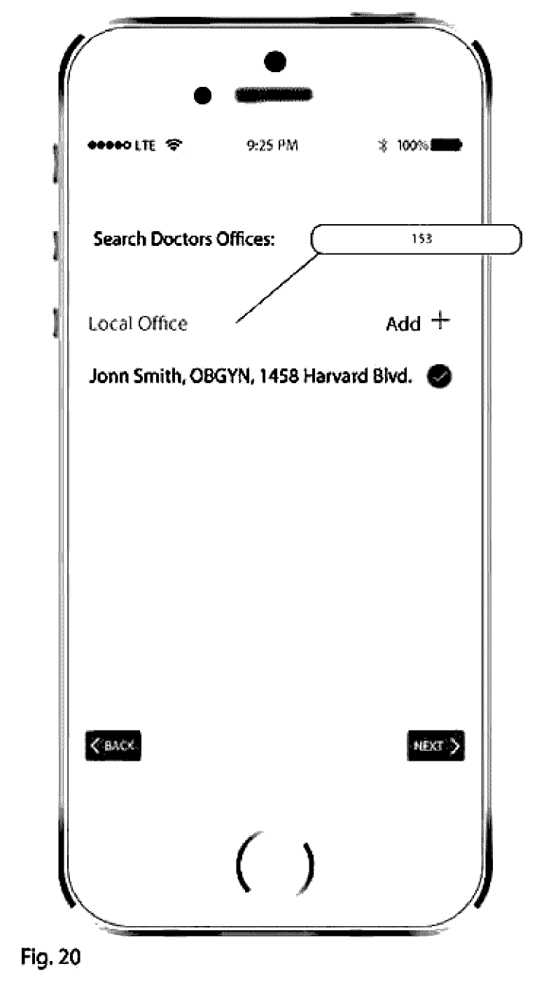

FIG. 20 perspective view of App selecting a doctor's office screen continued displaying item 153;
Item—153—Choosing a local medical doctors office continued

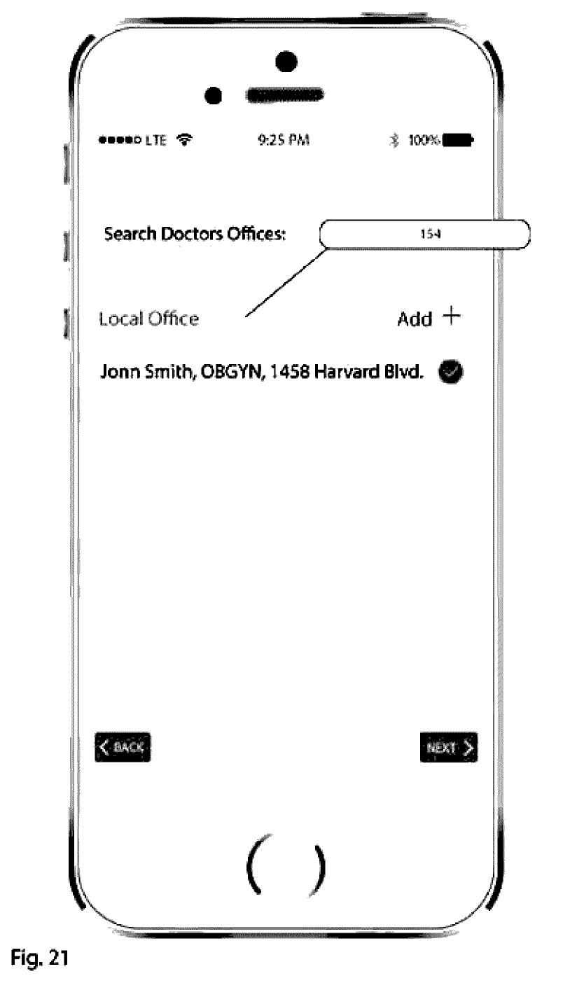

FIG. 21 perspective view of App selecting a doctor's office screen continued displaying item 154;
Item—154—Home Security System

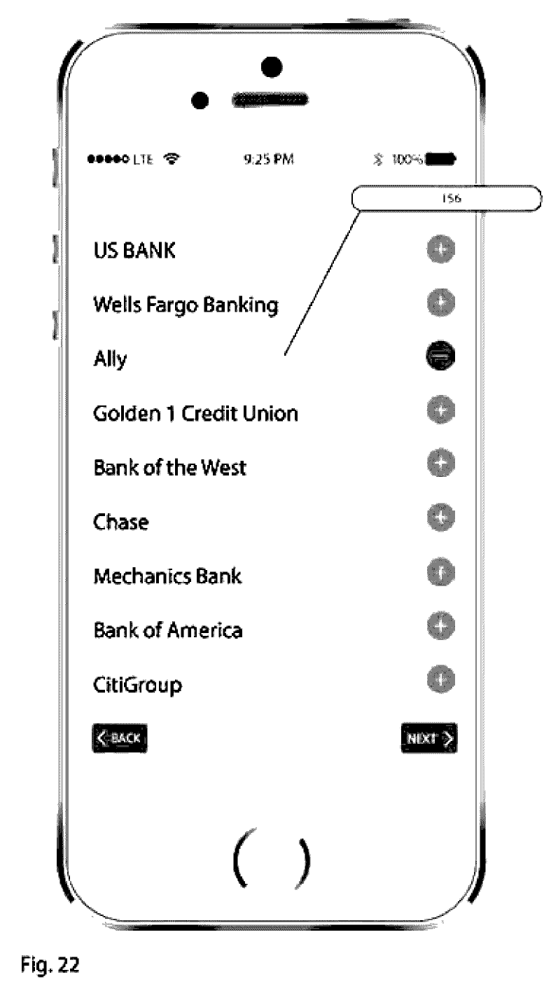

FIG. 22 perspective view of App selecting financial institution screen displaying item 156, this screen offers the wearer financial services and aid to ease the stress during pregnancy encouraging a healthier delivery and less stress;
Item—156—Selecting Financial Provider

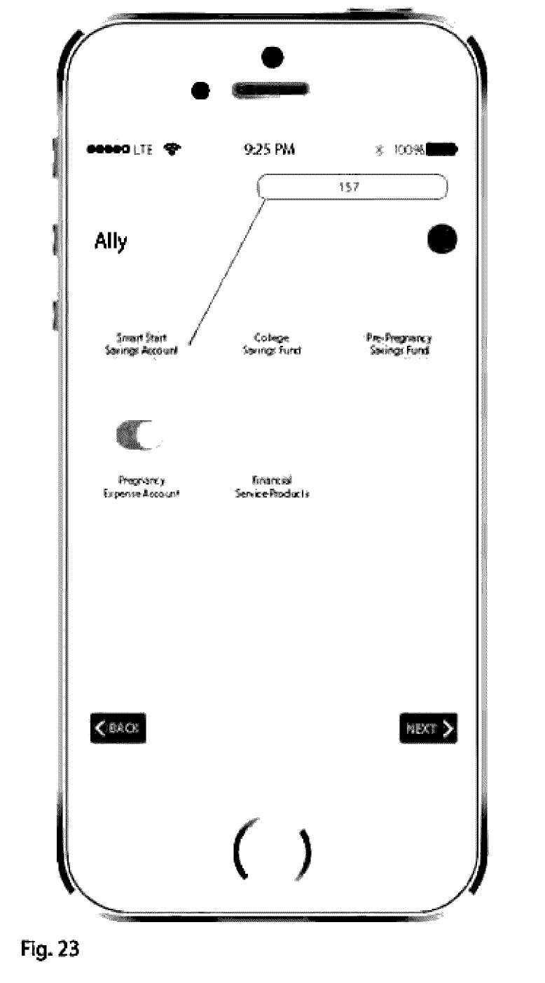

FIG. 23 perspective view of App selecting financial institution account screen continued displaying item 157;
Item—157—Home Security System

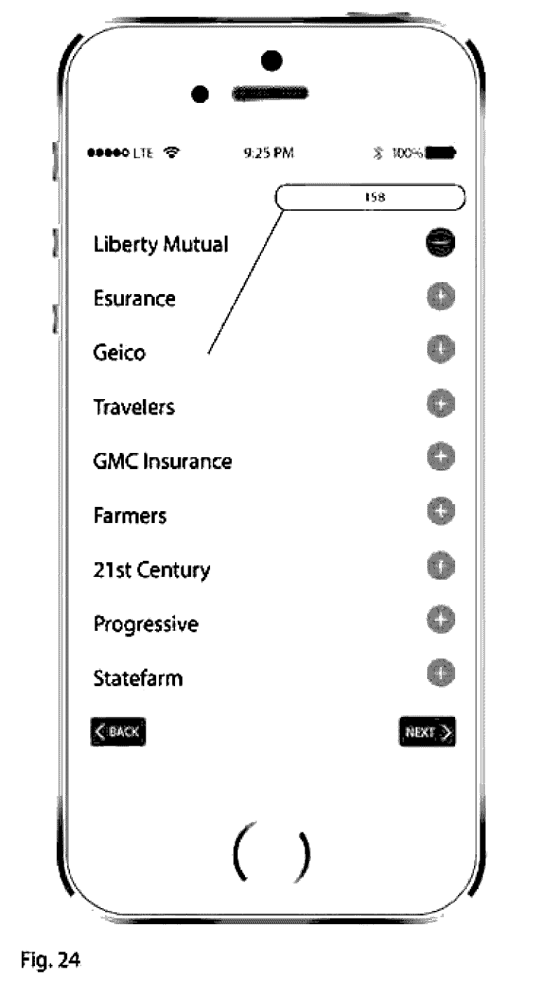

FIG. 24 perspective view of App selecting Insurance institution screen displaying item 158, this screen will prompt the wearer to choose insurance that will cover her and her family and unborn further relieving stress to encourage a healthier delivery;
Item—158—Choosing an insurance company

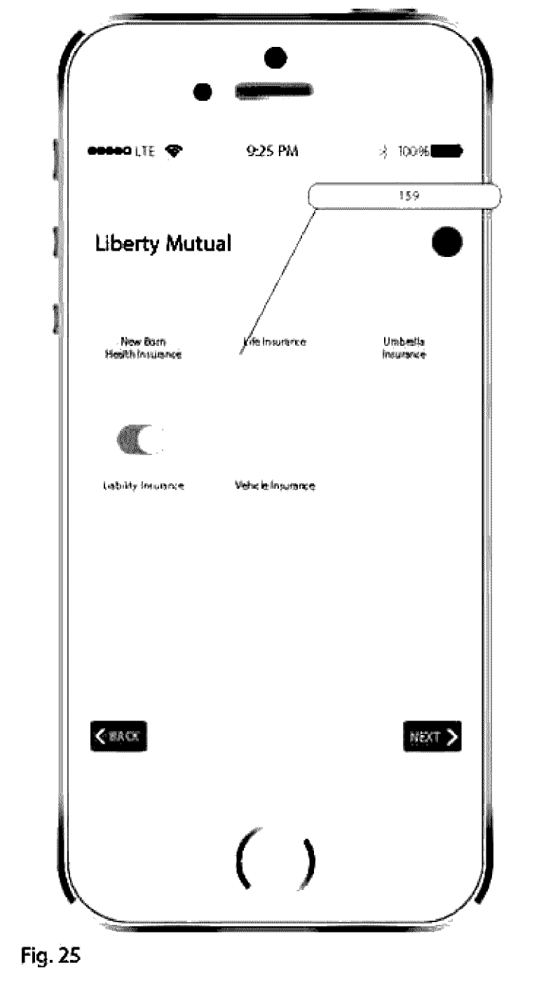

FIG. 25 perspective view of App selecting Insurance institution options screen continued displaying item 159;
Item—159—Choosing an insurance company continued

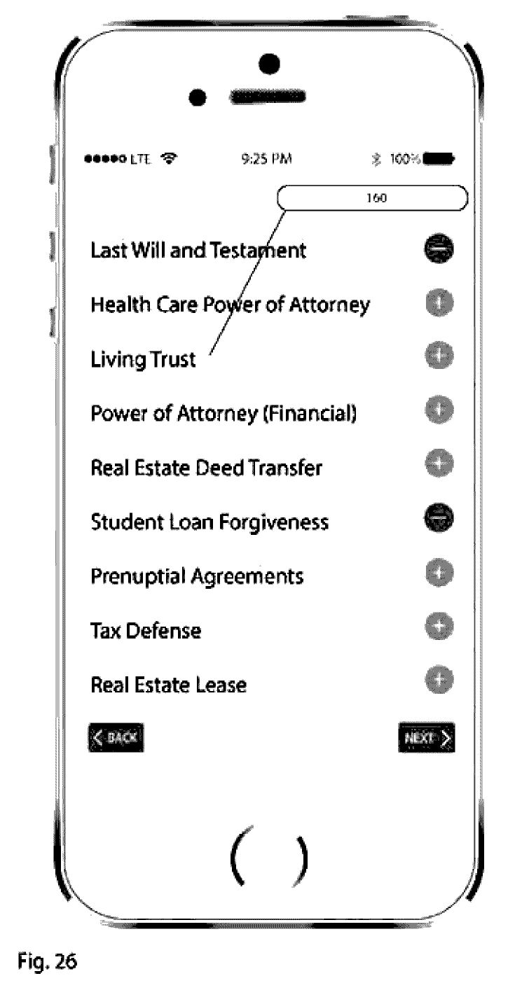

FIG. 26 perspective view of App selecting Legal protection plan screen displaying item 160, this screen is focused on protecting and planning the wearer for the future family relieving even more stress encouraging a healthier delivery;
Item—160—Choosing a legal plan through various legal institutes

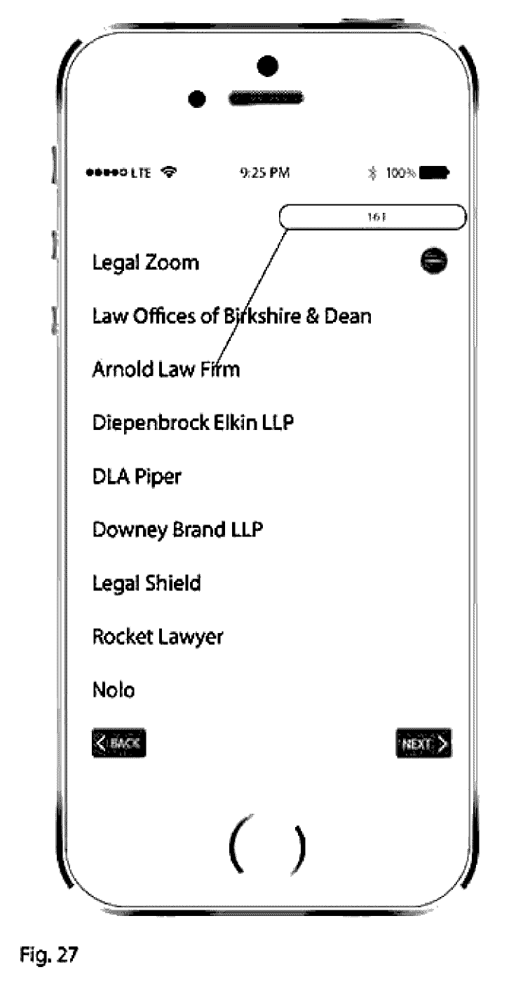

FIG. 27 perspective view of App selecting Legal firm screen continued displaying item 161;
Item—161—Choose a legal plan through various legal institutes continued

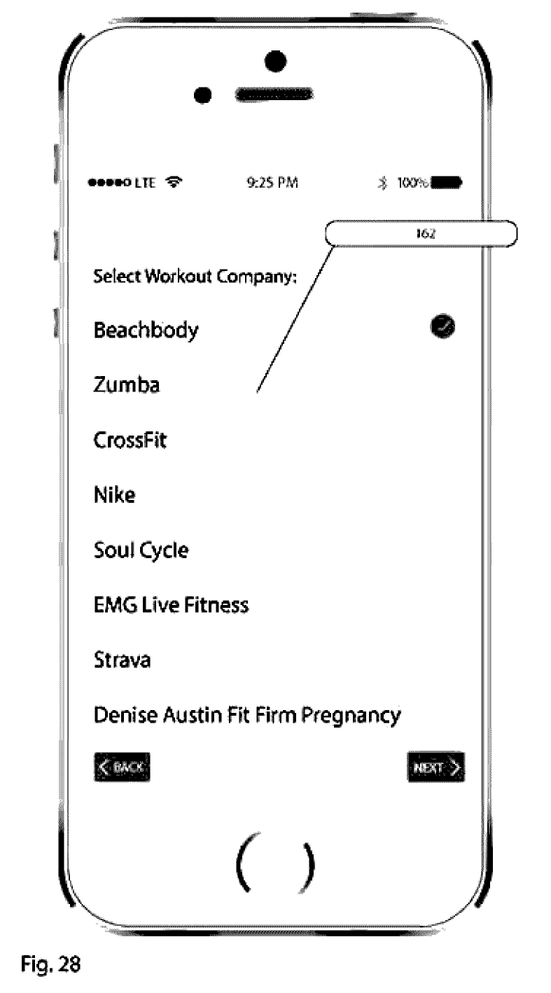

FIG. 28 perspective view of App selecting a Fitness company screen displaying item 162, this screen is important, it allows the wearer to choose different fitness routines or pregnancy safe stretches further encouraging a healthy delivery;
Item—162—Choose a fitness company to exercise

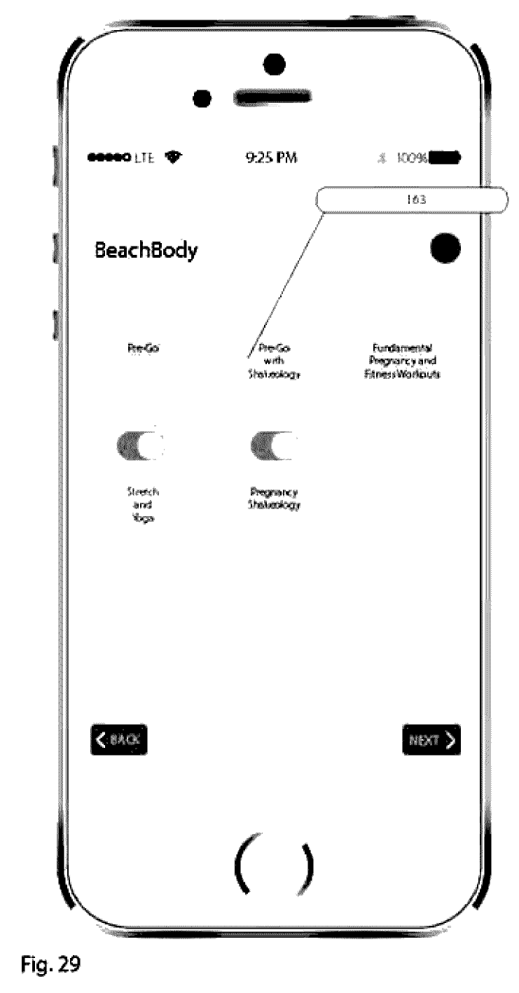

FIG. 29 perspective view of App selecting options for Fitness company screen continued displaying item 163;
Item—163—Choose a fitness company to exercise

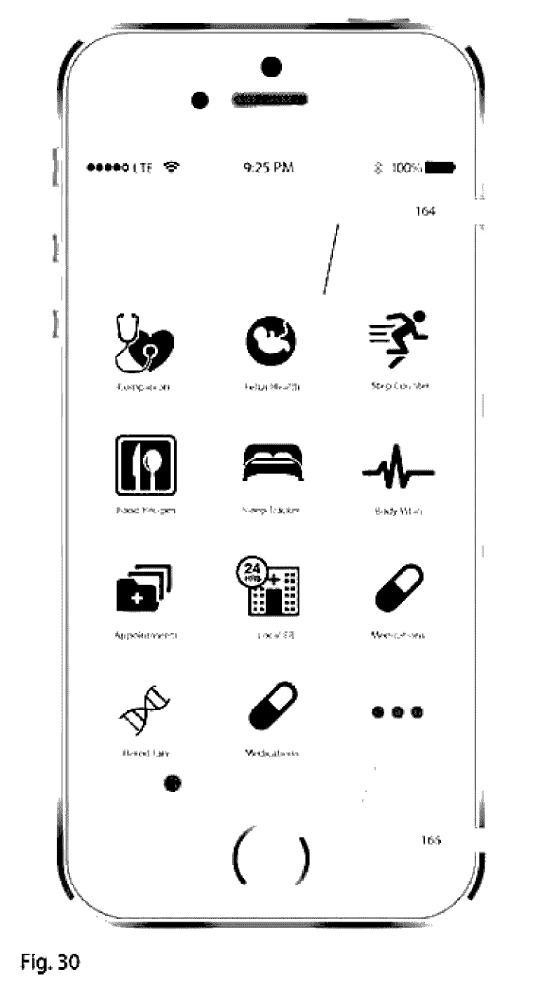

FIG. 30 perspective view of App Main screen displaying item 164-165, this screen can be expanded out to 1,000,000 different features and apps based on technology and Ai software;
Item—164—Main screen and root hierarchy which can be scaled with new app features
Item—165—Main screen and root hierarchy add new feature

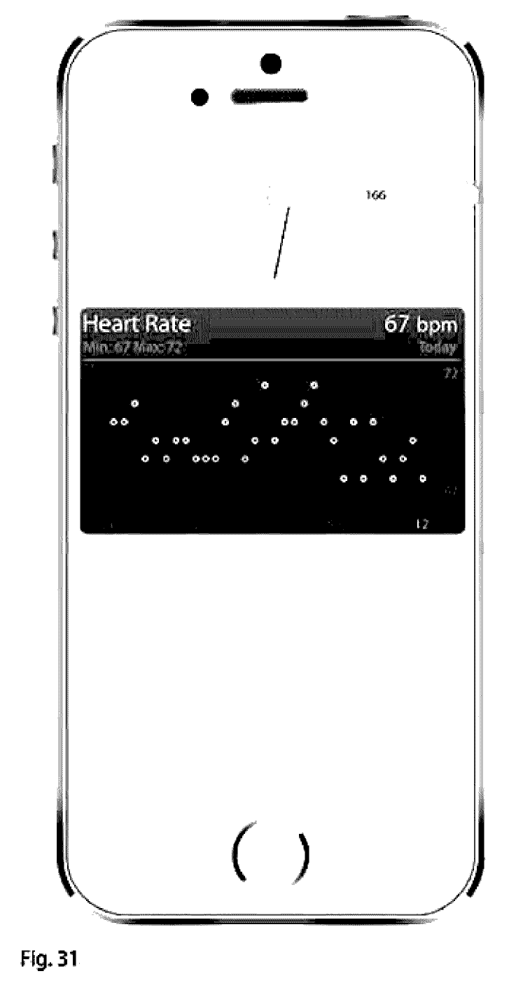

FIG. 31 perspective view of App checking heart rate screen displaying item 166, on this screen you can monitor your heart rate while working out to ensure you're hitting the right targets to make more progress;
Item—166—Main screen and root hierarchy which can be scaled with new app features

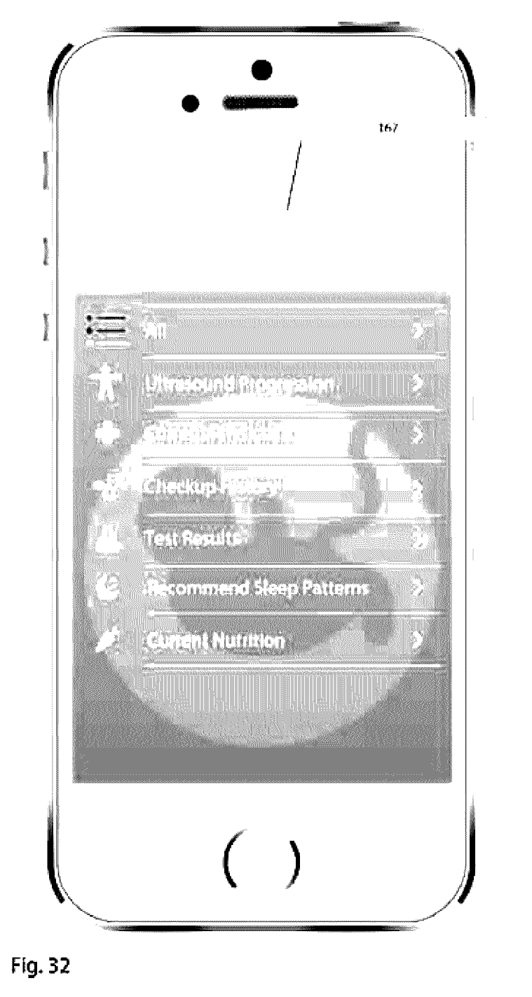

FIG. 32 perspective view of App fetus health screen displaying item 167, this screen will keep track of the process of the fetus and development giving the wearer more awareness of the progress;
Item—167—Main screen for App fetus of health screen

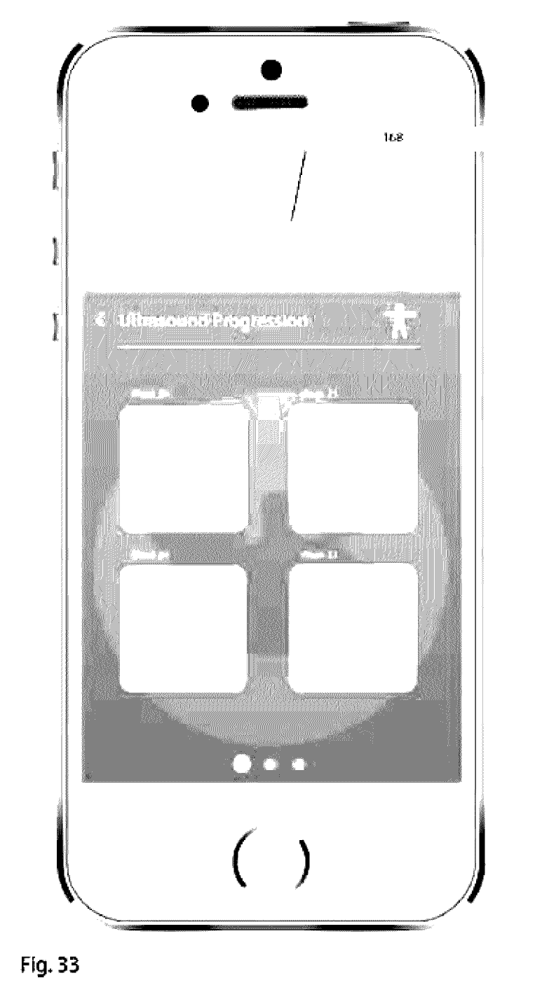

FIG. 33 perspective view of App fetus progress screen of the app continued displaying item 168;
Item—168—Main screen for App fetus of health screen continued

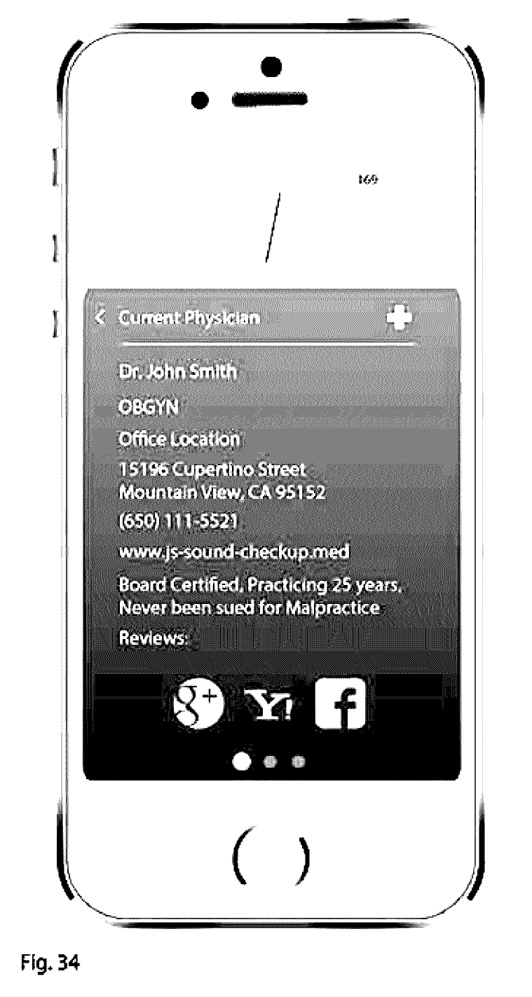

FIG. 34 perspective view of App checking currently selected doctor screen displaying item 169, this screen will have all of the medical doctors the wearer has setup and all appointment information with reminders;
Item—169—Main screen and root hierarchy which can be scaled with new app features

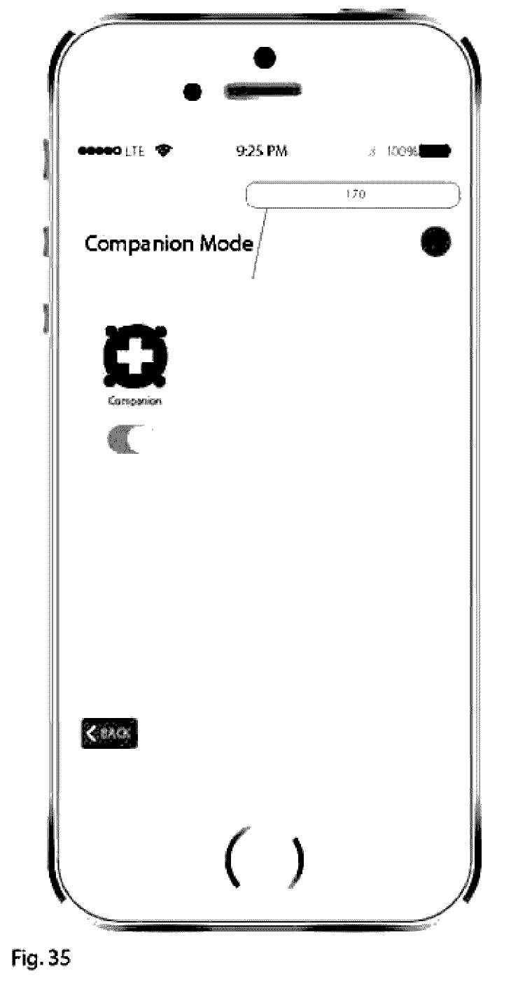

FIG. 35 perspective view of App selecting companion mode option screen displaying item 170, this is a feature that uses a proprietary algorithm that will use all components of the invention that creates suggestive technology and a cognitive interaction with the wearer from recommended exercise routines to foods to eat to medicines that are being taken for a specific hereditary disease;
Item—170—A feature of the app

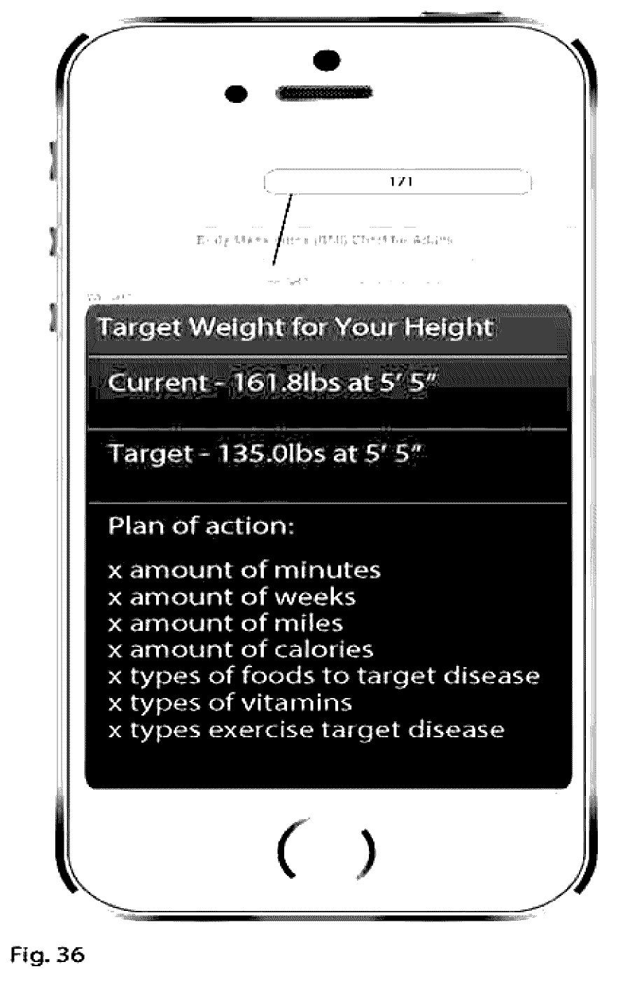

FIG. 36 perspective view of App body index screen displaying item 171;
Item—171—BMI feature of the app

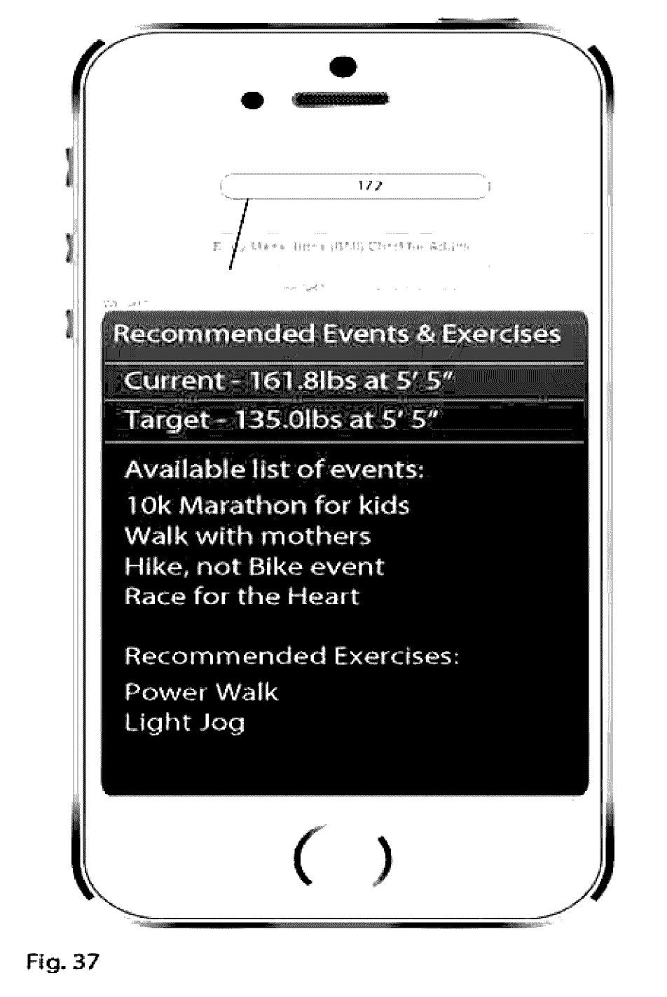

FIG. 37 perspective view of App selecting event options to stay active screen displaying item 172;
Item—172—A stay active feature of the app

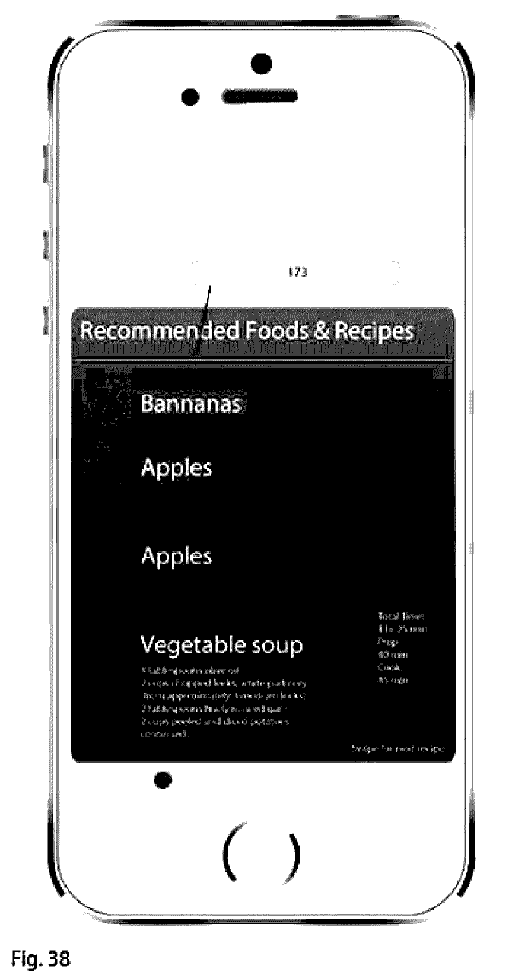

FIG. 38 perspective view of App recommended foods, veggies and healthy recipes screen displaying item 173;
Item—173—Main screen and root hierarchy which can be scaled with new app features

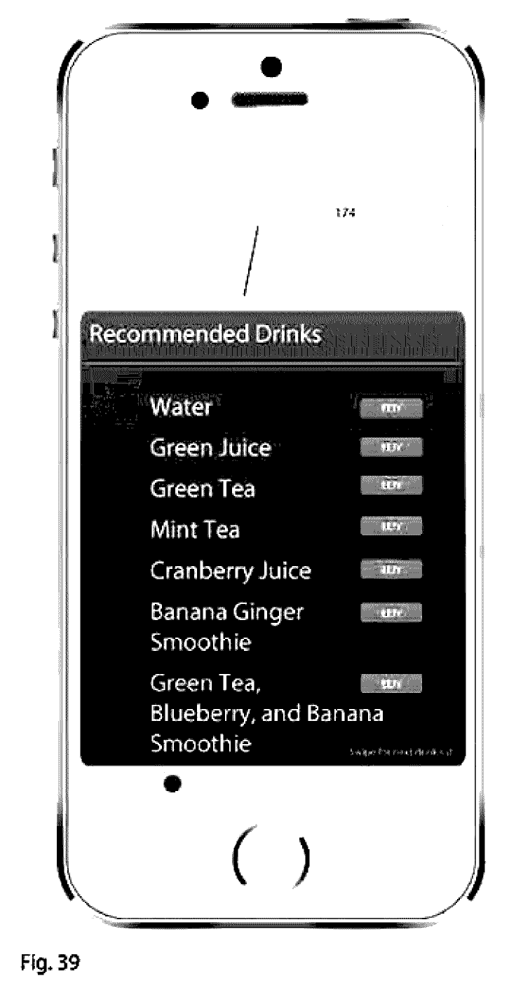

FIG. 39 perspective view of App recommended drinks screen displaying item 174;
Item—174—Buying option of different health drinks for the app

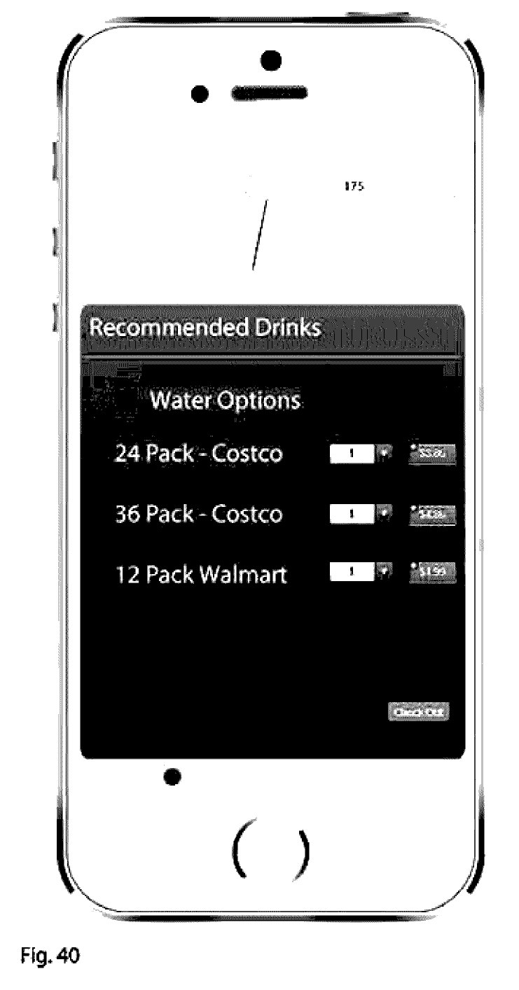

FIG. 40 perspective view of App recommended drinks screen continued displaying item 175;
Item—175—Buying options of different health drinks for the app

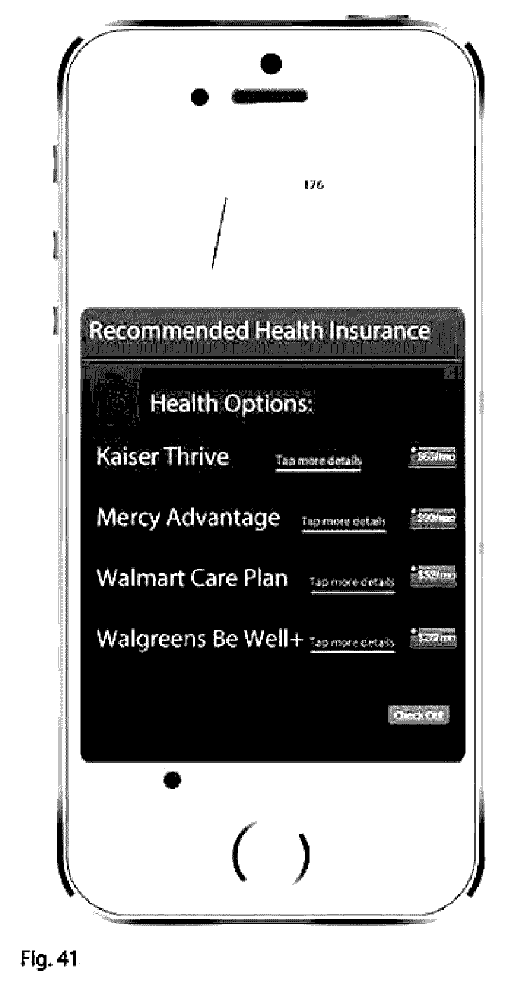

FIG. 41 perspective view of App Health Insurance plans screen displaying item 176;
Item—176—App offering insurance plans

FIG. 42 perspective view of App Pre-birth education download screen displaying item 177;
Item—177—App allows anyone to purchase pre-birth education

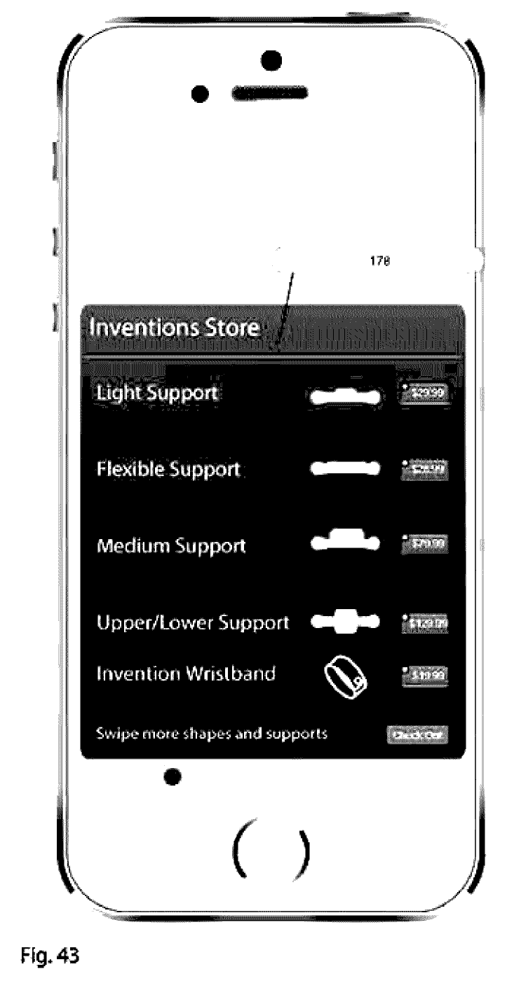

FIG. 43 perspective view of App Invention store screen displaying item 178;
Item—178—App allows for purchase of various shapes and material back support as part of the invention

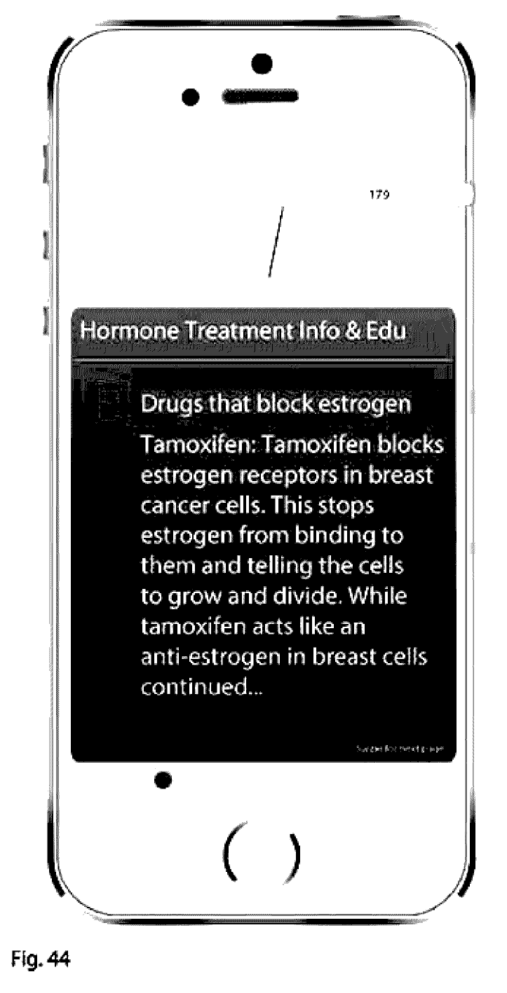

FIG. 44 perspective view of App Hormone treatment and information screen displaying item 179;
Item—179—App allows for hormone treatment programs and education information

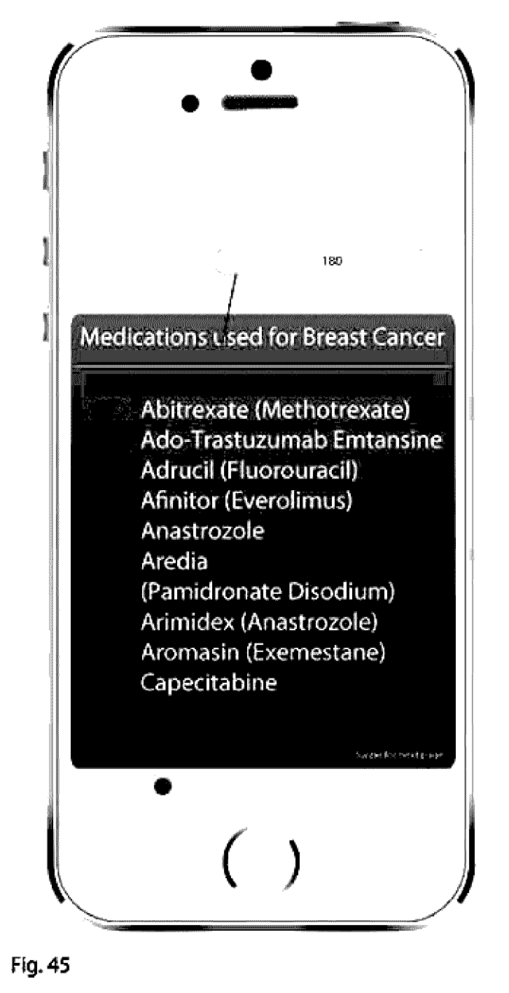

FIG. 45 perspective view of App Medications screen for diseases and cancers screen displaying item 180;
Item—180—Various medication options with approval of medical doctor

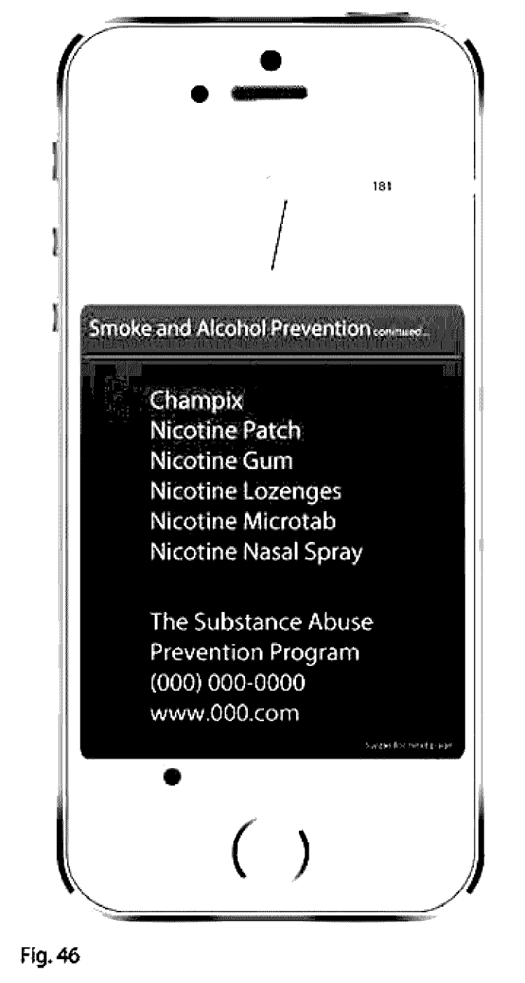

FIG. 46 perspective view of App Types of products and services to stop smoking/drinking alcohol screen displaying item 181;
Item—181—Education on services to help prevent or stop smoking and drinking alcohol

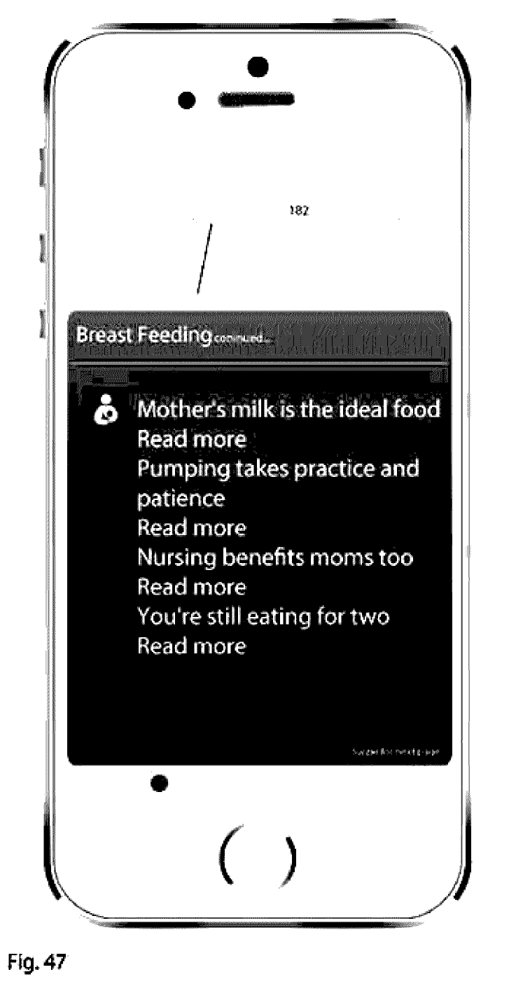

FIG. 47 perspective view of App Breast feeding education screen displaying item 182;
Item—182—Breast feeding education and information

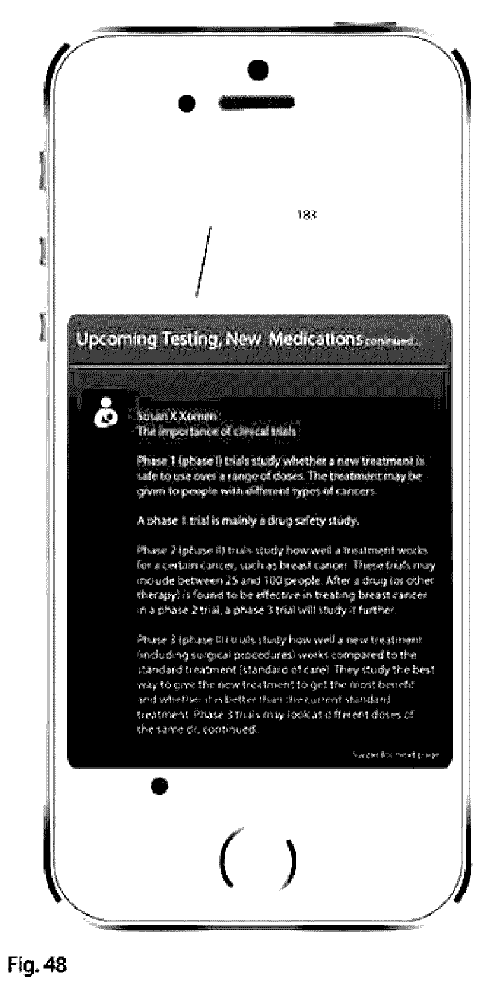

FIG. 48 perspective view of App Clinical trial information/testing/sign ups screen displaying item 183, clinical trials will further advance our understanding the disease and this screen will allow the wearer to participate in upcoming testing and new advance medicines to possible extend life and encourage hope;
Item—183—The app allows for various clinical trial information to further medicine

FIG. 49 perspective view of App Companion mode suggestion 1 screen displaying item 184, this is a feature that uses a proprietary algorithm that will use all components of the invention that creates suggestive technology and a cognitive interaction with the wearer. It will recommend foods to the wearer based on their selected hereditary disease;
Item—184—This is companion mode using suggestive technology as part of the invention

FIG. 50 perspective view of App Companion mode suggestion 2 screen continued displaying item 185;
Item—185—This is companion mode using suggestive technology as part of the invention

FIG. 51 perspective view of App Companion mode suggestion 3 screen continued displaying item 186;
Item—186—This is companion mode using suggestive technology as part of the invention

FIG. 52 perspective view of App Companion mode suggestion 4 screen continued displaying item 187;
Item—187—This is companion mode using suggestive technology as part of the invention

FIG. 53 perspective view of App Companion mode suggestion 5 screen continued displaying item 188;
Item—188—This is companion mode using suggestive technology as part of the invention

FIG. 54 perspective view of App Companion mode suggestion 6 screen continued displaying item 189;
Item—189—This is companion mode using suggestive technology as part of the invention

FIG. 55 perspective view of App Companion mode suggestion 7 screen continued displaying item 190;
Item—190—This is companion mode using suggestive technology as part of the invention

FIG. 56 perspective view of App Companion mode suggestion 8 screen continued displaying item 191;
Item—191—This is companion mode using suggestive technology as part of the invention

FIG. 57 perspective view of App Companion mode suggestion 9 screen continued displaying item 192;
Item—192—This is companion mode using suggestive technology as part of the invention

FIG. 58 perspective view of App Companion mode calling emergency services displaying items 193-203, this is a feature that uses a proprietary algorithm that will use all components of the invention that creates a connection with the wearer and medical response teams and hospitals where the invention transmit all data about the wearer on a secure connection so the emergency medical response team can proactively prepare to give the wearer a better chance at whatever the wearer is facing;
Item—193—Emergency app of companion mode alerting authorities and medical contacts inputted by wearer
Item—194—Companion mode contact hospital with wearers vitals and medical history
Item—195—Companion mode contact Ambulance/Helicopter
Item—196—Companion mode contact Ambulance/Helicopter
Item—197—Companion mode contacting 911
Item—198—Cloud transmitting data securely
Item—199—Cloud storage
Item—200—Cell towers transmitting data
Item—201—Medical data
Item—202—Invention cloud
Item—203—Invention Prenatal Belt

FIG. 59 perspective view of wearer using voice technology as part of the invention without a Smartphone displaying items 204-206, this allows the wearer to use Siri or S-voice to talk to the invention for different options;
Item—204—Microphone, Sound Decoder and Codec and Amplifier Chip
Item—205—Invention cloud which enables wearer to access food recipes, calories left to burn etc.
Item—206—Invention Prenatal Belt

FIG. 60 perspective view of wearer doing small blood test to transmit from home to medical facilities displaying items 207-214, this part of the invention is revolutionary for the health industry. It allows the wearer to transmit blood testing to local testing facilities to get immediate results and necessary actions from that facility;
Item—207—Blood pricking device
Item—208—Invention Prenatal Belt
Item—209—Cell tower
Item—210—Cloud Server and Proprietary Algorithm
Item—211—Cloud Platform Web Frontend and Backend
Item—212—Walgreens
Item—213—Walmart
Item—214—Local hospitals

FIG. 61 perspective view of App Companion mode suggestion 13 screen continued displaying items 215-223, this part of the invention will use fitness videos to help the wearer be more formed to maximize their work out and do correct movements. It also opens up a world of new interactive features;
Item—215—Class II Bluetooth Chip
Item—216—Invention Cloud
Item—217—Calorie Counter Board
Item—218—Gyroscope Board
Item—219—Detachable Layer with female connecting rod loop
Item—220—Types of material Options
Item—221—6-Axis Accelerometer Board
Item—222—Pedometer Board
Item—223—Detachable Layer with female connecting rod loop

FIG. 62 perspective view of App Companion mode suggestion 14 screen continued displaying items 224-226;
Item—224—Smartphone app
Item—225—Invention Prenatal Belt
Item—226—Small GPS Wrist Band

FIG. 63 perspective view of App Companion mode suggestion 15 screen continued displaying items 227-230;
Item—227—Smartphone app
Item—228—Invention Prenatal Belt
Item—229—Class II Bluetooth Chip
Item—230—Smart electric car connection

FIG. 64 perspective view of Smartphone app for industry related companies to use our API/Backend connection to sell their products and services such as fitness companies, banks, insurance, retailers, supplement companies, law firms, pre-birth education, cellular companies, independent retailers to sell different shapes and material for the interchangeable back support as part of the invention, personal instructors, pharmacies, medical offices, hospitals and any other related industry displaying items 231-233;
Item—231—App allowing any grocery store sell items through app so wearer can go into store and have all groceries ready in the bag and paid for that focuses on the health of the wearer through the Smartphone app and proprietary algorithm and programming, or any other related companies to sell their products based on what the wearer chooses
Item—232—Smartphone app hereditary disease selected by wearer and suggestive technology
Item—233—Smartphone app eCommerce store for wearer and retailers to interact with

FIG. 65 perspective view of smart battery management chip that will work in conjunction with battery cells that are installed into home from Tesla and other home power management companies for use, eliminating wasted and over used power generating companies, displaying items 234-268;
Item—234—Single Chip Smart Battery Charger Controller, 100% Compliant (Rev. 1.1) SM Bus Support Allows for Operation with or without Host, SM Bus Accelerator Improves SM Bus Timing, Wide Output Voltage Range
Item—235—Wireless battery charger from smart batter by Tesla
Item—236—Wireless charging and 2 way communication
Item—237—Patient Position Sensor
Item—238—Glucometer Sensor
Item—239—Body Temperature Sensor
Item—240—Blood Pressure Sensor (Sphygmomanometer) V2.0
Item—241—Pulse and Oxygen in Blood Sensor (SPO2)
Item—242—Airflow Sensor (Breathing)
Item—243—Galvanic Skin Response Sensor (GSR-Sweating)
Item—244—Electrocardiogram Sensor (ECG)
Item—245—Electromyography Sensor (EMG)
Item—246—Muscle Sensor v3
Item—247—Casing with male connecting rod loop
Item—248—Microcontroller completely assembled
Item—249—Stationary Layer
Item—250—Microcontroller Circuit Board with bios and firmware
Item—251—Qualcomm Neuromorphic Chip
Item—252—Memory Chip
Item—253—Class II Bluetooth Chip
Item—254—Microphone, Sound Decoder and Codec and Amplifier Chip
Item—255—Auto Length Accelerometer-Based Board
Item—256—Altimeter Board
Item—257—6-Axis Accelerometer Board
Item—258—Gyroscope Board
Item—259—Rechargeable Pure Aluminum-Ion Battery with Integrated Power-Saving Accelerometers Board
Item—260—Health Sensors
Item—261—Calorie Counter Board
Item—262—Pedometer Board
Item—263—Bluetooth® Smart Technology Software
Item—264—Detachable Bluetooth Speakers
Item—265—GPS Nano-Spider technology and accelerometer boards
Item—266—pCell Chip and Board
Item—267—Ai software
Item—268—Proprietary Algorithm

FIG. 66 perspective view of a woman using the invention to automatically tell the smart train where her destination is based on Smartphone app and automatically pay through Apple pay once acknowledged by wearer, displaying items 269-273;
Item—269—Smartphone app
Item—270—Invention Prenatal Belt
Item—271—Cell tower
Item—272—Smart train connection
Item—273—Smart train

FIG. 67 perspective view of a woman using the invention to automatically tell the Google self driving car her destination based on Smartphone app after acknowledged by the wearer, displaying items 274-278;
Item—274—Smartphone app
Item—275—Invention Prenatal Belt
Item—276—Cell tower
Item—277—Smart car connection
Item—278—Smart car

FIG. 68 perspective view of a woman using the invention to automatically tell the smart place where her destination is based on Smartphone app and automatically pay through Apple pay once acknowledged by wearer, displaying items 279-283;
Item—279—Smartphone app
Item—280—Invention Prenatal Belt
Item—281—Cell tower
Item—282—Smart plane connection
Item—283—Smart plane

FIG. 69 perspective view of a woman using the invention to automatically tell the smart helicopter where her destination is based on Smartphone app and automatically pay through Apple pay once acknowledged by wearer, displaying items 284-288;
Item—284—Smartphone app
Item—285—Invention Prenatal Belt
Item—286—Cell tower
Item—287—Smart helicopter connection
Item—288—Smart helicopter

FIG. 70 perspective view of a woman using the invention to automatically tell the smart train where her destination is based on Smartphone app and automatically pay through Apple pay once acknowledged by wearer, displaying items 289-293;
Item—289—Smartphone app
Item—290—Invention Prenatal Belt
Item—291—Cell tower
Item—292—Smart flying car connection
Item—293—Smart flying car

FIG. 71 perspective view of a U.S. soldier wearing the invention that connects to the cell tower, smart tank connection, smart tank, when heart rate, blood pressure goes up suddenly, the invention will signal the smart tank to go weapons active and reads the soldiers body so the soldier can react more efficiently in combat, displaying items 294-298;
Item—294—Smartphone app
Item—295—Invention Prenatal Belt
Item—296—Cell tower
Item—297—Smart tank connection
Item—298—Smart tank car
Item—299—Patient Position Sensor
Item—300—Glucometer Sensor
Item—301—Body Temperature Sensor
Item—302—Blood Pressure Sensor (Sphygmomanometer) V2.0
Item—303—Pulse and Oxygen in Blood Sensor (SPO2)
Item—304—Airflow Sensor (Breathing)
Item—305—Galvanic Skin Response Sensor (GSR-Sweating)
Item—306—Electrocardiogram Sensor (ECG)
Item—307—Electromyography Sensor (EMG)
Item—308—Muscle Sensor v3
Item—309—Casing with male connecting rod loop
Item—310—Microcontroller completely assembled
Item—311—Stationary Layer
Item—312—Microcontroller Circuit Board with bios and firmware
Item—313—Qualcomm Neuromorphic Chip
Item—314—Memory Chip
Item—315—Class II Bluetooth Chip
Item—316—Microphone, Sound Decoder and Codec and Amplifier Chip
Item—317—Auto Length Accelerometer-Based Board
Item—318—Altimeter Board
Item—319—6-Axis Accelerometer Board
Item—320—Gyroscope Board
Item—321—Rechargeable Pure Aluminum-Ion Battery with Integrated Power-Saving Accelerometers Board
Item—322—Health Sensors
Item—323—Bluetooth® Smart Technology Software
Item—324—GPS Nano-Spider technology and accelerometer boards
Item—325—pCell Chip and Board
Item—326—Ai software
Item—327—Proprietary Algorithm

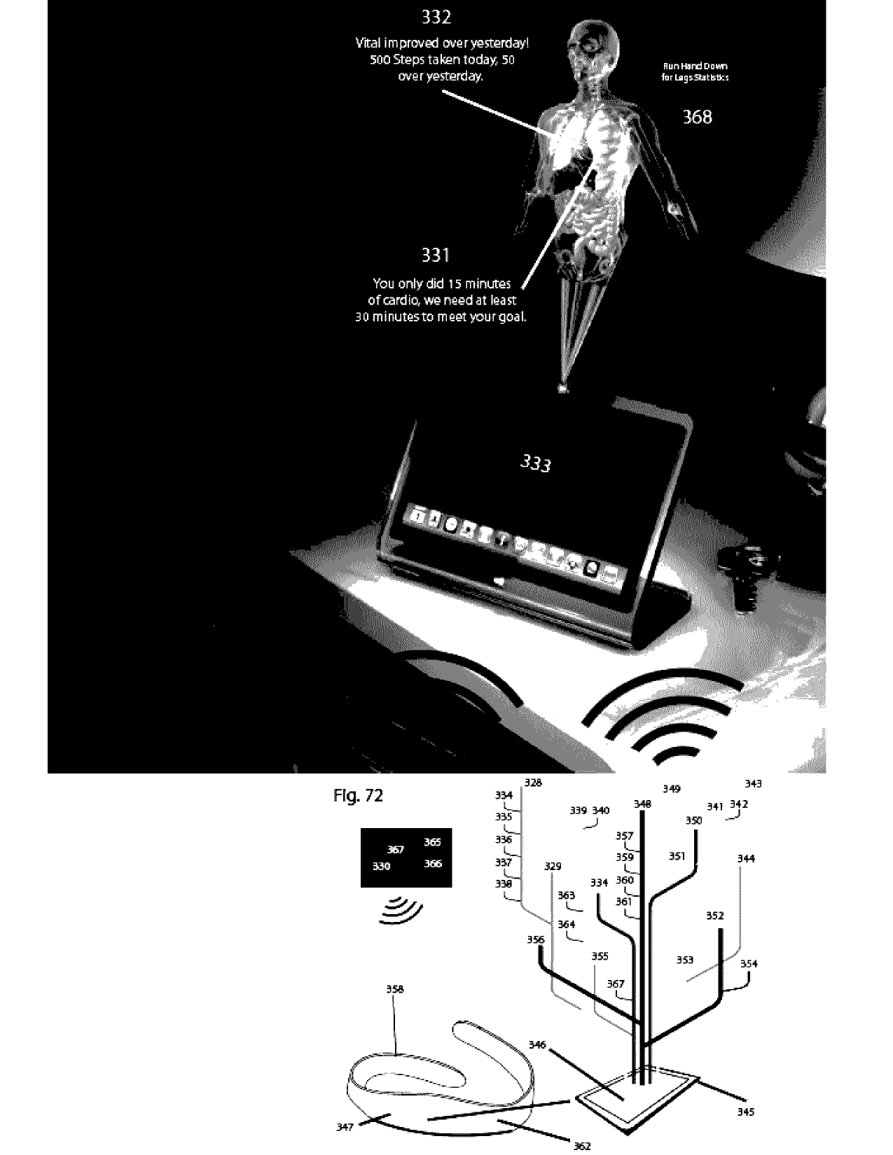

FIG. 72 perspective view of the invention parts using all components to project holographic information and allowing the wearer to interact with the invention using a Holographic Processing Unit (HPU), Wi-Di connection and Digital Signal Processor (DSP) features, displaying items 328-368:
Item—328—Wi-Di Chip Technology
Item—329—Holographic Processing Unit (HP U)
Item—330—Smartphone app
Item—331—Suggestive Technology
Item—332—Suggestive Technology
Item—333—User supplied iPad or other display screen
Item—334—Digital Signal Processor (DSP)
Item—335—Patient Position Sensor
Item—336—Glucometer Sensor
Item—337—Body Temperature Sensor
Item—338—Blood Pressure Sensor (Sphygmomanometer) V2.0
Item—339—Pulse and Oxygen in Blood Sensor (SPO2)
Item—340—Airflow Sensor (Breathing)
Item—341—Galvanic Skin Response Sensor (GSR-Sweating)
Item—342—Electrocardiogram Sensor (ECG)
Item—343—Electromyography Sensor (EMG)
Item—344—Muscle Sensor v3
Item—345—Casing with male connecting rod loop
Item—346—Microcontroller completely assembled
Item—347—Stationary Layer
Item—348—Microcontroller Circuit Board with bios and firmware
Item—349—Qualcomm Neuromorphic Chip
Item—350—Memory Chip
Item—351—Class II Bluetooth Chip
Item—352—Microphone, Sound Decoder and Codec and Amplifier Chip
Item—353—Auto Length Accelerometer-Based Board
Item—354—Altimeter Board
Item—355—6-Axis Accelerometer Board
Item—356—Gyroscope Board
Item—357—Rechargeable Pure Aluminum-Ion Battery with Integrated Power-Saving Accelerometers Board
Item—358—Health Sensors
Item—359—Calorie Counter Board
Item—360—Pedometer Board
Item—361—Bluetooth® Smart Technology Software
Item—362—Detachable Bluetooth Speakers
Item—363—GPS Nano-Spider technology and accelerometer boards
Item—364—pCell Chip and Board
Item—365—Ai software
Item—366—Proprietary Algorithm
Item—367—Ai Cloud software
Item—367—Hardware Ai software
Item—368—Hologram to next page of Smartphone App

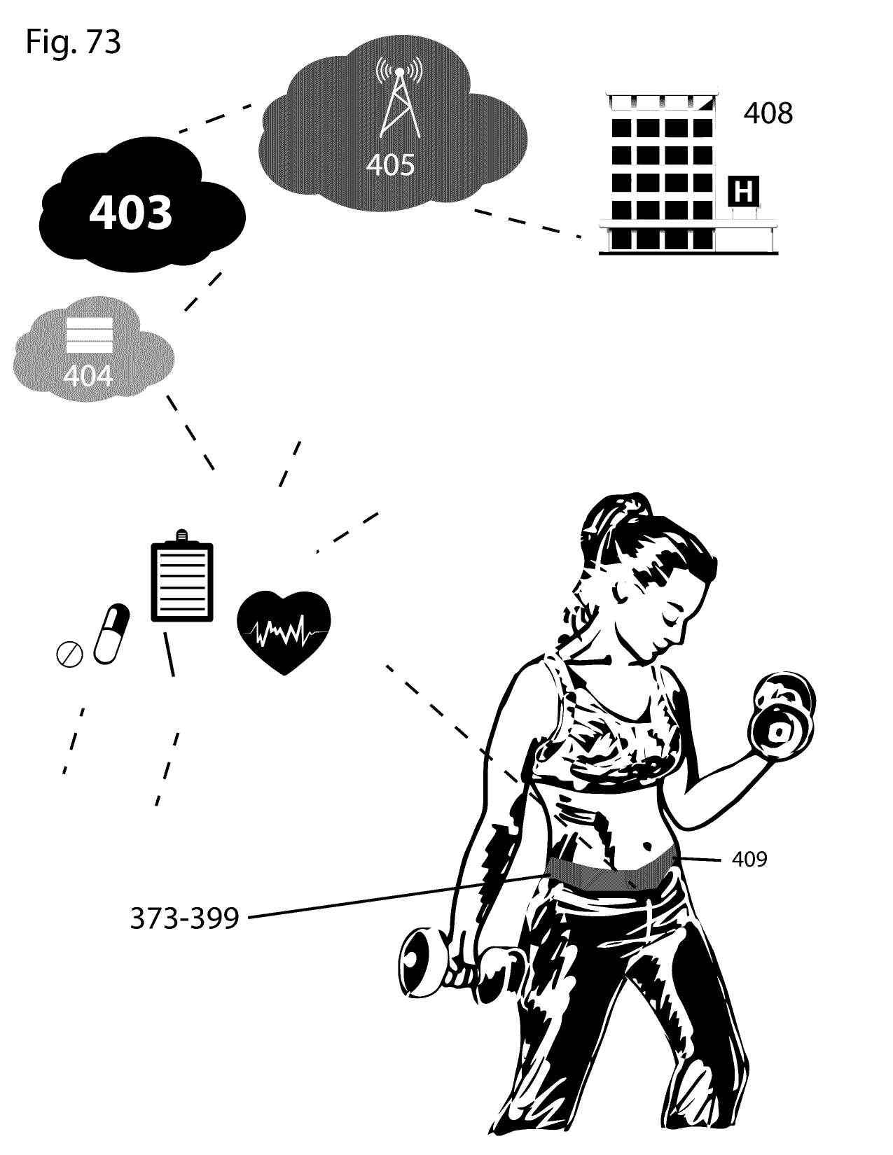

FIG. 73 perspective view of the invention being used to assess the patient for fatigue, hydration, stress, heart rate and other vitals for a regular health check, displaying item 369-409:
Item—369—Patient Position Sensor
Item—370—Glucometer Sensor
Item—371—Body Temperature Sensor
Item—372—Blood Pressure Sensor (Sphygmomanometer) V2.0
Item—373—Pulse and Oxygen in Blood Sensor (SPO2)
Item—374—Airflow Sensor (Breathing)
Item—375—Galvanic Skin Response Sensor (GSR-Sweating)
Item—376—Electrocardiogram Sensor (ECG)
Item—377—Electromyography Sensor (EMG)
Item—378—Muscle Sensor v3
Item—379—Casing with male connecting rod loop
Item—380—Microcontroller completely assembled
Item—381—Stationary Layer
Item—382—Microcontroller Circuit Board with bios and firmware
Item—383—Qualcomm Neuromorphic Chip
Item—384—Memory Chip
Item—385—Class II Bluetooth Chip
Item—386—Microphone, Sound Decoder and Codec and Amplifier Chip
Item—387—Auto Length Accelerometer-Based Board
Item—388—Altimeter Board
Item—389—6-Axis Accelerometer Board
Item—390—Gyroscope Board
Item—391—Rechargeable Pure Aluminum-Ion Battery with Integrated Power-Saving Accelerometers Board
Item—392—Health Sensors
Item—393—Calorie Counter Board
Item—394—Pedometer Board
Item—395—Bluetooth® Smart Technology Software
Item—396—GPS Nano-Spider technology and accelerometer boards
Item—397—pCell Chip and Board
Item—398—Nano-Sensing Integrated Circuit
Item—399—Hardware Ai software
Item—400—Ai software
Item—401—Proprietary Algorithm
Item—402—Ai Cloud software
Item—403—Cloud transmitting data securely
Item—404—Cloud storage
Item—405—Cell towers transmitting data
Item—406—Medical data
Item—407—Cloud
Item—408—Medical facility evaluating health from remote facility
Item—409—Invention Prenatal Belt

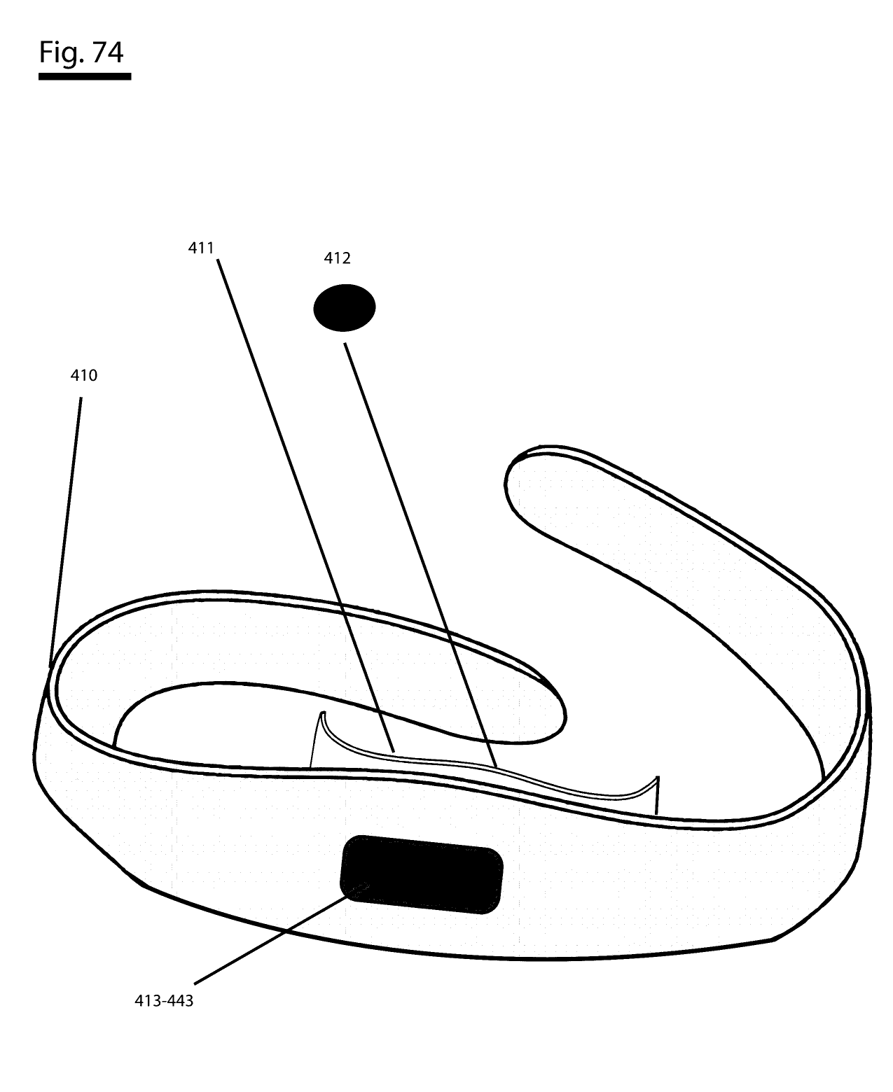

FIG. 74 perspective view of detachable shape that can include any various sensors to fit each unique wearer depending on their health condition and able to transmit to any medical facility using the invention, displaying items 410-443;
Item—410—Stationary Layer
Item—411—Detachable Layer with female connecting rod loop
Item—412—Various types of sensors and shapes embedded into them
Item—413—Patient Position Sensor
Item—414—Glucometer Sensor
Item—415—Body Temperature Sensor
Item—416—Blood Pressure Sensor (Sphygmomanometer) V2.0
Item—417—Pulse and Oxygen in Blood Sensor (SPO2)
Item—418—Airflow Sensor (Breathing)
Item—419—Galvanic Skin Response Sensor (GSR-Sweating)
Item—420—Electrocardiogram Sensor (ECG)
Item—421—Electromyography Sensor (EMG)
Item—422—Muscle Sensor v3
Item—423—Casing with male connecting rod loop
Item—424—Microcontroller completely assembled
Item—425—Stationary Layer
Item—426—Microcontroller Circuit Board with bios and firmware
Item—427—Qualcomm Neuromorphic Chip
Item—428—Memory Chip
Item—429—Class II Bluetooth Chip
Item—430—Microphone, Sound Decoder and Codec and Amplifier Chip
Item—431—Auto Length Accelerometer-Based Board
Item—432—Altimeter Board
Item—433—6-Axis Accelerometer Board
Item—434—Gyroscope Board
Item—435—Rechargeable Pure Aluminum-Ion Battery with Integrated Power-Saving Accelerometers Board
Item—436—Health Sensors
Item—437—Calorie Counter Board
Item—438—Pedometer Board
Item—439—Bluetooth® Smart Technology Software
Item—440—GPS Nano-Spider technology and accelerometer boards
Item—441—pCell Chip and Board
Item—442—Nano-Sensing Integrated Circuit
Item—443—Hardware Ai software

### CONTINUED DESCRIPTION OF INVENTION

Under the model that an expectant mother is using the invention, the functionality will be explained in that fashion:

She would turn on the invention FIG. 1 and FIG. 2 and all items, allowing the invention to cycle on. She would then use the invention FIG. 1 and FIG. 2 and all items and place it laterally on her waist and adjust to her comfort.

She can then proceed with using the invention as all features and functionalities of the invention FIG. 1 and FIG. 2 and all items 1-366 are in ready status; such as the Bluetooth speakers; pedometer and calorie counter, sleep trackers, heart rate trackers and other medical monitoring functionalities.

She would then launch the inventions FIG. 1 and FIG. 2 and all items free Smartphone app FIG. 2 item 27 through any internet connected device such as an iPhone, Android, Tablet, PC, Smart TV etc.

She would then setup her new account through the invention item 27, FIG. 16-28 She would proceed to answer all questions and choose options of the invention item 27, FIG. 16-28

### Screen Interface 1

The first screen will be white in color; through computer programming language, flash animation and/or gif slides or other animation software file output, the Smartphone app FIG. 2 item 27 will fade in the logo and play a melody. After a few seconds the logo will fade out. The invention item 27 will then bring up the second screen.

### Purpose

Introduce the expectant mother to the invention item 27 and allowing the expectant mother to know which Smartphone app FIG. 2 item 27 she launched, brand identity awareness and sets the tone of the Smartphone app FIG. 2 item 27. SEE FIG. 15, Functionality of Screen Interface 1

### 1 Start Up Screen, 2 Logo, Screen Interface 2

### Screen Interface 2

The second screen will be white in color; this screen will display a number of fields. These fields range from, but not limited to, the first and last name of the expectant mother, height and weight BMI, address, phone number, email address, credit card information through Apple Pay to your current health provider and other personal identifying information. After keying in all of your personal information, the invention item 27 will then bring up the third screen.

### Purpose

After the expectant mother inputs all pertinent information into the invention item 27, the invention item 27 will then calculate the information through programming language, mathematical computation and syntax combinations for control structures to enable displayed information such as body mass index charts, it will detect medical offices and services, set, change or cancel medical office appointments, health insurance options for the expectant mother and fetus, it will display hospitals and other organizations that connect through the Smartphone app FIG. 2 item 27. SEE FIG. 16, Functionality of Screen Interface 2

1 Name, Address, Phone, Email, Credit Card for purchases and other information 2 Registration Screen.

### Screen Interface 3

The third screen will be white in color; it will ask the expectant mother to check each box of hereditary diseases that is prevalent in the family history that causes health problems. The invention item 27 will then bring up the fourth screen.

### Purpose

This screen interface has a two part purpose; the first purpose would be to enable the expectant mother to select different diseases. The invention item 27 will then calculate the information through programming language, mathematical computation and syntax command combinations for control structures to enable the invention item 27 to display the data on different screens throughout the invention item 27.

In doing so, the invention item 27 along with the items 1 and FIG. 2 and all items 1-105, item 30 will provide a heuristic health solutions platform that the expectant mother can follow. The invention item 27 will also provide medically certified information written by medical professionals on the diseases causes, symptoms, treatments, home care instructions, seek immediate attention if necessary and make sure you list for educational purposes about the hereditary disease.

The second purpose would be for the invention item 27 interface to display meal plans, exercise routines and other health benefit information that the expectant mother would follow to aim at the diseases. Please see the continuation of the second purpose below listed as example.

### EXAMPLE

If an end-user, expectant mother in this example, has early signs of acid reflux disease, also referred to as Gastroesophageal Reflux Disease GERD, or if it runs in the family genes and the expectant mother doesn't know how to identify the symptoms. The invention, the Smartphone app FIG. 2 item 27 will educate the expectant mother on the symptoms, i.e. burning in lower part of mid-chest, severe heart-burn, worsening pain at rest, post-meal pain, bitter taste, hoarseness, trouble swallowing, excess mucus buildup and constant clearing of throat.

The invention item 27 will display acid inducing foods to stay away from like red sauces, bacon fat, lard, high-fat desserts or snacks like ice cream and potato chips, cream sauces, gravies, and creamy salad dressings, spicy seasonings and other foods that can elicit irritation and accelerate the GERD disease. By this very acceleration, it may cause scarring in the esophagus, which may lead to cancer if untreated or uneducated about it.

The invention item 27 will display acid reducing foods and recipes that help the expectant mother stay healthy and slow and/or prevent the GERD disease, like oatmeal, ginger, Aloe Vera, salad and other healthy foods. As the expectant mother is consuming these acid reducing foods, she is scenting the amniotic fluid and introducing the fetus to the acid reducing food culture so that there's a physiologically transfer to the fetus so the fetus has an advantage over the disease before the fetus is born and can possibly extend the future of the unborn child while ensuring a healthy development. This is possible as the fetus has food memory and after birth will prefer the acid reducing foods over acid inducing foods. Studies show that what a woman eats during pregnancy not only nourishes her baby in the womb, but may shape food preferences later in life through the fetus's palate and food memories.

### See FIG. 17

Functionality of Screen Interface 3 1 Selecting Hereditary Disease, 2 Pre-determines what foods and exercise will suppress and/or prevent the selected diseases.

### Screen Interface 4

The fourth screen will be white in color; this screen will assist the expectant mother with seamlessly managing her pregnancy from the medical side. The invention item 27 will then bring up the fifth screen.

### Purpose

This will enable the expectant mother to be fully connected to vital appointment times, dates and receive medical notifications. This screen will also assist the expectant mother in connecting to medical doctors, most visited medical offices, local hospitals, current appointment information, medications currently taking, OBGYN checkup recommended times based on pregnancy timeline, medical alerts, messages and even a live chat line with certified medical professionals to ask general questions about pregnancy, what to expect, healthy choices and hundreds of other health benefiting information, SEE FIG. 18-21, Functionality of Screen Interface 4, 1 the invention item 27 setup 2 You search and select your doctor, medical office and reminders.

### Screen Interface 5

The fifth screen will be white in color; this screen will help in planning the financial future for the unborn child and also the budgeting of the pregnancy of the expectant mother. The invention item 27 will then bring up the sixth screen.

### Purpose

To provide the expectant mother with peace of mind that she is preparing her unborn child for the future financially. The invention item 27 will offer financial planning, life insurance options, college saving funds, medical expense accounts, how to save information, options to open special bank accounts for the expenses of the pregnancy and early years, new family protection and setup options, and other various features that give the expectant mother options that will help alleviate her stress levels and focus on a healthy development of the child. SEE FIG. 22-27, Functionality of Screen Interface 5

1 Choose a financial institution, 2 you setup your savings accounts for your unborn child's future, insurance and other valuable financial information.

### Screen Interface 6

The sixth screen will be white in color; this screen will enable expectant mothers to sign up with exercise programs during the expectant mothers pregnancy and accounts even after the pregnancy. The invention item 27 will then bring up the seventh screen.

### Purpose

This will allow the expectant mother to access hundreds of work out videos, access exclusive pregnancy safe workouts, and give options for special protein powders that is medically proven to help develop a healthy fetus while still in the womb and other products to build a healthy lifestyle for the expectant mother and fetus. SEE FIG. 28

Functionality of Screen Interface 6 1 Choose a fitness provider listed.

### Screen Interface 7

The seventh screen will be white in color; this is the main screen which allows the expectant mother to have unlimited access to all benefits of the invention.

### Purpose

The main screen will display settings, options, icons and health data i.e. target healthy weight, calories burned, steps taken, miles walked, heart rate, blood pressure and other vital information to keep the mother in tune with her body and fetus.

The main screen will also display basics and most frequent pages used i.e. meal plans for certain diseases, money management, exercises and workout routines. The main screen can also toggle into options where the wearer can select options such as companion mode.

Different features of the invention item 27 which uses item 2, item 11, item 12, item 13, item 16, item 17, item 18, item 19, item 21, item 22, item 23, item 26 and item 30 in FIG. 1. and FIG. 2, give life to a new way of interacting with technology and health.

The feature when enabled, will operate under the selected wearer's hereditary disease and through proprietary algorithm using mathematical computation, computer science, programming language, special software and new CPU technology which is readily available and modeled to use a neural processing which can compute sensory data and tasks modeled on biological brains. This will allow the invention to offer suggestive alerts to the wearer through Ai software in which Google has demonstrated in past presentations.

The purpose of companion mode, SEE FIG. 35, it offers suggestive actions that target the selected hereditary diseases in the human body to prevent and slow progresses before it succeeds. The invention item 27 which uses item 2, item 11, item 12, item 13, item 16, item 17, item 18, item 19, item 21, item 22, item 23, item 26 and item 30 in FIG. 1. and FIG. 2 is programmed and connects with databases online that have every bit of data on diseases, causes, treatments etc. and its relation to the effects on the human body and what organs it targets.

The invention using item 27 which uses item 2, item 11, item 12, item 13, item 16, item 17, item 18, item 19, item 21, item 22, item 23, item 26 and item 30 in FIG. 1. and FIG. 2 will then suggest specific actions from the wearer to target the cancer through the body's natural capability. The following will be suggested, but not limited to:

Use the wearers registered information, SEE FIG. 16, to calculate how many calories, miles, types of food, vitamins and other health information to target a healthy physical status. The invention will then encourage the wearer through suggestive reasoning to hit the target weight, SEE FIG. 36.

Studies have proven that women who weigh over 175 pounds, the chances of breast cancer are about 25 percent higher than someone who weighs 132 pounds.

The invention will then set specific exercise routines that strengthens the body's ability to fight off the cancer if it starts to progress in the body. Studies have shown moderate daily exercises reduces breast cancer risk by 15 to 25 percent, SEE FIG. 37.

The invention will then calculate what foods to eat and even provide recipes to the wearer that deliver nutrients to the body to fight the cancer, SEE FIG. 38.

Studies have shown a healthy diet can lower the risk of breast cancer, and an unhealthy diet can increase your risk. Eating lots of fruits and vegetables, whole grains, and lean proteins is recommended. It's also recommended to use vegetable oils instead of animal fats, and avoid sugared drinks, refined carbohydrates, and fatty foods. Certain foods offer additional protection, including whole soy, especially if it's fermented. Soy has estrogens that attach to cells, but they don't have the same effect as body estrogen. Instead, the natural estrogens found in soy can block body estrogen from affecting cells. Other foods that can help protect against cancer include turmeric, garlic, olive oil, green tea, and seaweed

The invention will provide a list of drinks that are healthy. It will also have the option of entering in a drink and it will tell you if it's healthy or unhealthy, FIG. 39. Furthermore, the invention will have the option to make buying simple. The wearer can simply tap the number of items they wish to purchase, then tap the checkout button and the items will be shipped to the wearer's home address with registered information into the Smartphone app FIG. 2 item 27, SEE FIG. 16. This makes staying healthy easier. The invention further offers options to buy accessories, pre-birth educational music, health insurance, downloads and other items, SEE FIG. 40-43.

National Institutes of Health listed the consumption of alcoholic beverages as a “known human carcinogen” for the first time. Doctors advise avoiding alcohol entirely or greatly limiting drinking.

The invention will give alerts and educational information on hormone treatment, SEE FIG. 44.

For decades, women have used hormone therapy during and after menopause to combat symptoms such as hot flashes, irritability, and trouble sleeping. But recent research has shown that hormone replacement therapy may increase the risk of breast cancer.

The invention will provide a list of medications that have been used in breast cancer patients, SEE FIG. 45.

Women who are over age 60 or who have a family history of breast cancer, medical doctors advise that patients should consider taking an estrogen-blocking drug such as tamoxifen or raloxifene. Both drugs stop breast cells from being affected by estrogen.

According to the American Cancer Society, studies have shown that tamoxifen reduces the risk of breast cancer by about 50 percent and raloxifene reduces the risk by about 38 percent in women at higher risk for the disease. Because there are also risks associated with these drugs, medical doctors urge women to talk to their doctors about the pros and cons.

The invention will provide a list of references and educational classes for smoking and alcohol classes and most common ways to quit, SEE FIG. 46.

Although smoking is a factor in lung and other cancers, its role in breast cancer has been unclear. But a recent review by a Canadian panel of experts showed that both active smoking and exposure to secondhand smoke increase breast cancer risk in premenopausal women. The panel cited evidence from studies suggesting that women who start smoking at a young age are 20 percent more likely to develop breast cancer, and smoking for many years increases risk by up to 30 percent.

The invention will provide the wearer with education and information on breastfeeding initiation, duration, and exclusivity information, SEE FIG. 47.

Numerous studies have shown that women who breastfeed have a lower risk of breast cancer. The fact that women in developed countries tend to breastfeed for shorter periods or skip it completely is a major contributor to the high rates of breast cancer in these countries, according to an analysis of 47 studies in 30 countries.

When a woman is breastfeeding, medical doctors explain, their bodies produce higher levels of the hormone prolactin, which lowers the levels of estrogen. The longer a woman breastfeeds over a lifetime, the more protected she is against breast cancer, the analysis concluded. Medical doctors say that, if possible, women breastfeed for at least a year.

The invention will give alerts when new testing, trials, medications and other details of the cancer are available so the wearer can connect with the organizations, SEE FIG. 48.

Medical Doctors encourage women to consider participating in clinical trials studying ways to reduce the risk of breast cancer. For example, ongoing research is studying whether soy, vitamin D, green tea, and other supplements or foods affect breast cancer risk. By participating in a clinical trial, you have a chance to help all women, and you may end up lowering your own breast cancer risk at the same time.

The invention will continue to provide information on ways to target the many types of cancer and other diseases. The invention uses a proprietary algorithm that continuously recommends foods, drinks, recipes, remedies, exercise routines, environment suggestions, even levels of elevation that increase prevention, see reference 1, and other health impacting suggestive information to encourage and educate the wearer in an effort to move the wearer to a healthier lifestyle to prolong their life span.

Further capabilities of the invention are security features. The features will enable her to leave her house as she wishes and the security system automatically locks the doors and turns select lights on to deter criminals from breaking in. It also enables her to walk into different rooms that are preprogrammed with settings which will allow the lights to turn on/off automatically, and other features.

Another feature of the invention FIG. 1-FIG. 7, and all items is it's able to connect to her vehicle through item 11, item 12, item 14 and item 30. This feature allows her to walk up to her car and as she gets 10 feet from it, the vehicle starts, unlocks itself and the door opens using existing automaker technology via Bluetooth.

She can launch her BMI chart using the invention FIG. 1-FIG. 7, all items and see what a healthy weight would be for her height, and set the options using the calorie counter and step counter so she can start walking to meet the goals.

She can take a walk through the park and monitor her movements using the invention FIG. 1-FIG. 7, and all items to ensure she is burning the most calories, count her steps and other health information.

After she is done walking through the park and has to eat a small meal, the invention FIG. 1-FIG. 7, all items is also programmed with the hereditary disease GERD she selected. After she taps the button on the screen interface for recipes, it will recommend Butternut Squash Soup, and provide the recipe, i.e. 2 cups water, 2 lbs butternut squash, ½ tsp. salt, fresh ground black pepper, ½ tsp. dried, thyme leaves, ⅛ tsp., ground nutmeg and 1 cup water. This recipe has no specific GERD triggers and acid reducing, further suppressing the disease and possibly extending her life expectancy. She also selected Breast cancer as a hereditary cancer, so the invention will suggest healthy fruits and vegetables, exercise routines and other actionable recommendations to strengthen the body and target the cancer.

Two studies just released by the University of California, San Diego, and the Genesis Prevention Center at University Hospital in South Manchester, England, show that a meal plan rich in fruits and veggies and low in starchy carbohydrates may help prevent breast cancer and breast cancer recurrence

After she eats her mid-afternoon snack, she decides to lie down, she can launch the invention item 27 and check her sleep tracker to see how much sleep she needs or has left based on the tracker. Using the invention FIG. 1-FIG. 7, and all items will display, “a 30- to 60-minute nap during the day makes you more alert, sharpens memory, and generally reduces feelings of fatigue.” It will have a timer that will measure how long she sleeps and remaining sleep needed to hit her target.

After she takes her nap, she can then use the invention FIG. 1-FIG. 7, and all items to play instrumental classical music to her fetus. The invention FIG. 1-FIG. 7, and all items. The invention will display, “Researchers believe that musical training actually creates new pathways in the brain,” and have educational articles about playing music to your unborn child. Parents know that a quiet, gentle lullaby can soothe a fussy baby. And a majestic chorus can make us swell with excitement. In recent years, we've learned a lot about how the brain develops. Babies are born with billions of brain cells. Even pre-birth as a fetus, these cells are firing back and forth learning and developing the brain. During the first years of life, those brain cells form connections with other brain cells. Over time, the connections we use regularly become stronger. Children who grow up listening to music develop strong music-related connections. Some of these music pathways actually affect the way we think. Listening to classical music can improve our spatial reasoning, at least for a short time. And learning to play an instrument may have an even longer effect on certain thinking skills.

After she is done playing music for her fetus, she can use the invention FIG. 1-FIG. 7, and all items and check for upcoming appointments.

If she is feeling more pain in her back from the body change from the pregnancy she can re-adjuster the invention FIG. 1-FIG. 7, and all items to provide a firmer support and alleviate the pain. She even has the option of purchasing any size, material or composite with various densities such as foam and gel to offer greater back support.

As she sits in the living room talking to her friend on the phone while wearing the invention FIG. 1-FIG. 7, and all items, it transmits data and alerts her that her heart rate is too low and at an unhealthy rate while pregnant. The invention FIG. 1-FIG. 7, and all items will then display, “To support the expansion of your blood volume that occurs with pregnancy, your heart rate must rise to an additional 10 to 15 beats per minute. Generally, if you are pregnant and your heart rate is less than 60 beats per minute, your doctor should evaluate you thoroughly for any underlying cardiovascular or other medical issues.” This allows her to get up and take a walk or even walk around the home to raise her heart rate to a healthy level.

After the heart rate alert, she feels she would like to see a doctor for possible cardiovascular problems she may have after the experience. She can then use the invention FIG. 1-FIG. 7, and all items and tap on the main screen interface labeled doctor visits, and tap request. After she does this, her medical office will receive the request and send her appointment dates and times.

It's around dinner time, and she uses the invention FIG. 1-FIG. 7, and all items to find dinner recipes that are acid reducing. Then she walks to put her shoes on and walks out the door, the invention FIG. 1-FIG. 7, and all items detect she has left the home and automatically locks the doors and activates select lights. She walks down her driveway out to the street and the invention FIG. 1-FIG. 7, and all items detects she is 10 feet from the vehicle and it starts, the vehicle turns on and the door opens. She arrives at the store, walks away from her vehicle and the invention FIG. 1-FIG. 7, and all items shuts off the vehicle and locks the doors while enabling the alarm. As she walks through the store, she can use the invention FIG. 1-FIG. 7, and all items to find acid reducing foods she needs to eat to suppress her known GERD disease.

After a long day, she decides she wants to charge the invention FIG. 1-FIG. 7, and all items. She can remove the invention FIG. 1-FIG. 7, and all items before bed, place it on the wireless charging station that comes with the product to recharge it and reuse it for the next day.

After she removes the invention FIG. 1-FIG. 7, and all items she places it on the wireless charging station so it can charge. Her spouse grabs it with other gym clothes on accident and throws it in the closet hamper. She can launch the invention item 27 and tap on the screen interface button, “locate”. A digital chip that's embedded in the invention FIG. 1-FIG. 7, and all items will help locate it with a Bluetooth connection. The sound on the wearer's Smartphone will alert more rapidly as she gets closer.

### See FIG. 30-34

Functionality of Screen Interface 7, 1 this is the main screen, 2 Root Hierarchy, 3 List of apps to launch and 4 Apps can be added and removed
She completes the sign up and registration.
She will then simultaneously keeps the invention item 27 running so she can conjunctionally use it with items 1-28, item 30.

### The Construction Details of the Invention as Shown in FIG. 1, FIG. 2, FIG. 3, FIG. 4, FIG. 5

You first impregnate raw material item 1 for the outer layer FIG. 4, item 32 in bulk through a rigorous process.

You start with an industrial size mixing bowl.

You then combined all substrate raw material in with copper oxide powder item 3, in a specific ratio, forming very small particles known as a, masterbatch. This new material referred herein as cloth item 1, has the flexibility to easily “add” a variety of fibers, solid surfaces, and polymers as a simple additive to the normal manufacturing process. Adding master batch to a material is very similar to adding a color and there is no change to existing manufacturing processes nor is there any special equipment required to incorporate the technology into the raw material.

Once you complete the infusion process, you have your base material referred herein as cloth item 1. You can then choose from a number of e-textiles to embed into the cloth item 1, to create health sensors into the cloth item 1 such as elastomeric polymers, conductive materials and flexible substrates. You also have the option of knitting into the raw material conductive or optical structures. Conductive Nano-Composite elastomeric polymers can exhibit changes in electrical conductivity as the material is stretched. These polymers, with variable resistance properties can behave as strain gauges, switches and sensors.

You can then use the elastic material item 6, which is a conductive ribbon that attach to the cloth item 1, which can then be attached to standard electronic connectors to provide a fabric-like, motion-absorbing, wiring hardness useful in dynamic environments including high vibrating industrial or wearable consumer applications.

You also use strips of Peruvian pima Cotton Liner Item 7 at the top and bottom of both inner FIG. 4 item 33 and outer layer FIG. 4 item 32 of the invention.

You then cut the cloth item 1 using heavy machinery into any shape as the outer layer FIG. 4, item 33. You then cut any shape as the inner middle section which provides a broad design using any geometric shape as referred in SUMMARY OF INVENTION, material. See FIG. 4, items 35-43.

You then embed health sensor patch into the end of the outer layer FIG. 4 item 32.

You now have the inner FIG. 4, item 33 and outer layer FIG. 4 item 32 comprising the base of the invention.

Another part of the invention is the use of embedded technology, more specifically the creation of a platform which can be expanded on at any time with newer versions or updates to its firmware and/or hardware and software. The following make up the ecosystem of the invention tying all hardware, software, functions, operations and advancement into a revolutionary new part of the invention. You use a thin case which will be embedded in the back of the invention. It can be made up of any material such as aluminum, plastic or metal, as shown in FIG. 6 item 50.

The next step would be to embed the completely assembled microcontroller circuit board with bios and firmware item, into the back of the invention, which includes the following, but not limited to: Qualcomm Neuromorphic Chip, 2. Memory Chip, 3. Class II Bluetooth Chip, 4. Microphone, sound decoder and codec and amplifier chip, 5. Auto length accelerometer-based board, 6. Altimeter board, 7. 6-Axis accelerometer board, 8. Gyroscope board, 9. Rechargeable pure aluminum-ion battery w/integrated power-saving accelerometer board, 10. Calorie counter board, 11. Pedometer board, GPS Nano-Spider technology and accelerometer boards, pCell chip and board.

You then place the Bluetooth speakers anywhere on the inventions outer layer FIG. 4 item 32. You then code the Smartphone app FIG. 2 item 27 to make the invention whole. You write the Smartphone app FIG. 2 item 27 using Objective-C, HTML, CSS and JavaScript along with other programming languages to make it a completely functional app. You also program it so it's backward compatible, has open API functionality to integrate into existing ecommerce platforms, data bases and other internet connected structures.

You then have the unification of all components in the ecosystem which brings life my invention.

1. I'm claiming that my invention comprising any features described herein, either individually or in combination with any feature, in any configuration,” or a “process to and comprising any process described, in any order, using any modality, uses the following:
A scalable computer board, B. Ai software and firmware, C. Programming language, D. Spider bots, E. pCell wireless chip, F. Health sensors, G. Lateral support belt, H. Back support material, I. Casing Using these components in conjunction with each other they create the invention to offer endless applications such as health monitoring, suggestive technology, health data, education and back support. This provides a unique experience to the user while interconnecting the world of medical, health, fitness and other industries direct with people.
I'm claiming that my invention comprising any features described herein, either individually or in combination with any feature, in any configuration,” or a “process to and comprising any process described, in any order, using any modality, uses the following:
A scalable computer board, B. Ai software, C. Programming language, D. pCell wireless chip, E. Health sensors, F. Lateral support belt, G. Back support material, H. Casing
Using these components in conjunction with each other they create the invention to offer a human connection to smart weapons that react to wearer vitals. This provides a unique experience to the user during war.
I'm claiming that my invention comprising any features described herein, either individually or in combination with any feature, in any configuration,” or a “process to and comprising any process described, in any order, using any modality, uses the following:
